SEQUENCE LISTING (67 sequences)

SEQ ID NO: 1 - protein, 444 aa, Artificial Sequence
  Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 443 aa, Artificial Sequence
  Synthetic Constrict
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - protein, 221 aa, Artificial Sequence
  Synthetic Construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 117 aa, Artificial Sequence
  Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 5 - protein, 116 aa, Artificial Sequence
  Synthetic Construct
QVQLVQSGAEVKKPGASVKVSCKASGYTFTSYWINWVRQAPGQGLEWMGNIWPGSSLTNYNEKFKNRVTMTRDTSTSTVYMELSSLRSEDTAVYYCARLLTGTFAYWGQGTLVTVS

SEQ ID NO: 6 - protein, 113 aa, Artificial Sequence
  Synthetic Construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIK

SEQ ID NO: 7 - protein, 432 aa, Artificial Sequence
  Synthetic Construct
QVQLVESGGGWQPGRSLRLDCKASGITFSNSGMHWVRQAPGKGLEWVAVRWYDGSKRYYADSVKGRFTISRDNSKNTLFLQMNSLRAEDTAVYYCATNDDYWGQGTLVTVSSASTKGPSVFPLAPCSRSTSESTAALGCLVDYFPEPVTVSWNSGALTSGVHTFPAVLQSSGLYSLSSVVTVPSSSLGTTYTCNVDHKPSNTKVDRVESYGPPCPPCPAPEFLGGPSVFLFPPKPKDTLMISRTPEVTCWVDVSQEDPEVQFNWYYDGVEVHNATKPREEQFNSTYRVVSVLTVLHQDWLNGKEYKCKVSNKGLPSSIEKTISKAGQPREPQVYTLPPSQEEMTKNQVSLTCLVKGFYPSDIAVEWESNGQPEKNYKTTPPVLDSDGSFFLYSRLTVDKSRWQEGNVFSCSVMHEALHNHYTQKSLSLSLGK

SEQ ID NO: 8 - protein, 208 aa, Artificial Sequence
  Synthetic Construct
EIVLTQSPATLSLSPGERATLSCRASQSVSSYLAWYQQPGQAPRLLIYDASNRATGIPARFSGSGSGTDFTLTISSLEPEDFAVYYCQQSSNWPRTFGQGTKVEIRTVAAPSVFIFPPSDEQLSGTASVVCLLNNFYPREAVQWKVDNALQSGNSQESVTEQDSDSTYSLSSTLTLSKADYEKHKVYACEVTHQGLSSPVTSFNRGEC

SEQ ID NO: 9 - protein, 447 aa, Artificial Sequence
  Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 218 aa, Artificial Sequence
  Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 432 aa, Artificial Sequence
  Synthetic Construct
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWEPKSCDKTHTCPPCPAPELLGGPSVFLFPPKPKDTLMISRTPEVTCWVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 118 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 13 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 14 - protein, 117 aa, Artificial Sequence
  Synthetic Construct
EVQLLESGGGLVQPGGSLRLSCAASGFTFSSYIMWVRQAPGKGLEWVSSIYPSGGITFYADKGRFTISRDNSKNTLYLQMNSLRAEDTAVYYCARIKLGTVTTVDYWGQGTLVTVSS

SEQ ID NO: 15 - protein, 110 aa, Artificial Sequence
  Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 16 - protein, 439 aa, Artificial Sequence
  Synthetic Construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 17 - protein, 214 aa, Artificial Sequence
  Synthetic Construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 443 aa, Artificial Sequence
  Synthetic Construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 19 - protein, 220 aa, Artificial Sequence
  Synthetic Construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 444 aa, Artificial Sequence
  Synthetic Construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 214 aa, Artificial Sequence
  Synthetic Construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 451 aa, Artificial Sequence
  Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 215 aa, Artificial Sequence
  Synthetic Construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 448 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 128 aa, Artificial Sequence
  Synthetic Construct
QVQLVESGGGLVQPGGSLRLSCAASGFTFSRRCMAWFRQAPGKERERVAKLLTTSGSTYLADSVKGRFTISRDNSKNTVYLQMNSLRAEDTAVYYCAADSFEDPTCTLVTSSGAFQYWGQGTLVTVSS

SEQ ID NO: 27 - protein, 123 aa, Artificial Sequence
  Synthetic Construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 28 - protein, 106 aa, Artificial Sequence
  Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 29 - protein, 447 aa, Artificial Sequence
  Synthetic Construct
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 213 aa, Artificial Sequence
  Synthetic Construct
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 445 aa, Artificial Sequence
  Synthetic Construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 32 - protein, 214 aa, Artificial Sequence
  Synthetic Construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 290 aa, Artificial Sequence
  Synthetic Construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 34 - protein, 5 aa, Artificial Sequence
  Synthetic Construct
SYIMM

SEQ ID NO: 35 - protein, 11 aa, Artificial Sequence
  Synthetic Construct
SIYPSGGITFY

SEQ ID NO: 36 - protein, 11 aa, Artificial Sequence
  Synthetic Construct
IKLGTVTTVDY

SEQ ID NO: 37 - protein, 14 aa, Artificial Sequence
  Synthetic Construct
TGTSSDVGGYNYVS

SEQ ID NO: 38 - protein, 7 aa, Artificial Sequence
  Synthetic Construct
DVSNRPS

SEQ ID NO: 39 - protein, 10 aa, Artificial Sequence
  Synthetic Construct
SSYTSSSTRV

SEQ ID NO: 40 - protein, 450 aa, Artificial Sequence
  Synthetic Construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 216 aa, Artificial Sequence
  Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 42 - protein, 10 aa, Artificial Sequence
  Synthetic Construct
GYTFTSYWIN

SEQ ID NO: 43 - protein, 17 aa, Artificial Sequence
  Synthetic Construct
NIYPGSSLTNYNEKFKN

SEQ ID NO: 44 - protein, 8 aa, Artificial Sequence
  Synthetic Construct
LSTGTFAY

SEQ ID NO: 45 - protein, 17 aa, Artificial Sequence
  Synthetic Construct
KSSQSLWDSGNQKNFLT

SEQ ID NO: 46 - protein, 7 aa, Artificial Sequence
  Synthetic Construct
WTSYRES

SEQ ID NO: 47 - protein, 9 aa, Artificial Sequence
  Synthetic Construct
QNDYFYPHT

SEQ ID NO: 48 - protein, 5 aa, Artificial Sequence
  Synthetic Construct
SYSMN

SEQ ID NO: 49 - protein, 17 aa, Artificial Sequence
  Synthetic Construct
YISSSSSTIDYADSVKG

SEQ ID NO: 50 - protein, 9 aa, Artificial Sequence
  Synthetic Construct
ESGWYLFDY

SEQ ID NO: 51 - protein, 11 aa, Artificial Sequence
  Synthetic Construct
RASQGISSWLA

SEQ ID NO: 52 - protein, 7 aa, Artificial Sequence
  Synthetic Construct
AASSLQS

SEQ ID NO: 53 - protein, 9 aa, Artificial Sequence
  Synthetic Construct
QQYNSYPPT

SEQ ID NO: 54 - protein, 118 aa, Artificial Sequence
  Synthetic Construct
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSS

SEQ ID NO: 55 - protein, 107 aa, Artificial Sequence
  Synthetic Construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIK

SEQ ID NO: 56 - protein, 444 aa, Artificial Sequence
  Synthetic Construct
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 57 - protein, 214 aa, Artificial Sequence
  Synthetic Construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 6 aa, Artificial Sequence
  Synthetic Construct
STYWIS

SEQ ID NO: 59 - protein, 17 aa, Artificial Sequence
  Synthetic Construct
KIYPGDSYTNYSPSFQG

SEQ ID NO: 60 - protein, 8 aa, Artificial Sequence
  Synthetic Construct
RGYGIFDY

SEQ ID NO: 61 - protein, 11 aa, Artificial Sequence
  Synthetic Construct
SGDNIGDQYAH

SEQ ID NO: 62 - protein, 7 aa, Artificial Sequence
  Synthetic Construct
QDKNRPS

SEQ ID NO: 63 - protein, 11 aa, Artificial Sequence
  Synthetic Construct
ATYTGFGSLAV

SEQ ID NO: 64 - protein, 116 aa, Artificial Sequence
  Synthetic Construct
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 65 - protein, 108 aa, Artificial Sequence
  Synthetic Construct
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 66 - protein, 442 aa, Artificial Sequence
  Synthetic Construct
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - protein, 214 aa, Artificial Sequence
  Synthetic Construct
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2022/0241412 A1
(43) Pub. Date: Aug. 4, 2022

(54) COMBINATION THERAPIES USING CDK INHIBITORS

(71) Applicants: Pfizer Inc., New York, NY (US); MERCK PATENT GMBH, Darmstadt (DE)

(72) Inventors: Stephen George Dann, San Diego, CA (US); Cecilia Marianne Oderup, San Francisco, CA (US); Shahram Salek-Ardakani, San Diego, CA (US)

(21) Appl. No.: 17/613,557

(22) PCT Filed: May 20, 2020
(86) PCT No.: PCT/EP2020/064024

§ 371 Date: Nov. 23, 2021

Related U.S. Application Data
(60) Provisional application No. 63/009,433, filed on Apr. 13, 2020.
(60) Provisional application No. 62/852,523, filed on May 24, 2019.

(51) Int. Cl. A61K 39/395; A61K 31/519; A61P 35/00

(52) U.S. Cl. CPC A61K 39/3955; A61P 35/00; A61K 31/519

(57) ABSTRACT

This invention relates to a method for treating cancer by administering a CDK4/6 or a CDK2/4/6 inhibitor in combination with a PD-1 axis binding antagonist, and optionally an OX40 agonist and/or a 4-1BB agonist to a subject in need thereof.

### REFERENCE TO SEQUENCE LISTING

This application is being filed electronically via EFSWeb and includes an electronically submitted sequence listing in .txt format. The .txt file contains a sequence listing entitled “PC72481ApctSEQLISTING_ST25.txt” created on Apr. 15, 2020 and having a size of 22 KB. The sequence listing contained in this .txt file is part of the specification and is herein incorporated by reference in its entirety.

### FIELD OF THE INVENTION

The present invention relates to combination therapies useful for the treatment of cancers. In particular, the invention relates to combination therapies which comprise administering a CDK inhibitor or a pharmaceutically acceptable salt thereof, or a pharmaceutical composition comprising such compounds or salts, in combination with a PD-1 axis binding antagonist, and optionally an OX40 agonist and/or a 4-1BB agonist. The invention also relates to associated methods of treatment, pharmaceutical compositions, and pharmaceutical uses. The methods and compositions are useful for any indication for which the therapeutic is itself useful in the detection, treatment and/or prevention of a disease, disorder, or other condition of a subject.

### BACKGROUND

Cyclin dependent kinases (CDKs) are important cellular enzymes that perform essential functions in regulating eukaryotic cell division and proliferation. The cyclin dependent kinase catalytic units are activated by regulatory subunits known as cyclins. At least sixteen mammalian cyclins have been identified (Johnson DG, Walker CL., Cyclins and Cell Cycle Checkpoints. Annu. Rev. Pharmacol. Toxicol. 1999, 39:295312). Cyclin B/CDK1, cyclin A/CDK2, cyclin E/CDK2, cyclin D/CDK4, cyclin D/CDK6, and likely other heterodynes are important regulators of cell cycle progression. Additional functions of cyclin/CDK heterodynes include regulation of transcription, DNA repair, differentiation and apoptosis (Morgan D O., Cyclin dependent kinases: engines, clocks, and microprocessors. Annu. Rev. Cell. Dev. Biol. 1997, 13:261291).

Cyclin dependent kinase inhibitors have been demonstrated to be useful in treating cancer. Increased activity or temporally abnormal activation of cyclin dependent kinases has been shown to result in the development of human tumors, and human tumor development is commonly associated with alterations in either the CDK proteins themselves or their regulators (Cordon Cardo C., Mutations of cell cycle regulators: biological and clinical implications for human neoplasia. Am. J. Pathol. 1995, 147:545560; Karp JE, Broder S., Molecular foundations of cancer: new targets for intervention. Nat. Med. 1995, 1:309320; Hall M, Peters G. Genetic alterations of cyclins, cyclin dependent kinases, and CDK inhibitors in human cancer. Adv. Cancer Res. 1996, 68:67108). Amplifications of the regulatory subunits of CDKs and cyclins, and mutation, gene deletion, or transcriptional silencing of endogenous CDK inhibitors have also been reported (Smalley et al., Identification of a novel subgroup of melanomas with KIT/cyclin dependent kinase4 overexpression. Cancer Res 2008, 68: 574352).

CDK4/6 inhibitors palbociclib, ribociclib and abemaciclib have been approved for treatment of hormone receptor (HR)-positive, human epidermal growth factor receptor 2 (HER2)-negative advanced or metastatic breast cancer in combination with aromatase inhibitors in post-menopausal women, and in combination with fulvestrant after disease progression following endocrine therapy, (O'Leary et al., Treating cancer with selective CDK4/6 inhibitors. Nature Reviews 2016, 13:417-430). While CDK4/6 inhibitors have shown significant clinical efficacy in HR-positive metastatic breast cancer, as with other kinases their effects may be limited over time by the development of primary or acquired resistance.

Overexpression of CDK2 is associated with abnormal regulation of cell-cycle. The cyclin E/CDK2 complex plays an important role in regulation of the G1/S transition, histone biosynthesis and centrosome duplication. Progressive phosphorylation of Rb by cyclin D/Cdk4/6 and cyclin E/Cdk2 releases the G1 transcription factor, E2F, and promotes S-phase entry. Activation of cyclin A/CDK2 during early S-phase promotes phosphorylation of endogenous substrates that permit DNA replication and inactivation of E2F, for S-phase completion. (Asghar et al., The history and future of targeting cyclin-dependent kinases in cancer therapy, Nat. Rev. Drug. Discov. 2015, 14(2): 130-146).

Cyclin E, the regulatory cyclin for CDK2, is frequently overexpressed in cancer. Cyclin E amplification or overexpression has long been associated with poor outcomes in breast cancer. (Keyomarsi et al., Cyclin E and survival in patients with breast cancer. N Engl J Med. 2002, 347:1566-75). Cyclin E2 (CCNE2) overexpression is associated with endocrine resistance in breast cancer cells and CDK2 inhibition has been reported to restore sensitivity to tamoxifen or CDK4 inhibitors in tamoxifen-resistant and CCNE2 overexpressing cells. (Caldon et al., Cyclin E2 overexpression is associated with endocrine resistance but not insensitivity to CDK2 inhibition in human breast cancer cells. Mol Cancer Ther. 2012, 11:1488-99; Herrera-Abreu et al., Early Adaptation and Acquired Resistance to CDK4/6 Inhibition in Estrogen Receptor-Positive Breast Cancer, Cancer Res. 2016, 76: 2301-2313). Cyclin E amplification also reportedly contributes to trastuzumab resistance in HER2+ breast cancer. (Scaltriti et al., Cyclin E amplification/overexpression is a mechanism of trastuzumab resistance in HER2+ breast cancer patients, Proc Natl Acad Sci. 2011, 108: 3761-6). Cyclin E overexpression has also been reported to play a role in basal-like and triple negative breast cancer (TNBC), as well as inflammatory breast cancer. (Elsawaf & Sinn, Triple Negative Breast Cancer: Clinical and Histological Correlations, Breast Care 2011, 6:273-278; Alexander et al., Cyclin E overexpression as a biomarker for combination treatment strategies in inflammatory breast cancer, Noske, et. al., Detection of CCNE1/URI (19q12) amplification by in situ hybridisation is common in high grade and type II endometrial cancer, Oncotarget 2017, 8: 14897-14911).

Amplification or overexpression of cyclin El (CCNE1) is associated with poor outcomes in ovarian, gastric, endometrial and other cancers. (Nakayama et al., Gene amplification CCNE1 is related to poor survival and potential therapeutic target in ovarian cancer, Cancer 2010, 116: 2621-34; Etemadmoghadam et al., Resistance to CDK2 Inhibitors Is Associated with Selection of Polyploid Cells in CCNE1-Amplified Ovarian Cancer, Clin Cancer Res 2013, 19:5960-71; Au-Yeung et al., Selective Targeting of Cyclin El-Amplified High-Grade Serous Ovarian Cancer by Cyclin-Dependent Kinase 2 and AKT Inhibition, Clin. Cancer Res. 2017, 23:1862-1874; Ayhan et al., CCNE1 copy-number gain and overexpression identify ovarian clear cell carcinoma with a poor prognosis, Modern Pathology 2017, 30:297-303; Ooi et al., Gene amplification of CCNE1, CCND1, and CDK6 in gastric cancers detected by multiplex ligation-dependent probe amplification and fluorescence in situ hybridization, Hum Pathol. 2017, 61:58-67; Noske et al., Detection of CCNE1/URI (19q12) amplification by in situ hybridization is common in high grade and type II endometrial cancer, Oncotarget 2017, 8:14794-14805).

Palbociclib, or 6-acetyl-8-cyclopentyl-5-methyl-2-(5-piperazin-1-yl-pyridin-2-ylamino)-8H-pyrido[2,3-d]pyrimidin-7-one (also referred to as “palbo,” “Palbo” or “PD-0332991”) is a potent and selective inhibitor of CDK4 and CDK6, having the structure:

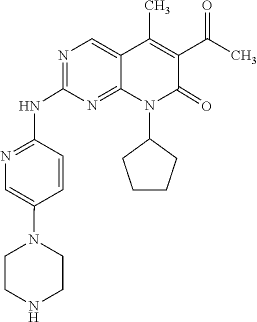 [SMILES: CC(=O)c1c(C)c2cnc(Nc3ccc(N4CCNCC4)cn3)nc2n(C2CCCC2)c1=O]

Palbociclib is described in WHO Drug Information, 2013, Vol. 27, No. 2, page 172. Palbociclib and pharmaceutically acceptable salts thereof, are disclosed in International

Publication No. WO 2003/062236 and U.S. Pat. Nos. 6,936,612, 7,208,489 and 7,456,168; International Publication No. WO 2005/005426 and U.S. Pat. Nos. 7,345,171 and 7,863,278; International Publication No. WO 2008/032157 and U.S. Pat. No. 7,781,583; and International Publication No. WO 2014/128588. The contents of each of the foregoing references are incorporated herein by reference in their entirety.

The compound PF-06873600, or 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, is a potent and selective inhibitor of CDK2, CDK4 and CDK6, having the structure:

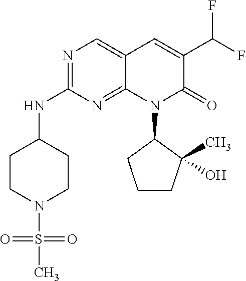 [SMILES: C[C@@]1(O)CCC[C@H]1n1c(=O)c(C(F)F)cc2cnc(NC3CCN(S(C)(=O)=O)CC3)nc21]

PF-06873600 and pharmaceutically acceptable salts thereof, are disclosed in International Publication No. WO 2018/033815 published Feb. 22, 2018. The contents of that reference are incorporated herein by reference in its entirety. The programmed death 1 (PD-1) receptor and PD-1 ligands 1 and 2 (PD-L1 and PD-L2, respectively) play integral roles in immune regulation. PD-1 is expressed by activated T cells, B cells, and myeloid cells. Further, the majority of tumor infiltrating T lymphocytes overexpress PD-1 relative to T lymphocytes in normal tissues and peripheral blood T lymphocytes (Ahmadzadeh et al., Tumor antigen-specific CD8 T cells infiltrating the tumor express high levels of PD-1 and are functionally impaired, Blood, 2009 114(8):1537). PD-1 has two known ligands, programmed death ligand 1 (PD-L1) and programmed death ligand 2 (PD-L2). PD-1 is activated by PD-L1 (also referred to as B7-H1, B7-4, CD274, and B7-H) and PD-L2 expressed by stromal cells, tumor cells, or both, initiating T-cell death and localized immune suppression (Dong et al., B7-H1, a third member of the B7 family, co-stimulates T-cell proliferation and interleukin-10 secretion, Nat Med 1999; 5:1365-69; Freeman et al., Engagement of the PD-1 immunoinhibitory receptor by a novel B7 family member leads to negative regulation of lymphocyte activation, J Exp Med 2000; 192:1027-34), potentially providing an immune-tolerant environment for tumor development and growth. Conversely, inhibition of this interaction can enhance local T-cell responses and mediate antitumor activity in nonclinical animal models (Iwai Y, et al., Involvement of PD-L1 on tumor cells in the escape from host immune system and tumor immunotherapy by PD-L1 blockade, Proc Natl Acad Sci USA 2002; 99:12293-97).

PD-L1 is a cell-surface protein and member of the B7 family. PD-L1 is found on almost all types of lymphohematopoietic cells and is expressed at low levels by resting T cells, B cells, macrophages and dendritic cells and is further up regulated by an anti-CD40 antibody for B cells, anti-CD3 antibody for T cells, anti-CD40 antibody, IFNγ and granulocyte macrophage colony-stimulating factor (GM-CSF) for macrophages and/or anti-CD40 antibody, IFNγ, IL-4, IL-12 and GM-CSF for Dendritic cells (DCs). PD-L1 is also expressed by some non-hemoatopoietic cells and is overexpressed in many cancers, wherein its overexpression is often associated with poor prognosis (Okazaki T et al., PD-1 and PD-1 ligands: from discovery to clinical application, Intern. Immun. 2007 19(7):813) (Thompson R H et al., Tumor B7-H1 is associated with poor prognosis in renal cell carcinoma patients with long-term follow-up, Cancer Res 2006, 66(7):3381). Interestingly, the majority of tumor infiltrating T lymphocytes predominantly express PD-1, in contrast to T lymphocytes in normal tissues and peripheral blood. PD-1 on tumor-reactive T cells can contribute to impaired antitumor immune responses (Ahmadzadeh et al., Tumor antigen-specific CD8 T cells infiltrating the tumor express high levels of PD-1 and are functionally impaired, Blood 2009 1 14(8): 1537). This may be due to exploitation of PD-L1 signaling mediated by PD-L1 expressing tumor cells interacting with PD-1 expressing T cells to result in attenuation of T cell activation and evasion of immune surveillance (Sharpe et al., The B7-CD28 superfamily, Nat Rev 2002) (Keir ME et al., PD-1 and its ligands in tolerance and immunity Annu. Rev. Immunol. 2008, 26:677). Therefore, inhibition of the PD-L1 /PD-1 interaction may enhance CD8+T cell-mediated killing of tumors.

The other known ligand for PD-1, PD-L2, also known as B7-DC, Btdc, and CD273, is a cell surface protein. PD-L2 is expressed by antigen presenting cells, including dendritic cells, with expression also found in other non-hematopoietic tissues.

The inhibition of PD-1 axis signaling through its direct ligands (e.g., PD-L1, PD-L2) has been proposed as a means to enhance T cell immunity for the treatment of cancer (e.g., tumor immunity). Moreover, similar enhancements to T cell immunity have been observed by inhibiting the binding of PD-L1 to the binding partner B7-1 (Ribas A. and Wolchok J., Cancer immunotherapy using checkpoint blockade, Science, 2018, 359: 1350-1355).

The OX40 receptor (also known as CD134, TNFRSF4, ACT-4, ACT35, and TXGP1L) is a member of the TNF receptor superfamily. OX40 is found to be expressed on activated CD4+ and CD8+ T-cells. High numbers of OX40+ T cells have been demonstrated within tumors (tumor infiltrating lymphocytes) and in the draining lymph nodes of cancer patients (Weinberg, A. et al., Assessment of activity of an adhesion molecule CD134 and CD137 in colorectal cancer patients, J. Immunol. 2000, 164:2160-69; Petty, J. et al., Survival in human colorectal cancer correlates with expression of the T-cell costimulatory molecule OX-40 (CD134), Am. J. Surg. 2002,183: 512-518). It was shown in tumor models in mice that engagement of OX40 in vivo during tumor priming significantly delayed and prevented the appearance of tumors as compared to control treated mice (Weinberg et al., 2000). Therefore, it has been contemplated to enhance the immune response of a mammal to an antigen by engaging OX40 through the use of an OX40 binding agent (WO 1999/042585; Weinberg et al., 2000).

4-1BB (also known as CD137 and TNFRSF9), which was first identified as an inducible costimulatory receptor expressed on activated T cells, is a membrane spanning glycoprotein of the Tumor Necrosis Factor (TNF) receptor superfamily. Current understanding of 4-1BB indicates that expression is generally activation dependent and encompasses a broad subset of immune cells including activated NK and NKT cells; regulatory T cells; dendritic cells (DC) including follicular DC; stimulated mast cells, differentiating myeloid cells, monocytes, neutrophils, eosinophils, and activated B cells. 4-1BB expression has also been demonstrated on tumor vasculature (19-20) and atherosclerotic endothelium. The ligand that stimulates 4-1BB (4-1BBL) is expressed on activated antigen presenting cells (APCs), myeloid progenitor cells and hematopoietic stem cells. 4-1BB agonist mAbs increase costimulatory molecule expression and markedly enhance cytolytic T lymphocyte responses, resulting in anti-tumor efficacy in various models. 4-1BB agonist mAbs have demonstrated efficacy in prophylactic and therapeutic settings and both monotherapy and combination therapy tumor models and have established durable anti-tumor protective T cell memory responses

Improved therapies for treating, stabilizing, preventing, and/or delaying development of various cancers, including cancers resistant to CDK inhibitors, comprise a large unmet medical need and the identification of novel combination regimens are required to improve treatment outcome. Preferred combination therapies of the present invention show greater efficacy than treatment with the individual therapeutic agents alone.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY OF THE INVENTION

This invention relates to therapeutic methods, combinations, and pharmaceutical compositions for use in the treatment of cancer. Also provided are combination therapies comprising the compounds of the invention, in combination with other therapeutic agents. The present invention also provides kits comprising one or more of the compositions of the invention.

In one aspect, the invention provides a method for treating cancer comprising administering to a subject in need thereof an amount of a cyclin dependent kinase (CDK) inhibitor in combination with an amount of a PD-1 axis binding antagonist, wherein the amounts together are effective in treating cancer, and wherein the CDK inhibitor is an inhibitor of CDK4 and CDK6 (CDK4/6 inhibitor), or an inhibitor of CDK2, CDK4 and CDK6 (CDK2/4/6 inhibitor).

In some embodiments, the method further comprises the combined administration to the subject of an amount of: a. an OX40 agonist; b. a 4-1BB agonist; or c. an OX40 agonist and a 4-1BB agonist; wherein the amounts together are effective in treating cancer.

In some such embodiments, the PD-1 axis binding antagonist in any of the above methods comprises a PD-1 binding antagonist, a PD-L1 binding antagonist, or a PD-L2 binding antagonist.

In a specific embodiment, the PD-1 axis binding antagonist comprises a PD-1 binding antagonist. In some such embodiments, the PD-1 binding antagonist inhibits the binding of PD-1 to its ligand binding partners. In some embodiments, the PD-1 binding antagonist inhibits the binding of PD-1 to PD-LI. In some embodiments, the PD-1 binding antagonist inhibits the binding of PD-1 to PD-L2. In some embodiments, the PD-1 binding antagonist inhibits the binding of PD-1 to both PD-L1 and PD-L2.

In a specific embodiment, the PD-1 binding antagonist is AMP-224. In one embodiment, the PD-1 binding antagonist is an anti-PD-1 antibody. In some such embodiments, the anti-PD-1 antibody is nivolumab (MDX 1106), pembrolizumab (MK-3475), pidilizumab (CT-011), cemiplimab (REGN2810), tislelizumab (BGB-A317), spartalizumab (PDR001), RN888, mAb15, MEDI-0680 (AMP-514), BGB-108, or AGEN-2034, or a combination thereof.

In some embodiments of the treatment methods as described herein, the PD-1 axis binding antagonist comprises a PD-L1 binding antagonist. In certain embodiments, wherein the PD-L1 binding antagonist inhibits the binding of PD-L1 to PD-1. In some embodiments, the PD-L1 binding antagonist inhibits the binding of PD-L1 to B7-1. In some embodiments, the PD-L1 binding antagonist inhibits the binding of PD-L1 to both PD-1 and B7-1.

In a particular embodiment, the PD-L1 binding antagonist is an anti-PD-L1 antibody. In a more specific embodiment, the anti-PD-L1 antibody is BMS-936559 (MDX-1105), AMP-714, atezolizumab (MPDL3280A), durvalumab (MEDI4736), avelumab, or an antibody comprising a VH region produced by the expression vector with ATCC Accession No. PTA-121183 and having the VL region produced by the expression vector with ATCC Accession No. PTA-121182, or a combination thereof.

In one aspect, the invention provides a method for treating cancer comprising administering to a subject in need thereof, an amount of a cyclin dependent kinase (CDK) inhibitor in combination with an amount of a PD-1 axis binding antagonist and an amount of an OX40 agonist, wherein the amounts together are effective in treating cancer, and wherein the CDK inhibitor is an inhibitor of CDK4 and CDK6 (CDK4/6 inhibitor), or an inhibitor of CDK2, CDK4 and CDK6 (CDK2/4/6 inhibitor).

In some embodiments of the treatment methods as described herein, the OX40 agonist is an anti-OX40 antibody, an OX40L agonist fragment, an OX40 oligomeric receptor, a trimeric OX40L-Fc protein or an OX40 immunoadhesin, or a combination thereof. In a specific embodiment, the OX40 agonist is an anti-OX40 antibody. In some such embodiments, the anti-OX40 antibody is MEDI6469, MEDI0562, MEDI6383, MOXR0916, or GSK3174998, or a combination thereof. In a particular embodiment, the anti-OX40 antibody is a full-length human IgG-1 antibody. In some embodiments, the OX40 agonist is an OX40L agonist fragment comprising one or more extracellular domains of OX40L.

In one aspect, the invention provides a method for treating cancer comprising administering to a subject in need thereof, an amount of a cyclin dependent kinase (CDK) inhibitor in combination with an amount of a PD-1 axis binding antagonist and an amount of a 4-1 BB agonist, wherein the amounts together are effective in treating cancer, and wherein the CDK inhibitor is an inhibitor of CDK4 and CDK6 (CDK4/6 inhibitor), or an inhibitor of CDK2, CDK4 and CDK6 (CDK2/4/6 inhibitor).

In another aspect, the invention provides a method for treating cancer comprising administering to a subject in need thereof, an amount of a cyclin dependent kinase (CDK) inhibitor in combination with an amount of a PD-1 axis binding antagonist, an amount of an OX40 agonist and an amount of a 4-1 BB agonist, wherein the amounts together are effective in treating cancer, and wherein the CDK inhibitor is an inhibitor of CDK4 and CDK6 (CDK4/6 inhibitor), or an inhibitor of CDK2, CDK4 and CDK6 (CDK2/4/6 inhibitor).

In some embodiments, the 4-1BB agonist is an anti-4-1BB antibody. In a specific embodiment, the 4-1BB agonist is utomilumab (PF-05082566), 1D8, 3EIor, 4B4, H4-1BB-M127, BBK2, 145501, antibody produced by cell line deposited as ATCC No. HB-11248, 5F4, C65-485, urelumab (BMS-663513), 20H4.9-IgG-1 (BMS-663031), 4E9, BMS-554271, BMS-469492, 3H3, BMS-469497, 3EI, 53A2, or 3B8.

In some embodiments of the treatment methods as described herein, the CDK inhibitor is a CDK4/6 inhibitor. In some such embodiments, the CDK4/6 inhibitor is palbociclib, or a pharmaceutically acceptable salt thereof.

In some embodiments of the treatment methods as described herein, the CDK inhibitor is a CDK2/4/6 inhibitor. In some such embodiments, the CDK2/4/6 inhibitor is 6-(difluoromethyl)-8-((1R,2R)-2-hydroxy-2-methylcyclopentyl)-2-(1-(methylsulfonyl)-piperidin-4-ylamino)pyrido[2,3-d]pyrimidin-7(8H)-one, or a pharmaceutically acceptable salt thereof.

In some embodiments of the methods as described herein, the subject is a human. In some embodiments of the methods as described herein, the cancer is a solid tumor.

In some embodiments of the methods as described herein, the cancer is a hematologic cancer.

In some embodiments of the treatment methods as described herein, the cancer is selected from the group consisting of brain cancer, head/neck cancer (including squamous cell carcinoma of the head and neck (SCCHN)), prostate cancer, ovarian cancer, bladder cancer (including urothelial carcinoma, also known as transitional cell carcinoma (TCC)), lung cancer (including squamous cell carcinoma, small cell lung cancer (SCLC), and non-small cell lung cancer (NSCLC)), breast cancer, bone cancer, colorectal cancer, kidney cancer, liver cancer (including hepatocellular carcinoma (HCC)), stomach cancer, pancreatic cancer, esophageal cancer , cervical cancer, sarcoma, skin cancer (including melanoma and Merkel cell carcinoma (MCC)), multiple myeloma, mesothelioma, malignant rhabdoid tumors, neuroblastoma, diffuse intrinsic pontine glioma (DIPG), carcinoma, lymphoma, diffuse large B-cell lymphoma (DLBCL), primary mediastinal B-cell lymphoma (PMBCL), follicular lymphoma, acute lymphoblastic leukemia (ALL), acute myeloid leukemia (AML), chronic lymphocytic leukemia (CLL), chronic myeloid leukemia (CML), follicular lymphoma, Hodgkin's lymphoma (HL), classical Hodgkin lymphoma (cHL), mantle cell lymphoma (MCL), multiple myeloma (MM), myeloid cell leukemia-1 protein (Mcl-1), myelodysplastic syndrome (MDS), non-Hodgkin's lymphoma (NHL), small lymphocytic lymphoma (SLL), and SWI/SNF-mutant cancer.

In certain embodiments, the methods of the present invention further comprise administering chemotherapy, radiotherapy, immunotherapy, or phototherapy, or any combinations thereof to the subject.

In one aspect, the invention provides a combination comprising a. (i) palbociclib, or a pharmaceutically acceptable salt thereof; and (ii) a PD-1 binding antagonist; b. (i) palbociclib, or a pharmaceutically acceptable salt thereof; (ii) a PD-1 binding antagonist; and (iii) an OX40 agonist; c. (i) palbociclib, or a pharmaceutically acceptable salt thereof; (ii) a PD-1 binding antagonist; and (iii) a 4-1BB agonist; or d. (i) palbociclib, or a pharmaceutically acceptable salt thereof; (ii) a PD-1 binding antagonist; (iii) an OX40 agonist; and (iv) a 4-1BB agonist; for use in the treatment of cancer in a subject.

In one aspect, the invention provides a combination comprising a. (i) palbociclib, or a pharmaceutically acceptable salt thereof; and (ii) a PD-L1 binding antagonist; b. (i) palbociclib, or a pharmaceutically acceptable salt thereof; (ii) a PD-L1 binding antagonist; and (iii) an OX40 agonist; c. (i) palbociclib, or a pharmaceutically acceptable salt thereof; (ii) a PD-L1 binding antagonist; and (iii) a 4-1BB agonist; d. (i) palbociclib, or a pharmaceutically acceptable salt thereof; (ii) a PD-L1 binding antagonist; (iii) an OX40 agonist; and (iv) a 4-1BB agonist; or e. (i) palbociclib, or a pharmaceutically acceptable salt thereof; (ii) a PD-1 binding antagonist; (iii) a PD-L1 binding antagonist; (iv) an OX40 agonist; and (v) a 4-1BB agonist; for use in the treatment of cancer in a subject.

In one aspect, the invention provides a combination comprising a. (i) 6-(difluoromethyl)-8-((1R,2R)-2-hydroxy-2-methylcyclopentyl)-2-(1-(methylsulfonyl)-piperidin-4-ylamino)pyrido[2,3-d]pyrimidin-7(8H)-one, or a pharmaceutically acceptable salt thereof; and (ii) a PD-1 binding antagonist; b. (i) 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof; (ii) a PD-1 binding antagonist; and (iii) an OX40 agonist; c. (i) 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof; (ii) a PD-1 binding antagonist; and (iii) a 4-1BB agonist; or d. (i) 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof; (ii) a PD-1 binding antagonist; (iii) an OX40 agonist; and (iv) a 4-1BB agonist; for use in the treatment of cancer in a subject.

In one aspect, the invention provides a combination comprising a. (i) 6-(difluoromethyl)-8-((1R,2R)-2-hydroxy-2-methylcyclopentyl)-2-(1-(methylsulfonyl)-piperidin-4-ylamino)pyrido[2,3-d]pyrimidin-7(8H)-one, or a pharmaceutically acceptable salt thereof; and (ii) a PD-L1 binding antagonist; b. (i) 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof; (ii) a PD-L1 binding antagonist; and (iii) an OX40 agonist; c. (i) 6-(difluoromethyl)-8-((1R,2R)-2-hydroxy-2-methylcyclopentyl)-2-(1-(methylsulfonyl)piperidin-4-ylamino)pyrido[2,3- d]pyrimidin-7(8H)-one, or a pharmaceutically acceptable salt thereof; (ii) a PD-L1 binding antagonist; and (iii) a 4-1 BB agonist; d. (i) 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof; (ii) a PD-L1 binding antagonist; (iii) an OX40 agonist; and (iv) a 4-1 BB agonist; or e. (i) 6-(difluoromethyl)-8-((1R,2R)-2-hydroxy-2-methylcyclopentyl)-2-(1-(methylsulfonyl)piperidin-4- ylamino)pyrido[2,3-d]pyrimidin-7(8H)-one, or a pharmaceutically acceptable salt thereof; (ii) a PD-1 binding antagonist, (iii) a PD-L1 binding antagonist, (iv) an OX40 agonist, and (v) an anti-4-1BB antibody; for use in the treatment of cancer in a subject.

In some embodiments of the combinations herein, the PD-1 binding antagonist is an anti-PD-1 antibody; the PD-L1 binding antagonist is an anti-PD-L1 antibody; the OX40 agonist is an anti-OX40 antibody; and/or the 4-1BB agonist is an anti-4-1BB antibody.

In specific embodiments of the combinations herein, the combination is synergistic. In some embodiments of the combinations herein, the subject is a human. In some embodiments of the combinations herein, the cancer is a solid tumor. In some embodiments of the combinations herein, the cancer is a hematologic cancer. In some embodiments of the combinations as described herein, the cancer is selected from the group consisting of brain cancer, head/neck cancer (including squamous cell carcinoma of the head and neck (SCCHN)), prostate cancer, ovarian cancer, bladder cancer (including urothelial carcinoma, also known as transitional cell carcinoma (TCC)), lung cancer (including squamous cell carcinoma, small cell lung cancer (SCLC), and non-small cell lung cancer (NSCLC)), breast cancer, bone cancer, colorectal cancer, kidney cancer, liver cancer (including hepatocellular carcinoma (HCC)), stomach cancer, pancreatic cancer, esophageal cancer , cervical cancer, sarcoma, skin cancer (including melanoma and Merkel cell carcinoma (MCC)), multiple myeloma, mesothelioma, malignant rhabdoid tumors, neuroblastoma, diffuse intrinsic pontine glioma (DIPG), carcinoma, lymphoma, diffuse large B-cell lymphoma (DLBCL), primary mediastinal B-cell lymphoma (PMBCL), follicular lymphoma, acute lymphoblastic leukemia (ALL), acute myeloid leukemia (AML), chronic lymphocytic leukemia (CLL), chronic myeloid leukemia (CML), follicular lymphoma, Hodgkin's lymphoma (HL), classical Hodgkin lymphoma (cHL), mantle cell lymphoma (MCL), multiple myeloma (MM), myeloid cell leukemia-1 protein (Mcl-1), myelodysplastic syndrome (MDS), non-Hodgkin's lymphoma (NHL), small lymphocytic lymphoma (SLL), and SWI/SNF-mutant cancer.

In some embodiments, the cancer is breast cancer. Breast cancer may include luminal A, luminal B, triple negative/basal-like, or HER2-enriched subtypes. Breast cancers may be estrogen receptor (ER)-positive and/or progesterone receptor (PR)-positive, alternatively referred to as hormone receptor (HR)-positive. HR-positive breast cancers may be human epidermal growth factor receptor 2 (HER2)-negative (i.e., HR+/HER2−) or HER2-positive (i.e., HR+/HER2+). HR-negative breast cancers may be HER2-positive (i.e., HR−/HER2+) or HER-negative (HR−/HER2−), i.e. “triple negative” breast cancer (TNBC). In some embodiments, the breast cancer demonstrates primary or acquired resistance to endocrine therapy, anti-HER2 agents and/or CDK4/CDK6 inhibitors. In some embodiments, the breast cancer is advanced or metastatic breast cancer. In some embodiments of the foregoing, the breast cancer is characterized by amplification or overexpression of CCNE1 and/or CCNE2.

In one aspect, the invention provides a kit comprising: a. (i) a pharmaceutical composition comprising a CDK inhibitor and a pharmaceutically acceptable carrier; (ii) a pharmaceutical composition comprising a PD-1 binding antagonist and a pharmaceutically acceptable carrier; b. (i) a pharmaceutical composition comprising a CDK inhibitor and a pharmaceutically acceptable carrier; (ii) a pharmaceutical composition comprising a PD-1 binding antagonist and a pharmaceutically acceptable carrier; (iii) a pharmaceutical composition comprising an OX40 agonist and a pharmaceutically acceptable carrier; c. (i) a pharmaceutical composition comprising a CDK inhibitor and a pharmaceutically acceptable carrier; (ii) a pharmaceutical composition comprising a PD-1 binding antagonist and a pharmaceutically acceptable carrier; (iii) a pharmaceutical composition comprising a 4-1BB agonist and a pharmaceutically acceptable carrier; or d. (i) a pharmaceutical composition comprising a CDK inhibitor and a pharmaceutically acceptable carrier; (ii) a pharmaceutical composition comprising PD-1 binding antagonist and a pharmaceutically acceptable carrier; (iii) a pharmaceutical composition comprising an OX40 agonist and a pharmaceutically acceptable carrier; (iv) a pharmaceutical composition comprising a 4-1BB agonist and a pharmaceutically acceptable carrier; and instructions for dosing of the pharmaceutical compositions for the treatment of cancer.

In some embodiments of the above kits, the PD-1 binding antagonist is an anti-PD-1 antibody; the OX40 agonist is an anti-OX40 antibody; and/or the 4-1BB agonist is an anti-4-1BB antibody.

In one aspect, the invention provides a kit comprising: a. (i) a pharmaceutical composition comprising a CDK inhibitor and a pharmaceutically acceptable carrier; (ii) a pharmaceutical composition comprising a PD-L1 binding antagonist and a pharmaceutically acceptable carrier; b. (i) a pharmaceutical composition comprising a

CDK inhibitor and a pharmaceutically acceptable carrier; (ii) a pharmaceutical composition comprising a PD-L1 binding antagonist and a pharmaceutically acceptable carrier; (iii) a pharmaceutical composition comprising an OX40 agonist and a pharmaceutically acceptable carrier; c. (i) a pharmaceutical composition comprising a CDK inhibitor and a pharmaceutically acceptable carrier; (ii) a pharmaceutical composition comprising a PD-L1 binding antagonist and a pharmaceutically acceptable carrier; (iii) a pharmaceutical composition comprising a 4-1BB agonist and a pharmaceutically acceptable carrier; d. (i) a pharmaceutical composition comprising a CDK inhibitor and a pharmaceutically acceptable carrier; (ii) a pharmaceutical composition comprising PD-L1 binding antagonist and a pharmaceutically acceptable carrier; (iii) a pharmaceutical composition comprising an OX40 agonist and a pharmaceutically acceptable carrier; (iv) a pharmaceutical composition comprising a 4-1BB agonist and a pharmaceutically acceptable carrier; or e. (i) a pharmaceutical composition comprising a CDK inhibitor and a pharmaceutically acceptable carrier; (ii) a pharmaceutical composition comprising a PD-1 binding antagonist and a pharmaceutically acceptable carrier; (iii) a pharmaceutical composition comprising PD-L1 binding antagonist and a pharmaceutically acceptable carrier; (iv) a pharmaceutical composition comprising an OX40 agonist and a pharmaceutically acceptable carrier; (v) a pharmaceutical composition comprising a 4-1BB agonist and a pharmaceutically acceptable carrier; and instructions for dosing of the pharmaceutical compositions for the treatment of cancer.

In some embodiments of the kits as described herein, the PD-L1 binding antagonist is an anti-PD-L1 antibody; the OX40 agonist is an anti-OX40 antibody; and/or the 4-1BB agonist is an anti-4-1BB antibody.

In some embodiments of the kits as described herein, the CDK inhibitor is a CDK4/6 inhibitor. In specific embodiments of the kits as described herein, the CDK4/6 inhibitor is palbociclib, or a pharmaceutically acceptable salt thereof.

In some embodiments of the kits as described herein, the CDK inhibitor is a CDK2/4/6 inhibitor. In specific embodiments of the kits as described herein, the CDK inhibitor is 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof.

### BRIEF DESCRIPTION OF THE DRAWINGS

FIG. 1 depicts syngeneic MC38 tumor growth inhibition comparing Isotype/Vehicle control with immune checkpoint blockade (PD-L1 (PF-06834635), OX40 (PF-07201252) or 4-1BB (PF-072188CDK4/6 inhibition (palbociclib) and the combination of checkpoint blockade with CDK4/6 inhibition (anti-PD-L1 antibody (PF-06834635)/anti-OX40 antibody (PF-07201252)/anti-4-1BB antibody (PF-07218859)+palbociclib) as cohort mean tumor volume (error bars represent standard error of the mean).

FIG. 2A depicts syngeneic MC38 tumor growth inhibition response to isotype and vehicle control from FIG. 1 as individual tumor growth curves.

FIG. 2B depicts syngeneic MC38 tumor growth inhibition response to immune checkpoint blockade (anti-PD-L1 antibody (PF-06834635)/anti-OX40 antibody (PF-07201252)/anti-4-1BB antibody (PF-07218859)) from FIG. 1 as individual tumor growth curves.

FIG. 2C depicts syngeneic MC38 tumor growth inhibition response to CDK4/6 inhibition (palbociclib) from FIG. 1 as individual tumor growth curves. FIG. 2D depicts syngeneic MC38 tumor growth inhibition response to the combination of checkpoint blockade with CDK4/6 inhibition (anti-PD-L1 antibody (PF-06834635)/anti-OX40 antibody (PF-07201252)/anti-4-1BB antibody (PF-07218859) +palbociclib) from FIG. 1 as individual tumor growth curves.

FIG. 3 depicts syngeneic MC38 tumor growth inhibition comparing Isotype/Vehicle control with immune checkpoint blockade (anti-PD-L1 antibody (PF-06834635)/anti-OX40 antibody (PF-07201252)/anti-4-1BB antibody (PF-07218859), PD-L1 (PF-06834635)/anti-OX40 antibody (PF-07201252), PD-L1 (PF-06834635)/anti-4-1BB antibody (PF-07218859), anti-PD-L1 antibody (PF-06834635)), CDK2/4/6 inhibition (PF-06873600) and the combination of checkpoint blockade with CDK2/4/6 inhibition ((anti-PD-L1 antibody (PF-06834635)/anti-OX40 antibody (PF-07201252)/anti-4-1BB antibody (PF-07218859)+CDK2/4/6 inhibitor (PF-06873600), anti-PD-L1 antibody (PF-06834635)/anti-OX40 antibody (PF-07201252) +CDK2/4/6 inhibitor (PF-06873600), anti-PD-L1 antibody (PF-06834635)/anti-4-1BB antibody (PF-07218859)+CDK2/4/6 inhibitor (PF-06873600), anti-PD-L1 antibody (PF-06834635)+CDK2/4/6 inhibitor (PF-06873600)) as cohort mean tumor volume (error bars represent standard error of the mean).

FIG. 4A depicts syngeneic MC38 tumor growth inhibition response to isotype and vehicle control from FIG. 3 as individual tumor growth curves.

FIG. 4B depicts syngeneic MC38 tumor growth inhibition response to CDK2/4/6 inhibition (PF-06873600) from FIG. 3 as individual tumor growth curves.

FIG. 4C depicts syngeneic MC38 tumor growth inhibition response to immune checkpoint blockade (anti-PD-L1 antibody (PF-06834635)/anti-OX40 antibody (PF-07201252)) from FIG. 3 as individual tumor growth curves.

FIG. 4D depicts syngeneic MC38 tumor growth inhibition response to the combination of checkpoint blockade with CDK2/4/6 inhibition (anti-PD-L1 antibody (PF-06834635)/anti-OX40 antibody (PF-07201252) +CDK2/4/6 inhibitor (PF-06873600)) from FIG. 3 as individual tumor growth curves.

FIG. 4E depicts syngeneic MC38 tumor growth inhibition response to immune checkpoint blockade (anti-PD-L1 antibody (PF-06834635)/anti-4-1BB antibody (PF-07218859)) from FIG. 3 as individual tumor growth curves.

FIG. 4F depicts syngeneic MC38 tumor growth inhibition response to the combination of checkpoint blockade with CDK2/4/6 inhibition (anti-PD-L1 antibody (PF-06834635)/anti-4-1BB antibody (PF-07218859) +CDK2/4/6 inhibitor (PF-06873600)) from FIG. 3 as individual tumor growth curves.

FIG. 4G depicts syngeneic MC38 tumor growth inhibition response to immune checkpoint blockade (anti-PD-L1 antibody (PF-06834635)) from FIG. 3 as individual tumor growth curves.

FIG. 4H depicts syngeneic MC38 tumor growth inhibition response to the combination of checkpoint blockade with CDK2/4/6 inhibition (anti-PD-L1 antibody (PF-06834635)+CDK2/4/6 inhibitor (PF-06873600)) from FIG. 3 as individual tumor growth curves.

FIG. 4I depicts syngeneic MC38 tumor growth inhibition response to immune checkpoint blockade (anti-PD-L1 antibody (PF-06834635)/anti-OX40 antibody (PF-07201252)/anti-4-1BB antibody (PF-07218859)) from FIG. 3 as individual tumor growth curves.

FIG. 4J depicts syngeneic MC38 tumor growth inhibition response to the combination of checkpoint blockade with CDK2/4/6 inhibition (anti-PD-L1 antibody (PF-06834635)/anti-OX40 antibody (PF-07201252)/anti-4-1BB antibody (PF-07218859)+CDK2/4/6 inhibitor (PF-06873600)) from FIG. 3 as individual tumor growth curves.

FIG. 5 depicts syngeneic 4T1 tumor growth inhibition comparing Isotype/Vehicle control with immune checkpoint blockade (anti-PD-L1 antibody (PF-06834635)/anti-OX40 antibody (PF-07201252)/anti-4-1BB antibody (PF-07218859)), CDK2/4/6 inhibition (PF-06873600) and the combination of checkpoint blockade with CDK2/4/6 inhibition (anti-PD-L1 antibody (PF-06834635)/anti-OX40 antibody (PF-07201252)/anti-4-1BB antibody (PF-07218859), (anti-PD-L1 antibody (PF-06834635)/anti-OX40 antibody (PF-07201252)/anti-4-1BB antibody (PF-07218859)+CDK2/4/6 inhibitor (PF-06873600)) as cohort mean tumor volume (error bars represent standard error of the mean).

FIG. 6A depicts syngeneic 4T1 tumor growth inhibition response to isotype and vehicle control from FIG. 5 as individual tumor growth curves.

FIG. 6B depicts syngeneic 4T1 tumor growth inhibition response to immune checkpoint blockade (anti-PD-L1 antibody (PF-06834635)/anti-OX40 antibody (PF-07201252)/anti-4-1BB antibody (PF-07218859)) from FIG. 5 as individual tumor growth curves.

FIG. 6C depicts syngeneic 4T1 tumor growth inhibition response to CDK2/4/6 inhibition (PF-06873600) from FIG. 5 as individual tumor growth curves.

FIG. 6D depicts syngeneic 4T1 tumor growth inhibition response to the combination of checkpoint blockade with CDK2/4/6 inhibition (anti-PD-L1 antibody (PF-06834635)/anti-OX40 antibody (PF-07201252)/anti-4-1BB antibody (PF-07218859)+CDK2/4/6 inhibitor (PF-06873600)) from FIG. 5 as individual tumor growth curves.

### DETAILED DESCRIPTION

Each of the embodiments described below can be combined with any other embodiment described herein not inconsistent with the embodiment with which it is combined. Furthermore, each of the embodiments described herein envisions within its scope pharmaceutically acceptable salts of the small molecule compounds described herein. Accordingly, the phrase “or a pharmaceutically acceptable salt thereof” is implicit in the description of all small molecule compounds described herein.

### I. Abbreviations

Throughout the detailed description and examples of the invention the following abbreviations will be used:
BID One dose twice daily
CDR Complementarity determining region
CHO Chinese hamster ovary
CR Complete Response
DFS Disease free survival
DMSO Dimethylsulphoxide
DTR Dose limiting toxicity
FBS Fetal bovine serum
FFPE Formalin-fixed, paraffin-embedded
FR Framework region
IgG Immunoglobulin G
IHC Immunohistochemistry or immunohistochemical
MPK Milligram Per Kilogram (mg/kg or mg drug per kg body weight of animal)
MTD Maximum tolerated dose
NCBI National Center for Biotechnology Information
NCI National Cancer Institute
OR Overall response
OS Overall survival
PD Progressive disease
PFS Progression free survival
PR Partial response
Q2W One dose every two weeks
Q3W One dose every three weeks
Q4W One dose every four weeks
QD One dose per day
RECIST Response Evaluation Criteria in Solid Tumors
RPMI Roswell Park Memorial Institute
SD Stable disease
TGI Tumor Growth Inhibition
VH Immunoglobulin heavy chain variable region
VK Immunoglobulin kappa light chain variable region
w/w Weight per weight

### II. Definitions

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular form “a,” “an,” and “the” include plural references unless indicated otherwise. For example, “a” substituent includes one or more substituents. Where the plural form is used for compounds, salts, and the like, this is taken to mean also a single compound, salt, or the like.

The invention described herein suitably may be practiced in the absence of any element(s) not specifically disclosed herein. Thus, for example, in each instance herein any of the terms “comprising,” “consisting essentially of,” and “consisting of” may be replaced with either of the other two terms.

The term “about” when used to modify a numerically defined parameter (e.g., the dose of a CDK inhibitor, the dose of a PD-1 axis binding antagonist, the dose of an OX40 agonist (e.g., anti-OX40 antibody (αOX40)), the dose of a 4-1 BB agonist (e.g., anti-4-1 BB antibody (α4-BB)), and the like) means that the parameter may vary by as much as 10% above or below the stated numerical value for that parameter. For example, a dose of about 5 mg/kg should be understood to mean that the dose may vary between 4.5 mg/kg and 5.5 mg/kg.

As used herein, terms, including, but not limited to, “drug,” “agent,” “component,” “composition,” “compound,” “substance,” “targeted agent,” “targeted therapeutic agent,” “therapeutic agent,” and “medicament” may be used interchangeably to refer to the small molecule compounds of the present invention, e.g., a CDK inhibitor. As used herein, terms, including, but not limited to, “drug,” “agent,” “component,” “composition,” “compound,” “substance,” “targeted agent,” “targeted therapeutic agent,” “therapeutic agent,” therapeutic antibody,” and “medicament” may be used interchangeably to refer to the antibodies of the present invention, e.g., an anti-PD-L1 antibody, an anti-PD-1 antibody, an anti-OX40 antibody, and an anti-4-1BB antibody, or combinations thereof.

The term “therapeutic antibody” refers to an antibody that is used in the treatment of a disease or a disorder. A therapeutic antibody may have various mechanisms of action. A therapeutic antibody may bind and neutralize the normal function of a target associated with an antigen. For example, a monoclonal antibody that blocks the activity of the protein needed for the survival of a cancer cell causes the cell's death. Another therapeutic antibody may bind and activate the normal function of a target associated with an antigen. For example, a monoclonal antibody can bind to a protein on a cell and trigger an apoptosis signal. Yet another monoclonal antibody may bind to a target antigen expressed only on diseased tissue; conjugation of a toxic payload (effective agent), such as a chemotherapeutic or radioactive agent, to the monoclonal antibody can create an agent for specific delivery of the toxic payload to the diseased tissue, reducing harm to healthy tissue. A “biologically functional fragment” of a therapeutic antibody will exhibit at least one if not some or all of the biological functions attributed to the intact antibody, the function comprising at least specific binding to the target antigen.

The therapeutic antibody may bind to any protein, including, without limitation, a PD-L1, a PD-1, an OX40, and/or a 4-1BB antigen. Accordingly, therapeutic antibodies include, without limitation, anti-PD-L1 antibodies, anti-PD-1 antibodies, anti-OX40 antibodies, and anti-4-1BB antibodies, or combinations thereof.

“Biotherapeutic agent” means a biological molecule, such as an antibody or fusion protein, that blocks ligand/receptor signaling in any biological pathway that supports tumor maintenance and/or growth or suppresses the anti-tumor immune response.

A “chemotherapeutic agent” is a chemical compound useful in the treatment of cancer. Examples of chemotherapeutic agents include alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB 1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma and calicheamicin omegal (e.g., Nicolaou et al., Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HC1 liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c- it inhibitors; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDIS1NE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel)(TAXOTERE®; chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine)(NAVELBINE®); novantrone; edatrexate; daunomycin; aminopterin; ibandronate; topoisomerase inhibitor RFS 2000; difluorometlhylomithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Additional examples of chemotherapeutic agents include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY 1 1 7018, onapristone, and toremifene (FARESTON®); anti-progesterones; estrogen receptor down-regulators (ERDs); estrogen receptor antagonists such as fulvestrant) (FASLODEX®); agents that function to suppress or shut down the ovaries, for example, luteinizing hormone-releasing hormone (LHRFI) agonists such as leuprolide acetate (LUPRON® and ELIGARD®), goserelin acetate, buserelin acetate and tripterelin; anti-androgens such as fiutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RJVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1 ,3-dioxolane nucleoside cytosine analog); anti-sense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID vaccine; topoisomerase 1 inhibitor (e.g., LURTOTECAN®); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX®); lapatinib and lapatinib ditosylate (an ErbB-2 and EGFR dual tyrosine kinase small molecule inhibitor also known as GW572016); 17AAG (geldanamycin derivative that is a heat shock protein (Hsp) 90 poison), and pharmaceutically acceptable salts, acids or derivatives of any of the above.

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL- 1, IL-Ia, IL-2, IL-3, IL-4, IL-5, IL-6, IL-7, IL-8, IL-9, IL10, IL-11, IL-12, IL-13, IL-15, IL-17A-F, IL-18 to IL-29 (such as IL-23), IL-31 , including PROLEUKIN rIL-2; a tumor-necrosis factor such as TNF-a or TNF-β, TGF-I-3; and other polypeptide factors including leukemia inhibitory factor (“LIF”), ciliary neurotrophic factor (“CNTF”), CNTF-like cytokine (“CLC”), cardiotrophin (“CT”), and kit ligand (“L”).

As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Example chemokines include IL-8, a human homolog of murine keratinocyte chemoattractant (KC).

The terms “abnormal cell growth” and “hyperproliferative disorder” are used interchangeably in this application. “Abnormal cell growth,” as used herein, unless otherwise indicated, refers to cell growth that is independent of normal regulatory mechanisms (e.g., loss of contact inhibition). Abnormal cell growth may be benign (not cancerous), or malignant (cancerous).

A “disorder” is any condition that would benefit from treatment with the compounds of the present invention. This includes chronic and acute disorders or diseases including those pathological conditions which predispose the subject to the disorder in question.

The term “antibody,” as used herein, refers to an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses a polyclonal antibody, a monoclonal antibody, a chimeric antibody, a bispecific antibody, a dual-specific antibody, bifunctional antibody, a trispecific antibody, a multispecific antibody, a bispecific heterodimeric diabody, a bispecific heterodimeric IgG, a labeled antibody, a humanized antibody, a human antibody, and fragments thereof (such as Fab, Fab′, F(ab′)₂, Fv), single chain (ScFv) and domain antibodies (including, for example, shark and camelid antibodies), fusion proteins comprising an antibody, any other modified configuration of the immunoglobulin molecule that comprises an antigen recognition site, and antibody like binding peptidomimetics (ABiPs). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG-1, IgG-2, IgG-3, IgG-4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

As used herein, a “bispecific antibody,” “dual-specific antibody,” “bifunctional antibody,” “heteromultimer,” “heteromultimeric complex,” “bispecific heterodimeric diabody” or a “heteromultimeric polypeptide” is a molecule comprising at least a first polypeptide and a second polypeptide, wherein the second polypeptide differs in amino acid sequence from the first polypeptide by at least one amino acid residue. In some instances, the bispecific is an artificial hybrid antibody having two different heavy chain region and light chain region. Preferably, the bispecific antibody has binding specificity for at least two different ligands, antigens or binding sites. Accordingly, the bispecific antibodies can bind simultaneously two different antigens. The two antigen binding sites of a bispecific antibody bind to two different epitopes, which may reside on the same or different protein targets, e.g., tumor target.

The bispecific antibody, dual-specific antibody, bifunctional antibody, heteromultimer, heteromultimeric complex, bispecific heterodimeric diabody or the heteromultimeric polypeptide can be prepared by constructing sFv fragments with short linkers (e.g., about 3-10 residues) between the VH and VL regions such that inter-chain but not intra-chain pairing of the V regions is achieved, resulting in a bivalent fragment, i.e., fragment having two antigen-binding sites. Bispecific antibodies can be derived from full length antibodies or antibody fragments (e.g., F(ab′)₂ bispecific antibodies). Diabodies are described more fully in, for example, EP404,097; WO 1993/011161; and Hollinger et al., A small bispecific antibody construct expressed as a functional single-chain molecule with high tumor cell cytotoxicity, Proc. Natl. Acad. Sci. 1993, 90:6444-6448. Bispecific antibodies are heterodimers of two “crossover” sFv fragments in which the VH and VL regions of the two antibodies are present on different polypeptide chains.

By way of non-limiting example, a bispecific antibody may comprise one antigen-binding site that recognizes an epitope on one protein (e.g., OX40, 4-1BB, PD-1 or PD-L1) and further comprise a second, different antigen-binding site that recognizes a different epitope on a second protein (e.g., OX40, 4-1BB, PD-1 or PD-L1). Generally, but not necessarily, reference to binding means specific binding.

The term “immunoglobulin” (Ig) is used interchangeably with “antibody” herein. The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the a and γ chains and four CH domains for μ and ε isotypes. Each L chain has at the N-terminus, a variable domain (VL) followed by a constant domain at its other end. The VL is aligned with the VH and the CL is aligned with the first constant domain of the heavy chain (CHI). Particular amino acid residues are believed to form an interface between the light chain and heavy chain variable domains. The pairing of a VH and VL together forms a single antigen-binding site. For the structure and properties of the different classes of antibodies, e.g., Daniel P. Sties, Abba I. Terr and Tristram G. Parsolw (eds), Basic and Clinical Immunology, 8th Edition, 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes.

The terms “full-length antibody,” “intact antibody” or “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments suitable for use in this invention include, without limitation: (i) the Fab fragment, consisting of VL, VH, CL, and CH1 domains; (ii) the “Fd” fragment consisting of the VH and CH1 domains; (iii) the “Fv” fragment consisting of the VL and VH domains of a single antibody; (iv) the “dAb” fragment, which consists of a VH domain; (v) isolated CDR regions; (vi) F(ab')2 fragments, a bivalent fragment comprising two linked Fab fragments; (vii) single chain Fv molecules (scFv), wherein a VH domain and a VL domain are linked by a peptide linker that allows the two domains to associate to form a binding domain; (viii) bi-specific single chain Fv dimers (e.g., U.S. Pat. No. 5,091,513); and (ix) diabodies, multivalent or multispecific fragments constructed by gene fusion (US Patent App. Pub. 2005/0214860). Fv, scFv, or diabody molecules may be stabilized by the incorporation of disulphide bridges linking the VH and VL domains. Minibodies comprising a scFv joined to a CH3 domain may also be made (Hu et al., Minibodies are minimized antibody-like proteins comprising a scFv joined to a CH3 domain, Cancer Res. 1996, 56:3055-3061)).

Murali et al., Antibody like peptidomimetics as large scale immunodetection probes, Cell Mol Biol 2003, 49:209-216, describe a methodology for reducing antibodies into smaller peptidomimetics, they term “antibody like binding peptidomimetics” (ABiP) which may also be useful as an alternative to antibodies.

“Isolated antibody” or “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab,” as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al., Continuous cultures of fused cells secreting antibody of predefined specificity, Nature 1975, 256: 495; or may be made by recombinant DNA methods (e.g., U.S. Pat. No. 4,816,567). The “monoclonal antibodies” may also be isolated from phage antibody libraries using the techniques described in Clackson et al., Making antibody fragments using phage display libraries, Nature 1991, 352: 624-628 and Marks et al., By-passing immunization: human antibodies from V-gene libraries displayed on phage, J. Mol. Biol. 1991, 222: 581-597, for example. See also Presta, Selection, design, and engineering of therapeutic antibodies, J. Allergy Clin. Immunol. 2005, 116:731.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions.

The term “hypervariable region,” “HVR,” or “HV” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al, Disruption of Early Tumor Necrosis Factor Alpha Signaling Prevents Classical Activation of Dendritic Cells in Lung-Associated Lymph Nodes and Development of Protective Immunity against Cryptococcal Infection, Immunity 2000, J-3:37-45; Johnson and Wu, Antibody Engineering Methods and Protocols Methods in Molecular Biology 2003, 248: 1-25. Indeed, naturally occurring camelid antibodies consisting of a heavy chain only are functional and stable in the absence of light chain. See, e.g., Hamers-Casterman et al., Naturally occurring antibodies devoid of light chains, Nature 1993, 363:446-448; Sheriff et al., Similarity between C2 domain jaws and immunoglobulin CDRs, Nature Struct. Biol 1996, 3:733-736.

A number of HVR delineations are in use and are encompassed herein. The Kabat Complementarity Determining Regions (CDRs) are based on sequence variability and are the most commonly used (Kabat et al., Sequences of Proteins of Immunological

Interest, 5th Ed. Public Health Service, National Institutes of Health, 1991). Chothia refers instead to the location of the structural loops (Chothia and Lesk, Canonical structures for the hypervariable regions of immunoglobulins, J. Mol. Biol. 1987, 196:901 -917). The AbM HVRs represent a compromise between the Kabat HVRs and Chothia structural loops, are used by Oxford Molecular's AbM antibody modeling software. The “contact” HVRs are based on an analysis of the available complex crystal structures.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., Sequences of Proteins of Immunological Interest, 5th ed., Public Health Service, NIH, 1992. The positions of the CDRs may also be identified as the structural loop structures originally described by Chothia and others. See, e.g., Chothia et al., Conformations of immunoglobulin hypervariable regions, Nature, 1989 342:877-883. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., Antibody-antigen interactions: contact analysis and binding site topography, J. Mol. Biol., 1996, 262:732-745. In another approach, referred to herein as the “conformational definition” of CDRs, the positions of the CDRs may be identified as the residues that make enthalpic contributions to antigen binding. See, e.g., Makabe et al., Thermodynamic consequences of mutations in vernier zone residues of a humanized anti-human epidermal growth factor receptor murine antibody, 528, Journal of Biological Chemistry, 2008, 283:1156-1166. Still other CDR boundary definitions may not strictly follow one of the above approaches but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

“Framework” or “FR” residues are those variable-domain residues other than the HVR residues as herein defined.

A “human consensus framework” or “acceptor human framework” is a framework that represents the most commonly occurring amino acid residues in a selection of human immunoglobulin VL or VH framework sequences. Generally, the selection of human immunoglobulin VL or VH sequences is from a subgroup of variable domain sequences.

Generally, the subgroup of sequences is a subgroup as in Kabat et al., Sequences of Proteins of Immunological Interest, 5^{th} Ed. Public Health Service, National Institutes of Health, 1991. Examples for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra. Alternatively, a human consensus framework can be derived from the above in which particular residues, such as when a human framework residue is selected based on its homology to the donor framework by aligning the donor framework sequence with a collection of various human framework sequences. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

An “amino-acid modification” at a specified position, e.g., of the Fc region, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (e.g., Watson et al., Molecular Biology of the Gene (4th Ed.), 1987, p. 224). In addition, substitutions of structurally or functionally similar amino acids are less likely to disrupt biological activity. Exemplary conservative substitutions are set forth in Table 1 below.

TABLE 1

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

An “affinity-matured” antibody is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., By-passing immunization: Building high affinity human antibodies by chain shuffling, Bio/Technology 1992, 10:779-783, describes affinity maturation by VH- and VL-domain shuffling. Random mutagenesis of HVR and/or framework residues is described by, for example: Barbas et al., In vitro evolution of a neutralizing human antibody to human immunodeficiency virus type 1 to enhance affinity and broaden strain cross-reactivity, Proc Nat. Acad. Sci. 1994, 91:3809-3813; Schier et al., Identification of functional and structural amino-acid residues by parsimonious mutagenesis, Gene 1995, 169: 147-155; Yelton et al., Affinity maturation of the BR96 anti-carcinoma antibody by codon-based mutagenesis, J. Immunol. 1995, 155: 1994-2004; Jackson et al., In vitro antibody maturation. Improvement of a high affinity, neutralizing antibody against IL-1 beta, J. Immunol. 1995, 154(7):33 10-9; and Hawkins et al., Selection of phage antibodies by binding affinity: mimicking affinity maturation, J. Mol. Biol. 1992, 226:889-896.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof.

The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the invention include human IgG-1, IgG-2 (IgG2A, IgG2B), IgG-3 and IgG-4.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcyRI, FcyRII, and FeyRIII subclasses, including allelic variants and alternatively spliced forms of these receptors, FcyRII receptors include FcyRIIA (an “activating receptor”) and FcyRIIB (an “inhibiting receptor”), which have similar amino acid sequences that differ primarily in the cytoplasmic domains thereof. Activating receptor FcyRIIA contains an immunoreceptor tyrosine-based activation motif (ITAM) in its cytoplasmic domain. Inhibiting receptor FcyRIIB contains an immunoreceptor tyrosine-based inhibition motif (IT┌M) in its cytoplasmic domain, (e.g., M. Daeron, Fc RECEPTOR BIOLOGY, Annu. Rev. Immunol. J 1997, 5:203-234. FcRs are reviewed in Ravetch and Kinet, Fc receptors, Annu. Rev. Immunol. 1991, 9: 457-92; Capel et al., Heterogeneity of human IgG Fc receptors, Immunomethods 1994, 4: 25-34; and de Haas et al., Fcγ receptors of phagocytes, J. Lab. Clin. Med. 1995, 126: 330-41. Other FcRs, including those to be identified in the future, are encompassed by the term “FcR” herein.

The term Fc receptor or FcR also includes the neonatal receptor, FcRn, which is responsible for the transfer of maternal IgGs to the fetus. Guyer et al., Immunoglobulin binding by mouse intestinal epithelial cell receptors, J. Immunol. 1976, 1 17: 587, and Tokoyama et al., How do natural killer cells find self to achieve tolerance? Immunity, 1994, 24, 249-257. Methods of measuring binding to FcRn are known (e.g., Ghetie and Ward, FcRn: the MHC class I-related receptor that is more than an IgG transporter, Immunol. Today 1997, 1 8: (12): 592-8; Ghetie et al., Increasing the serum persistence of an IgG fragment by random mutagenesis, Nat Biotechnol. July 1997; 15(7):637-40; Hinton et al., Engineered human IgG antibodies with longer serum half-lives in primates, J. Biol. Chem. 2004, 279 (8): 6213-6; WO 2004/092219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/042072 (Presta) describes antibody variants which improved or diminished binding to FcRs. See also, e.g., Shields et al., High Resolution Mapping of the Binding Site on Human IgG1 for FcγRI, FcγRII, FcγRIII, and FcRn and Design of IgG1 Variants with Improved Binding to the FcγR, J. Biol. Chem. 2001, 9(2): 6591 -6604.

The phrase “substantially reduced,” “substantially different,” or “substantially inhibit,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

As use herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10 percent of the binding of the antibody to the target as measured, e.g., by a radioimmunoassay (RIA). In certain embodiments, an antibody that specifically binds to a target has a dissociation constant (Kd) of ≤1 ƒM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2 (including IgG2A and IgG2B), IgG-3, or IgG-4 subtypes, IgA (including IgA-1 and IgA-2), IgE, IgD or IgM. The Ig fusions preferably include the substitution of a domain of a polypeptide or antibody described herein in the place of at least one variable region within an Ig molecule. In a particularly preferred embodiment, the immunoglobulin fusion includes the hinge, CH2 and CH3, or the hinge, CHI, CH2 and CH3 regions of an IgG-1 molecule. For the production of immunoglobulin fusions, see also U.S. Pat. No. 5,428,130 issued June 27, 1995. Immunoadhesin combinations of Ig Fc and ECD of cell surface receptors are sometimes termed soluble receptors.

A “fusion protein” and a “fusion polypeptide” refer to a polypeptide having two portions covalently linked together, where each of the portions is a polypeptide having a different property. The property may be a biological property, such as activity in vitro or in vivo. The property may also be simple chemical or physical property, such as binding to a target molecule, catalysis of a reaction, etc. The two portions may be linked directly by a single peptide bond or through a peptide linker but are in reading frame with each other.

A “PD-1 oligopeptide,” “PD-L1 oligopeptide,” or “PD-L2 oligopeptide” is an oligopeptide that binds, preferably specifically, to a PD-1, PD-L1 or PD-L2 negative costimulatory polypeptide, respectively, including a receptor, ligand or signaling component, respectively, as described herein. Such oligopeptides may be chemically synthesized using known oligopeptide synthesis methodology or may be prepared and purified using recombinant technology. Such oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more. Such oligopeptides may be identified using well known techniques. In this regard, it is noted that techniques for screening oligopeptide libraries for oligopeptides that are capable of specifically binding to a polypeptide target are well known in the art (e.g., U.S. Pat. Nos. 5,556,762, 5,750,373, 4,708,871, 4,833,092, 5,223,409, 5,403,484, 5,571,689, 5,663,143; PCT Publication Nos. WO 1984/0003506 and WO 1984/0003564; Geysen et al., Use of peptide synthesis to probe viral antigens for epitopes to a resolution of a single amino acid, Proc. Natl. Acad. Sci. 1984, 81:3998-4002; Geysen et al., Small peptides induce antibodies with a sequence and structural requirement for binding antigen comparable to antibodies raised against the native protein, Proc. Natl. Acad. Sci. 1985, 82:178-182; Geysen et al., A priori delineation of a peptide which mimics a discontinuous antigenic determinant, Synthetic Peptides as Antigens, 1986, 130-149; Geysen, et al., Strategies for epitope analysis using peptide synthesis, J. Immunol. Meth. 1987, 102, 259-274; Schoofs et al., Epitopes of an influenza viral peptide recognized by antibody at single amino acid resolution, J. Immunol, 1988, 140:611-616, Cwirla, S. E. et al., Peptides on phage: a vast library of peptides for identifying ligands., Proc. Natl. Acad. Sci. 1990, 87:6378; Lowman, H. B. et al., Selecting high-affinity binding proteins by monovalent phage display, Biochemistry, 1991, 30:10832; Clackson, T. et al., Making antibody fragments using phage display libraries, Nature, 1991, 352: 624; Marks, J. D. et al., By-passing immunization: human antibodies from V-gene libraries displayed on phage, J. Mol. Biol, 1991, 222:581; Kang, et al., Linkage of Recognition and Replication Functions by Assembling Combinatorial Antibody Fab Libraries Along Phage Surfaces, PNAS, 1991, vol. 88, pp. 4363-4366, and Smith, G. P. Surface presentation of protein epitopes using bacteriophage expression systems, Curr. Opin. Biotechnol. 1991, 2:668.

An “antagonist” antibody or a “blocking” antibody is one that inhibits or reduces a biological activity of the antigen it binds. In some embodiments, blocking antibodies or antagonist antibodies substantially or completely inhibit the biological activity of the antigen. The anti-PD-L1 antibodies of the invention block the signaling through PD-1 so as to restore a functional response by T-cells (e.g., proliferation, cytokine production, target cell killing) from a dysfunctional state to antigen stimulation.

An “agonist” or “activating antibody” is one that enhances or initiates signaling by the antigen to which it binds. In some embodiments, agonist antibodies cause or activate signaling without the presence of the natural ligand.

The term “dysfunction” in the context of immune dysfunction, refers to a state of reduced immune responsiveness to antigenic stimulation. The term includes the common elements of both exhaustion and/or anergy in which antigen recognition may occur, but the ensuing immune response is ineffective to control infection or tumor growth.

The term “dysfunctional”, as used herein, also includes refractory or unresponsive to antigen recognition, specifically, impaired capacity to translate antigen recognition into down-stream T-cell effector functions, such as proliferation, cytokine production and/or target cell killing.

The term “anergy” refers to the state of unresponsiveness to antigen stimulation resulting from incomplete or insufficient signals delivered through the T-cell receptor (e.g., increase in intracellular Ca+2 in the absence of ras-activation). T cell anergy can also result upon stimulation with antigen in the absence of co- stimulation, resulting in the cell becoming refractory to subsequent activation by the antigen even in the context of co stimulation. The unresponsive state can often be overridden by the presence of Interleukin-2. Anergic T-cells do not undergo clonal expansion and/or acquire effector functions.

The term “exhaustion” refers to T cell exhaustion as a state of T cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that of functional effector or memory T cells. Exhaustion prevents optimal control of infection and tumors. Exhaustion can result from both extrinsic negative regulatory pathways (e.g., immunoregulatory cytokines) as well as cell intrinsic negative regulatory (co stimulatory) pathways.

“Enhancing T-cell function” means to induce, cause or stimulate a T-cell to have a sustained or amplified biological function, or renew or reactivate exhausted or dysfunctional T-cells. Examples of enhancing T-cell function include: increased secretion of y-interferon from CD4+ or CD8+ T-cells, increased proliferation, increased survival, increased differentiation, increased antigen responsiveness (e.g., viral, pathogen, or tumor clearance) relative to such levels before the intervention. In some embodiments, the level of enhancement is as least 50%, alternatively 60%, 70%, 80%, 90%, 100%, 120%, 150%, 200%. The manner of measuring this enhancement is known to one of ordinary skill in the art.

As used herein, “metastasis” or “metastatic” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

The term “cancer,” “cancerous,” or “malignant” refers to or describe the physiological condition in subjects that is typically characterized by unregulated cell growth. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type from the latter one. Examples of cancer include, but are not limited to, brain cancer, head/neck cancer (including squamous cell carcinoma of the head and neck (SCCHN)), prostate cancer, ovarian cancer, bladder cancer (including urothelial carcinoma, also known as transitional cell carcinoma (TCC)), lung cancer (including squamous cell carcinoma, small cell lung cancer (SCLC), and non-small cell lung cancer (NSCLC)), breast cancer, bone cancer, colorectal cancer, kidney cancer, liver cancer (including hepatocellular carcinoma (HCC)), stomach cancer, pancreatic cancer, esophageal cancer , cervical cancer, sarcoma, skin cancer (including melanoma and Merkel cell carcinoma (MCC)), multiple myeloma, mesothelioma, malignant rhabdoid tumors, diffuse intrinsic pontine glioma (DIPG), carcinoma, lymphoma, diffuse large B-cell lymphoma (DLBCL), primary mediastinal B-cell lymphoma (PMBCL), follicular lymphoma, acute lymphoblastic leukemia (ALL), acute myeloid leukemia (AML), chronic lymphocytic leukemia (CLL), chronic myeloid leukemia (CML), follicular lymphoma, Hodgkin's lymphoma (HL), classical Hodgkin lymphoma (cHL), mantle cell lymphoma (MCL), multiple myeloma (MM), myeloid cell leukemia-1 protein (Mcl-1), myelodysplastic syndrome (MDS), non-Hodgkin's lymphoma (NHL), small lymphocytic lymphoma (SLL), and SWI/SNF-mutant cancer.

As used herein, “in combination with” or “in conjunction with” refers to administration of one treatment modality in addition to at least one other treatment modality. As such, “in combination with” or “in conjunction with” refers to administration of one treatment modality before, during, or after administration of at least one other treatment modality to the individual.

An “objective response” refers to a measurable response, including complete response (CR) or partial response (PR). In some embodiments, the term “objective response rate” (ORR) refers to the sum of complete response (CR) rate and partial response (PR) rate.

“Complete response” or “CR,” as used herein, means the disappearance of all signs of cancer (e.g., disappearance of all target lesions) in response to treatment. This does not always mean the cancer has been cured.

As used herein, “partial response” or “PR” refers to a decrease in the size of one or more tumors or lesions, or in the extent of cancer in the body, in response to treatment. For example, in some embodiments, PR refers to at least a 30% decrease in the sum of the longest diameters (SLD) of target lesions, taking as reference the baseline SLD.

As used herein, “progressive disease” or “PD” refers to at least a 20% increase in the SLD of target lesions, taking as reference the smallest SLD recorded since the treatment started or the presence of one or more new lesions.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” (ORR) refers to the sum of complete response (CR) rate and partial response (PR) rate.

As used herein, “overall survival” refers to the percentage of individuals in a group who are likely to be alive after a particular duration of time.

“Sustained response” refers to the sustained effect on reducing tumor growth after cessation of a treatment. For example, the tumor size may be the same size or smaller as compared to the size at the beginning of the medicament administration phase. In some embodiments, the sustained response has a duration of at least the same as the treatment duration, at least 1.5×, 2×, 2.5×, or 3× length of the treatment duration, or longer.

“Duration of Response” for purposes of the present invention means the time from documentation of tumor model growth inhibition due to drug treatment to the time of acquisition of a restored growth rate similar to pretreatment growth rate.

In some embodiments, the anti-cancer effect of the method of treating cancer, including “objective response,” “complete response,” “partial response,” “progressive disease,” “stable disease,” “progression free survival,” “duration of response,” as used herein, are as defined and assessed by the investigators using RECIST v1.1 (Eisenhauer et al., New response evaluation criteria in solid tumors: revised RECIST guideline, Eur J of Cancer 2009; 45(2):228-47) in patients with locally advanced or metastatic solid tumors other than metastatic CRPC, and RECIST v1.1 and PCWG3 (Scher et al., Trial Design and Objectives for Castration-Resistant Prostate Cancer: Updated Recommendations From the Prostate Cancer Clinical Trials Working Group 3, J Clin Oncol 2016; 34(12):1402-18) in patients with metastatic CRPC. The disclosures of Eisenhauer et al., 2009 and Scher et al., 2016 are herein incorporated by references in their entireties.

The term “patient” or “subject” refers to any subject for which therapy is desired or that is participating in a clinical trial, epidemiological study or used as a control, including humans and non-human animals, including veterinary subjects such as cattle, horses, dogs and cats. In a preferred embodiment, the subject is a human and may be referred to as a patient. Those skilled in the medical art are readily able to identify individual patients who are afflicted with cancer.

In some embodiments, the combination or co-administration of two or more agents can be useful for treating individuals suffering from cancer who have primary or acquired resistance to ongoing therapies. The combination therapy provided herein may be useful for improving the efficacy and/or reducing the side effects of cancer therapies for individuals who do respond to such therapies.

As used herein, the term “combination therapy” refers to the administration of each agent of the combination therapy of the invention, either alone or in a medicament, either simultaneously, separately or sequentially, as mixed or individual dosages.

As used herein, the term “simultaneously,” “simultaneous administration,” “administered simultaneously,” “concurrently,” or “concurrent administration,” means that the agents are administered at the same point in time or immediately following one another, but that the agents can be administered in any order. For example, in the latter case, the two or more agents are administered at times sufficiently close that the results observed are indistinguishable from those achieved when the agents are administered at the same point in time. The term simultaneous includes the administration of each agent of the combination therapy of the invention in the same medicament.

The agents of the present invention can be administered completely separately or in the form of one or more separate compositions. For example, the agents may be given separately at different times during the course of therapy (in a chronologically staggered manner, especially a sequence-specific manner) in such time intervals that the combination therapy is effective in treating cancer.

As used herein, the term “sequential,” “sequentially,” “administered sequentially,” or “sequential administration” refers to the administration of each agent of the combination therapy of the invention, either alone or in a medicament, one after the other, wherein each agent can be administered in any order. Sequential administration may be particularly useful when the therapeutic agents in the combination therapy are in different dosage forms, for example, one agent is a tablet and another agent is a sterile liquid, and/or the agents are administered according to different dosing schedules, for example, one agent is administered daily, and the second agent is administered less frequently such as weekly.

As used herein, “in combination with,” “in conjunction with” or “combined administration” refers to administration of one agent in addition to at least one other agent. As such, “in combination with,” “in conjunction with” or “combined administration” refers to administration of one agent before, during, or after administration of at least one other agent to the individual. The administration of two or more agents are intended to include treatment regimens in which the agents are not necessarily administered by the same route of administration or at the same time.

A “combination” or “pharmaceutical combination” refers to a combination of any two or more agents as described herein, e.g., any CDK inhibitor described herein with any PD-1 axis binding antagonist as described herein, optionally with any OX40 agonist as described herein; any 4-1 BB agonist as described herein; or any OX40 agonist and any 4-1 BB agonist as described herein. These two or more agents may (but do not necessarily) belong to different classes of agents.

In some embodiments, a combination as described herein, e.g., a CDK inhibitor in combination with a PD-1 axis binding antagonist, is administered in a single dose. In some embodiments, a combination as described herein, e.g., a CDK inhibitor in combination with a PD-1 axis binding antagonist, is administered in multiple doses. In some embodiments, an amount of a combination as described herein, e.g., a CDK inhibitor in combination with a PD-1 axis binding antagonist, may be administered periodically at regular intervals (e.g., 1, 2, 3, 4, 5, 6, 7, 8, 9, 10 or more times every 1, 2, 3, 4, 5, or 6 days, or every 1, 2, 3, 4, 5, 6, 7, 8, or 9 weeks, or every 1, 2, 3, 4, 5, 6, 7, 8, 9 months or longer).

In some embodiments, a combination as described herein, e.g., a CDK inhibitor in combination with a PD-1 axis binding antagonist, is administered at a predetermined interval (e.g., 1, 2, 3, 4, 5, 6, 7, 8, 9, 10 or more times every 1, 2, 3, 4, 5, or 6 days, or every 1, 2, 3, 4, 5, 6, 7, 8, or 9 weeks, or every 1, 2, 3, 4, 5, 6, 7, 8, 9 months or longer).

The present invention relates to combinations of two or more agents for simultaneous, separate or sequential administration, in particular for the treatment or prevention of cancer. For example, the individual agents of the combination of the invention can be administered separately at different times in any order during the course of therapy or concurrently in divided or single combination forms.

The terms “concurrent administration,” “administration in combination,” “simultaneous administration” or “administered simultaneously,” as used herein, means that the agents are administered at the same point in time or immediately following one another. For example, in the latter case, the two agents are administered at times sufficiently close that the results observed are indistinguishable from those achieved when the agents are administered at the same point in time.

The agents of the present invention can be administered completely separately or in the form of one or more separate compositions. For example, the agents may be given separately at different times during the course of therapy (in a chronologically staggered manner, especially a sequence-specific manner) in such time intervals that the combination therapy is effective in treating cancer.

The term “sequentially,” as used herein, refers to a treatment in which administration of a first treatment, such as administration of first agent, follows administration of a second treatment, such as administration of a second agent.

The dosage of the individual agents of the combination may require more frequent administration of one of the agent(s) as compared to the other agent(s) in the combination. Therefore, to permit appropriate dosing, packaged pharmaceutical products may contain one or more dosage forms that contain the combination of agents, and one or more dosage forms that contain one of the combination of agents, but not the other agent(s) of the combination.

The term “single formulation,” as used herein, refers to a single carrier or vehicle formulated to deliver effective amounts of both therapeutic agents to a subject. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension.

The term “unit dose” is used herein to mean simultaneous administration of both agents together, in one dosage form, to the subject being treated. In some embodiments, the unit dose is a single formulation. In certain embodiments, the unit dose includes one or more vehicles such that each vehicle includes an effective amount of at least one of the agents along with pharmaceutically acceptable carriers and excipients. In some embodiments, the unit dose is one or more tablets, capsules, pills, or patches administered to the subject at the same time.

An “oral dosage form” includes a unit dosage form prescribed or intended for oral administration.

The term “advanced,” as used herein, as it relates to breast cancer, includes locally advanced (non-metastatic) disease and metastatic disease.

The term “treat” or “treating” a cancer, as used herein means to administer a combination therapy according to the present invention to a subject having cancer, or diagnosed with cancer, to achieve at least one positive therapeutic effect, such as, for example, reduced number of cancer cells, reduced tumor size, reduced rate of cancer cell infiltration into peripheral organize, or reduced rate of tumor metastases or tumor growth, reversing, stopping, controlling, slowing, interrupting, arresting, alleviating, and/or inhibiting the progression or severity of a sign, symptom, disorder, condition, or disease, but does not necessarily involve a total elimination of all disease-related signs, symptoms, conditions, or disorders. Within the meaning of the present invention, the term “treat” or “treating” also denotes, to arrest, delay the onset (i.e., the period prior to clinical manifestation of a disease or symptom of a disease) and/or reduce the risk of developing or worsening a symptom of a disease.

The term “treatment,” as used herein, unless otherwise indicated, refers to the act of treating as “treating” is defined immediately above. The term “treating” also includes adjuvant and neo-adjuvant treatment of a subject. For the purposes of this invention, beneficial or desired clinical results include, but are not limited to, one or more of the following: reducing the proliferation of (or destroying) neoplastic or cancerous cell; inhibiting metastasis or neoplastic cells; shrinking or decreasing the size of tumor; remission of the cancer; decreasing at least one symptom resulting from the cancer; increasing the quality of life of those suffering from the cancer; decreasing the dose of other medications required to treat the cancer; delaying the progression the cancer;

curing the cancer; overcoming one or more resistance mechanisms of the cancer; and/or prolonging survival of patients with cancer. Positive therapeutic effects in cancer can be measured in a number of ways (e.g., W. A. Weber, J. Nucl. Med. 50:1S-10S (200)). In some embodiments, the treatment achieved by a combination of the invention is any of the partial response (PR), complete response (CR), overall response (OR), progression free survival (PFS), disease free survival (DFS) and overall survival (OS). PFS, also referred to as “Time to Tumor Progression” indicates the length of time during and after treatment that the cancer does not grow and includes the amount of time patients have experienced a CR or PR, as well as the amount of time patients have experienced stable disease (SD). DFS refers to the length of time during and after treatment that the patient remains free of disease. OS refers to a prolongation in life expectancy as compared to naïve or untreated subjects or patients. In some embodiments, response to a combination of the invention is any of PR, CR, OR, OS, PFS, or DFS that is assessed using Response Evaluation Criteria in Solid Tumors (RECIST) 1.1 response criteria. The treatment regimen for a combination of the invention that is effective to treat a cancer patient may vary according to factors such as the disease state, age, weight of the patient, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as the Student's t-test, the chi2-test the U-test according to Mann and Whitney, the Kruskal-Wallis test (H-test), Jonckheere-Terpstrat-testy and the Wilcon on-test.

The term “administer,” “administering,” or “administration,” “treat,” “treating,” or “treatment” as it applies to an animal, human, experimental subject, cell, tissue, organ or biological fluid, refers to contacting, implanting, absorbing, ingesting, injecting, inhaling, or introducing of an exogenous pharmaceutical, therapeutic or diagnostic agent, compound, particle, and/or composition, to the animal, human, experimental subject, cell, tissue, organ or biological fluid. Treatment of a cell encompasses contact of an agent to the cell, as well as contact of an agent to a fluid, where the fluid is in contact with the cell. The term “treatment” also encompasses in vitro and ex vivo treatment, e.g., of a cell, by a reagent, diagnostic, binding compound, or by another cell.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, e.g., by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain biomarkers in a biological sample from an individual.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

The term “pharmaceutical composition” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” carriers or excipients (vehicles, additives) are those which can reasonably be administered to a subject to provide an effective dose of the active ingredient employed.

The combinations provided herein may be formulated by a variety of methods apparent to those of skill in the art of pharmaceutical formulation. The various release properties described above may be achieved in a variety of different ways. Suitable formulations include, for example, tablets, capsules, press coat formulations, and other easily administered formulations.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

An “effective dosage,” “effective amount,” “therapeutically effective amount,” or “therapeutically effective dosage” of a drug, agent, component, composition, compound, substance, targeted agent, targeted therapeutic agent, therapeutic antibody, therapeutic agent, medicament or pharmaceutical composition is an amount to affect any one or more beneficial or desired, including biochemical, histological and/or behavioral, symptoms of the disease, its complications and intermediate pathological phenotypes presenting during development of the disease.

For therapeutic use, a therapeutically effective amount refers to that amount of a drug, agent, component, composition, compound, substance, targeted agent, targeted therapeutic agent, therapeutic antibody, therapeutic agent, medicament or pharmaceutical composition being administered which will relieve to some extent one or more of the symptoms of the disorder being treated such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival.

In reference to the treatment of cancer, a therapeutically effective amount refers to that amount of a drug, agent, component, composition, compound, substance, targeted agent, targeted therapeutic agent, therapeutic antibody, therapeutic agent, medicament or pharmaceutical composition which is effective to achieve one or more of the following results following the administration of one or more therapies: (1) reducing the size of the tumor, (2) reducing the number of cancer cells, (3) inhibiting (i.e., slowing to some extent, preferably stopping) cancer cell infiltration into peripheral organs, (4) inhibiting (i.e., slowing to some extent, preferably stopping) tumor metastasis, (5) inhibiting (i.e., slowing to some extent, preferably stopping) tumor growth or tumor invasiveness, (6) relieving (i.e., to some extent, preferably eliminating) one or more signs or symptoms associated with the cancer, (7) decreasing the dose of other medications required to treat the disease, (8) enhancing the effect of another medication, (9) delaying the progression of the disease, (10) improving or increasing the disease-free, relapse-free, progression-free, and/or overall survival, duration, or rate, (11) increasing the response rate, the durability of response, or number of patients who respond or are in remission, (12) decreasing the hospitalization rate, (13) decreasing the hospitalization lengths, (14) the size of the tumor is maintained and does not increase or increases by less than 10%, preferably less than 5%, preferably less than 4%, preferably less than 2%; (12) an increase in the number of patients in remission, (15) increasing the length or duration of remission, (16) decreasing the recurrence rate of cancer; (15) decreasing the time to recurrence of cancer, and (17) an amelioration of cancer related symptoms and/or quality of life.

In accordance with the present invention, an amount of a CDK inhibitor is combined with an amount of a PD-1 axis binding antagonist, and optionally amounts of an OX40 agonist and/or an amount of a 4-1 BB agonist, wherein the amounts together are effective in the treatment of cancer.

An effective amount can be administered in one or more administrations. For the purposes of this invention, an effective amount is an amount sufficient to accomplish prophylactic or therapeutic treatment either directly or indirectly. As is understood in the clinical context, an effective amount of a drug, compound or pharmaceutical composition may or may not be achieved in conjunction with another drug, agent, component, composition, compound, substance, targeted agent, targeted therapeutic agent, therapeutic antibody, therapeutic agent, medicament or pharmaceutical composition.

An effective amount can be administered in one or more administrations. For purposes of this invention, an effective amount of drug, compound, and/or pharmaceutical composition is an amount sufficient to accomplish prophylactic or therapeutic treatment either directly or indirectly.

As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

A therapeutic amount may also refer to a dosage of a drug that has been approved for use by a regulatory agency. A “subtherapeutic amount,” as used herein, refers to a dosage of a drug that is significantly lower than the approved dosage.

The terms “treatment regimen,” “dosing protocol” and “dosing regimen” are used interchangeably to refer to the dose and timing of administration of each therapeutic agent in a combination of the invention.

The term “ameliorating,” with reference to a disease, disorder or condition, refers to any observable beneficial effect of the treatment. Treatment need not be absolute to be beneficial to the subject. For example, ameliorating means a lessening or improvement of one or more symptoms of a disease, disorder or condition as compared to not administering a therapeutic agent of a method or regimen of the invention. Ameliorating also includes shortening or reduction in duration of a symptom.

The term “biosimilar” refers to a biological product that is highly similar to an FDA-approved biological product (reference product) and has no clinically meaningful differences in terms of pharmacokinetics, safety and efficacy from the reference product.

The term “bioequivalent” refers to a biological product that is pharmaceutically 5 equivalent and has a similar bioavailability to an FDA-approved biological product (reference product). For example, according to the FDA the term bioequivalence is defined as “the absence of a significant difference in the rate and extent to which the active ingredient or active moiety in pharmaceutical equivalents or pharmaceutical alternatives becomes available at the site of drug action when administered at the same molar dose under similar conditions 10 in an appropriately designed study” (United States Food and Drug Administration, “Guidance for Industry: Bioavailability and Bioequicalence Studies for Orally Administered Drug Products-General Considerations,” 2003, Center for Drug Evaluation and Research).

The term “biobetter” refers a biological product that is in the same class as an FDA approved biological product (reference product) but is not identical and is improved in terms 15 of safety, efficacy, stability, etc. over the reference product.

“Tumor” as it applies to a subject diagnosed with, or suspected of having, a cancer refers to a malignant or potentially malignant neoplasm or tissue mass of any size and includes primary tumors and secondary neoplasms. A solid tumor is an abnormal growth or mass of tissue that usually does not contain cysts or liquid areas. Examples of solid tumors are sarcomas, carcinomas, and lymphomas. Leukemia's (cancers of the blood) generally do not form solid tumors (National Cancer Institute, Dictionary of Cancer Terms).

“Tumor burden” also referred to as a “tumor load”, refers to the total amount of tumor material distributed throughout the body. Tumor burden refers to the total number of cancer cells or the total size of tumor(s), throughout the body, including lymph nodes and bone marrow. Tumor burden can be determined by a variety of methods known in the art, such as, e.g., using calipers, or while in the body using imaging techniques, e.g., ultrasound, bone scan, computed tomography (CT), or magnetic resonance imaging (MRI) scans.

The term “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CR or MRI scans.

The term “additive” is used to mean that the result of the combination of two or more agents is no greater than the sum of each agent individually.

In one embodiment, the combination of agents described herein displays a synergistic effect. The term “synergy” or “synergistic” are used to mean that the result of the combination of two or more agents is greater than the sum of each agent individually. This improvement in the disease, condition or disorder being treated is a “synergistic” effect. A “synergistic amount” is an amount of the combination of the two or more agents that results in a synergistic effect, as “synergistic” is defined herein. A “synergistic combination” refers to a combination of agents which produces a synergistic effect in vivo, or alternatively in vitro as measured according to the methods described herein.

Determining a synergistic interaction between two or more agents, the optimum range for the effect and absolute dose ranges of each agent for the effect may be definitively measured by administration of the agents over different dose ranges, and/or dose ratios to subjects in need of treatment. However, the observation of synergy in in vitro models or in vivo models can be predictive of the effect in humans and other species and in vitro models or in vivo models exist, as described herein, to measure a synergistic effect. The results of such studies can also be used to predict effective dose and plasma concentration ratio ranges and the absolute doses and plasma concentrations required in humans and other species such as by the application of pharmacokinetic and/or pharmacodynamics methods.

A “nonstandard clinical dosing regimen,” as used herein, refers to a regimen for administering a substance, agent, compound or composition, which is different to the amount, dose or schedule typically used for that substance, agent, compound or composition in a clinical setting. A “non-standard clinical dosing regimen,” includes a “non-standard clinical dose” or a “nonstandard dosing schedule”.

A “low dose amount regimen,” as used herein, refers to a dosing regimen where one or more of the substances, agents, compounds or compositions in the regimen are dosed at a lower amount or dose than typically used in a clinical setting for that agent, for example when that agent is dosed as a singleton therapy.

The term “pharmaceutically acceptable salt,” as used herein, refers to pharmaceutically acceptable organic or inorganic salts of a compound of the invention. Some embodiments also relate to the pharmaceutically acceptable acid addition salts of the compounds described herein. Suitable acid addition salts are formed from acids which form non-toxic salts. Non-limiting examples of suitable acid addition salts, i.e., salts containing pharmacologically acceptable anions, include, but are not limited to, the acetate, acid citrate, adipate, aspartate, benzoate, besylate, bicarbonate/carbonate, bisulphate/sulphate, bitartrate, borate, camsylate, citrate, cyclamate, edisylate, esylate, ethanesulfonate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, methanesulfonate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, p-toluenesulfonate, trifluoroacetate and xinofoate salts.

Additional embodiments relate to base addition salts of the compounds described herein. Suitable base addition salts are formed from bases which form non-toxic salts. Non-limiting examples of suitable base salts include the aluminum, arginine, benzathine, calcium, choline, diethylamine, diolamine, glycine, lysine, magnesium, meglumine, olamine, potassium, sodium, tromethamine and zinc salts.

The compounds described herein that are basic in nature are capable of forming a wide variety of salts with various inorganic and organic acids. The acids that may be used to prepare pharmaceutically acceptable acid addition salts of such basic compounds described herein are those that form non-toxic acid addition salts, e.g., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds described herein that include a basic moiety, such as an amino group, may form pharmaceutically acceptable salts with various amino acids, in addition to the acids mentioned above.

The chemical bases that may be used as reagents to prepare pharmaceutically acceptable base salts of those compounds described herein that are acidic in nature are those that form non-toxic base salts with such compounds. Such non-toxic base salts include but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines. Hem isalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds described herein are known to one of skill in the art.

“Carriers,” as used herein, include pharmaceutically acceptable carriers, excipients, or stabilizers that are nontoxic to the cell or subject being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “solvate” is used herein to describe a molecular complex comprising a compound described herein and one or more pharmaceutically acceptable solvent molecules, for example, water and ethanol.

The compounds described herein may also exist in unsolvated and solvated forms. Accordingly, some embodiments relate to the hydrates and solvates of the compounds described herein.

Compounds described herein containing one or more asymmetric carbon atoms can exist as two or more stereoisomers. Where a compound described herein contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds described herein containing, for example, an imino, keto, or oxime group, or so-called valence tautomerism in compounds which contain an aromatic moiety. A single compound may exhibit more than one type of isomerism.

The compounds of the embodiments described herein include all stereoisomers (e.g., cis and trans isomers) and all optical isomers of compounds described herein (e.g., R and S enantiomers), as well as racemic, diastereomeric and other mixtures of such isomers. While all stereoisomers are encompassed within the scope of our claims, one skilled in the art will recognize that particular stereoisomers may be preferred.

In some embodiments, the compounds described herein can exist in several tautomeric forms, including the enol and imine form, and the keto and enamine form and geometric isomers and mixtures thereof. All such tautomeric forms are included within the scope of the present embodiments. Tautomers exist as mixtures of a tautomeric set in solution. In solid form, usually one tautomer predominates. Even though one tautomer may be described, the present embodiments include all tautomers of the present compounds.

Included within the scope of the present embodiments are all stereoisomers, geometric isomers and tautomeric forms of the compounds described herein, including compounds exhibiting more than one type of isomerism, and mixtures of one or more thereof. Also included are acid addition or base salts wherein the counterion is optically active, for example, d-lactate or 1-lysine, or racemic, for example, dl-tartrate or dl-arginine.

The present embodiments also include atropisomers of the compounds described herein. Atropisomers refer to compounds that can be separated into rotationally restricted isomers.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC).

Alternatively, the racemate (or a racemic precursor) may be reacted with a suitable optically active compound, for example, an alcohol, or, in the case where a compound described herein contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Exemplary methods and materials are described herein, although methods and materials similar or equivalent to those described herein can also be used in the practice or testing of the invention. The materials, methods, and examples are illustrative only and not intended to be limiting.

### III. CDK Inhibitors

Embodiments of the present invention comprise a CDK inhibitor. CDKs and related serine/threonine kinases are important cellular enzymes that perform essential functions in regulating cell division and proliferation.

In an embodiment, the CDK inhibitor is an inhibitor of CDK4/6 (CDK4/6 inhibitor or CDK4/6i) or an inhibitor of CDK2/4/6 (CDK2/4/6 inhibitor or CDK2/4/6i).

In one such embodiment, the CDK2/4/6 inhibitor is 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one (PF-06873600), or a pharmaceutically acceptable salt thereof.

In another embodiment, the CDK4/6 inhibitor is palbociclib, or a pharmaceutically acceptable salt thereof. Palbociclib refers to 6-acetyl-8-cyclopentyl-5-methyl-2-(5-piperazin-1-yl-pyridin-2-ylamino)-8H-pyrido[2,3-d]pyrimidin-7-one, or a pharmaceutically acceptable salt thereof.

### IV. PD-1 Axis Binding Antagonists

Embodiments of the present invention comprise a PD-1 axis binding antagonist.

As used herein, the term “PD-1 axis binding antagonist” or “PD-1 axis antagonist” refers to a molecule that inhibits the interaction of a PD-1 axis binding partner (e.g., PD-1, PD-L1, PD-L2) with either one or more of its binding partners, for example so as to overcome or partially overcome T-cell dysfunction resulting from signaling on the PD-1 signaling axis—with a result being to restore, partially restore or enhance T-cell function (e.g., proliferation, cytokine production, target cell killing, survival). As used herein, a PD-1 axis binding antagonist includes one or more of (i) a PD-1 binding antagonist, (ii) a PD-L1 binding antagonist, and/or (iii) a PD-L2 antagonist.

The term “PD-1 binding antagonist,” as used herein, refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In some embodiments, a PD-1 binding antagonist reduces the negative co-stimulatory signal mediated by or through cell surface proteins expressed on T lymphocytes mediated signaling through PD-1 so as to render a dysfunctional T-cell less non-dysfunctional. In some embodiments, the PD-1 binding antagonist is an anti-PD-1 antibody (αPD-1). In some embodiments, a PD-1 binding antagonist is nivolumab. In some embodiments, a PD-1 binding antagonist is pembrolizumab. In some embodiments, a PD-1 binding antagonist is pidilizumab.

In some embodiments, the PD-1 binding antagonist useful for this invention is selected from the group consisting of MDX-1106 (nivolumab), MK-3475 (pembrolizumab), CT-011 (pidilizumab), MEDI-0680 (AMP-514), REGN-2810 (cemiplimab), mAb7 (RN888), mAb15, AMP-224 (B7-DCIg), and AGEN-2034w spartalizumab.

Exemplary PD-1 binding antagonists include those described in U.S. Patent Application Publication 20130280265, U.S. Patent Application Publication 20130237580, U.S. Patent Application Publication 20130230514, U.S. Patent Application Publication 20130109843, U.S. Patent Application Publication 20130108651, U.S. Patent Application Publication 20130017199, U.S. Patent Application Publication 20120251537, U.S. Patent Application Publication 20110271358, European Patent EP2170959B1, in PCT Publication No. WO 2011/066342, PCT Publication No. WO 2015/035606, PCT Publication No. WO 2015/085847, PCT Publication No. WO 2015/112800, PCT Publication No. WO 2015/112900, PCT Publication No. WO 2016/092419, PCT Publication No. WO 2017/017623, PCT Publication No. WO 2017/024465, PCT Publication No. WO 2017/054646, PCT Publication No. WO 2017/071625, PCT Publication No. WO 2017/019846, PCT Publication No. WO 2017/132827, PCT Publication No. WO 2017/214092, PCT Publication No. WO 2018/013017, PCT Publication No. WO 2018/053106, PCT Publication No. WO 2018/055503, PCT Publication No. WO 2018/053709, PCT Publication No. WO 2018/068336, and PCT Publication No. WO 2018/072743, the entire disclosures of which are incorporated herein by reference. Other exemplary PD-1 binding antagonists are described in Curran et al., PD-1 and CTLA-4 combination blockade expands infiltrating T cells and reduces regulatory T and myeloid cells within B16 melanoma tumors, PNAS, 2010, 107, 4275; Topalian et al., Safety, activity, and immune correlates of anti-PD-1 antibody in cancer, New Engl. J. Med. 2012, 366, 2443; Brahmer et al., Safety and activity of anti-PD-L1 antibody in patients with advanced cancer, New Engl. J. Med. 2012, 366, 2455; Dolan et al., PD-1 pathway inhibitors: changing the landscape of cancer immunotherapy, Cancer Control 2014, 21, 3; and Sunshine et al., Pd-1/Pd-L1 Inhibitors, Curr. Opin. in Pharmacol. 2015, 23.

The term “PD-L1 binding antagonist,” as used herein, refers to a molecule that decreases, blocks, inhibits, abrogates or interferes with signal transduction resulting from the interaction of PD-L1 with either one or more of its binding partners, such as PD-1, B7-1. In some embodiments, the PD-1 axis binding antagonist comprises a PD-L1 binding antagonist. In some embodiments, the PD-L1 binding antagonist inhibits the binding of PD-L1 to its binding partners. In a specific aspect, the PD-L1 binding antagonist inhibits the binding of PD-L1 to PD-1. In another specific aspect, the PD-L1 binding antagonist inhibits the binding of PD-L1 to PD-1 and/or B7-1. In another specific aspect, the PD-L1 binding antagonist inhibits the binding of PD-L1 to both PD-1 and B7-1.

In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, and/or B7-1. In some embodiments, a PD-L1 binding antagonist reduces the negative co-stimulatory signal mediated by or through cell surface proteins expressed on T lymphocytes mediated signaling through PD-L1 so as render a dysfunctional T-cell less non-dysfunctional. In some embodiments, a PD-L1 binding antagonist is an anti-PD-L1 antibody (αPD-L1). In some embodiments, the PD-L1 antibody is a biosimilar, biobetter, or bioequivalent thereof.

In some embodiments, the anti-PD-L1 antibody is BMS-936559 (MDX-1105), AMP-714, atezolizumab (MPDL3280A), durvalumab (MEDI4736), avelumab, or an antibody comprising a VH region produced by the expression vector with ATCC Accession No. PTA-121183 and having the VL region produced by the expression vector with ATCC Accession No. PTA-121182, or a combination thereof.

In some embodiments, the PD-L1 binding antagonist is selected from the group consisting of YW243.55.S70, BMS-936559 (MDX-1105), AMP-714, atezolizumab (MPDL3280A), durvalumab (MEDI4736), avelumab, and an antibody comprising a VH region produced by the expression vector with ATCC Accession No. PTA-121183 and having the VL region produced by the expression vector with ATCC Accession No. PTA-121182.

Some exemplary PD-L1 binding antagonists include those described in U.S. Patent Application Publication 20090055944, U.S. Patent Application Publication 20100203056, U.S. Patent Application Publication 20120039906, U.S. Patent Application Publication 20130045202, U.S. Patent Application Publication 20130309250, U.S. Patent Application Publication US20130034559, U.S. Patent Application Publication US20150282460, U.S. Patent Application Publication 20160108123, PCT Publication No. WO 2011/066389, PCT Publication No. WO 2016/000619, PCT Publication No. WO 2016/094273, PCT Publication No. WO 2016/061142, PCT Publication No. WO 2016/149201, PCT Publication No. WO 2016/149350, PCT Publication No. WO 2016/179576, PCT Publication No. WO 2017/020801, PCT Publication No. WO 2017/103147, PCT Publication No. WO 2017/112741, PCT Publication No. WO 2017/205213, PCT Publication No. WO 2017/054646, PCT Publication No. WO 2017/084495, PCT Publication No. WO 2017/161976, PCT Publication No. WO 2018/005682, PCT Publication No. WO 2018/053106, PCT Publication No. WO 2018/085469, PCT Publication No. WO 2018/111890, and PCT Publication No. WO 2018/106529, the entire disclosures of which are incorporated herein by reference. Other exemplary PD-L1 binding antagonists are described in Sunshine et al., 2015.

The term “PD-L2 binding antagonist,” as used herein, refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In some embodiments, a PD-L2 binding antagonist reduces the negative co-stimulatory signal mediated by or through cell surface proteins expressed on T lymphocytes mediated signaling through PD-L2 so as render a dysfunctional T-cell less non-dysfunctional. In some embodiments, a PD-L2 binding antagonist is a PD-L2 immunoadhesin.

In some embodiments the PD-1 axis binding antagonist (e.g., PD-1 binding antagonist, PD-L1 binding antagonist, or PD-L2 binding antagonist) is a small molecule antagonist. In some further embodiments the PD-1 axis binding antagonist (e.g., PD-L1 binding antagonist) is a chemical compound disclosed in PCT Publication No. WO 2015/033299 or PCT Publication No. WO 2015/033301 or a pharmaceutically acceptable salt thereof, for example, a chemical compound selected from Compound 1 to 25 in Table 2, or a pharmaceutically acceptable salt thereof.

TABLE 2

| Compound | |
|---|---|
| Number | Compound Structure |
| 1 | 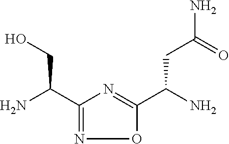 [SMILES: NC(=O)C[C@H](N)c1nc([C@@H](N)CO)no1] |
| 2 | 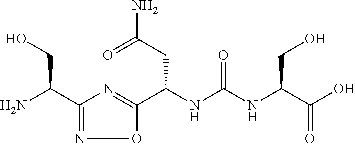 [SMILES: NC(=O)C[C@H](NC(=O)N[C@@H](CO)C(=O)O)c1nc([C@@H](N)CO)no1] |
| 3 | 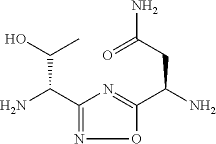 [SMILES: CC(O)[C@@H](N)c1noc([C@H](N)CC(N)=O)n1] |
| 4 | 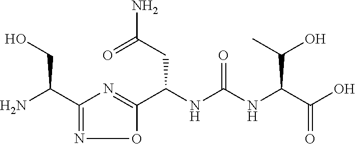 [SMILES: CC(O)[C@H](NC(=O)N[C@@H](CC(N)=O)c1nc([C@@H](N)CO)no1)C(=O)O] |
| 5 | 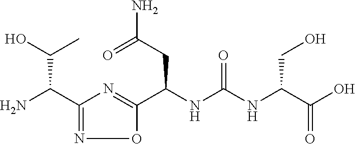 [SMILES: CC(O)[C@@H](N)c1noc([C@@H](CC(N)=O)NC(=O)N[C@H](CO)C(=O)O)n1] |
| 6 | 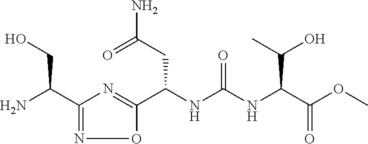 [SMILES: COC(=O)[C@@H](NC(=O)N[C@@H](CC(N)=O)c1nc([C@@H](N)CO)no1)C(C)O] |
| 7 | 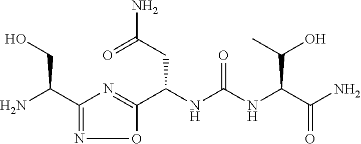 [SMILES: CC(O)[C@H](NC(=O)N[C@@H](CC(N)=O)c1nc([C@@H](N)CO)no1)C(N)=O] |
| 8 | 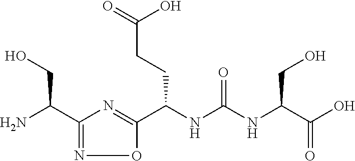 [SMILES: N[C@@H](CO)c1noc([C@H](CCC(=O)O)NC(=O)N[C@@H](CO)C(=O)O)n1] |
| 9 | 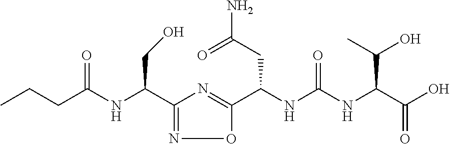 [SMILES: CCCC(=O)N[C@@H](CO)c1noc([C@H](CC(N)=O)NC(=O)N[C@H](C(=O)O)C(C)O)n1] |
| 10 | 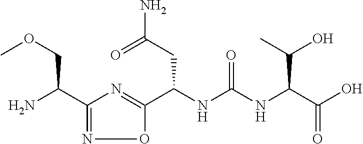 [SMILES: COC[C@H](N)c1noc([C@H](CC(N)=O)NC(=O)N[C@H](C(=O)O)C(C)O)n1] |
| 11 | 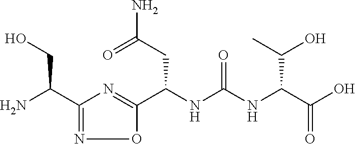 [SMILES: CC(O)[C@@H](NC(=O)N[C@@H](CC(N)=O)c1nc([C@@H](N)CO)no1)C(=O)O] |
| 12 | 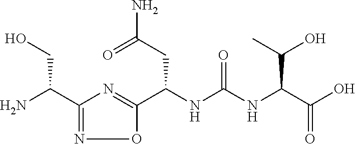 [SMILES: CC(O)[C@H](NC(=O)N[C@@H](CC(N)=O)c1nc([C@H](N)CO)no1)C(=O)O] |
| 13 | 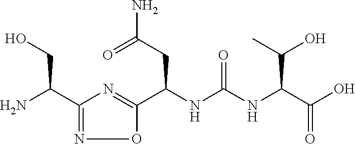 [SMILES: CC(O)[C@H](NC(=O)N[C@H](CC(N)=O)c1nc([C@@H](N)CO)no1)C(=O)O] |
| 14 | 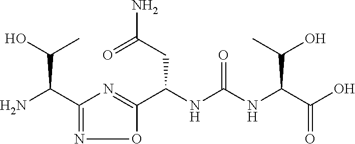 [SMILES: CC(O)[C@H](NC(=O)N[C@@H](CC(N)=O)c1nc([C@@H](N)C(C)O)no1)C(=O)O] |
| 15 | 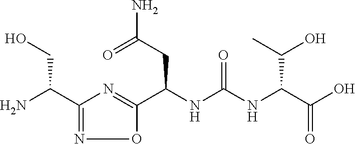 [SMILES: CC(O)[C@@H](NC(=O)N[C@H](CC(N)=O)c1nc([C@H](N)CO)no1)C(=O)O] |
| 16 | 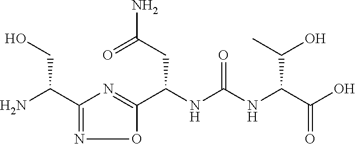 [SMILES: CC(O)[C@@H](NC(=O)N[C@@H](CC(N)=O)c1nc([C@H](N)CO)no1)C(=O)O] |
| 17 | 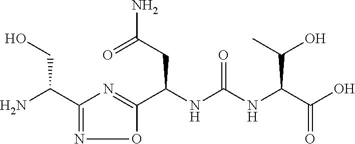 [SMILES: CC(O)[C@H](NC(=O)N[C@H](CC(N)=O)c1nc([C@H](N)CO)no1)C(=O)O] |
| 18 | 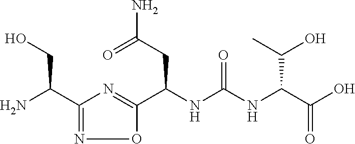 [SMILES: CC(O)[C@@H](NC(=O)N[C@H](CC(N)=O)c1nc([C@@H](N)CO)no1)C(=O)O] |
| 19 | 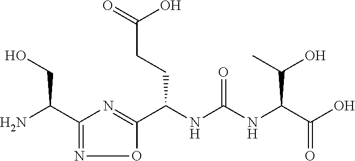 [SMILES: CC(O)[C@H](NC(=O)N[C@@H](CCC(=O)O)c1nc([C@@H](N)CO)no1)C(=O)O] |
| 20 | 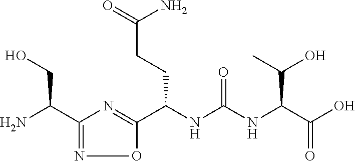 [SMILES: CC(O)[C@H](NC(=O)N[C@@H](CCC(N)=O)c1nc([C@@H](N)CO)no1)C(=O)O] |
| 21 | 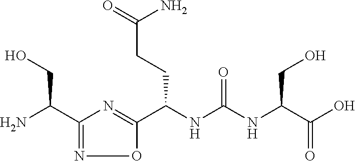 [SMILES: NC(=O)CC[C@H](NC(=O)N[C@@H](CO)C(=O)O)c1nc([C@@H](N)CO)no1] |
| 22 | 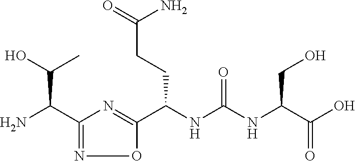 [SMILES: CC(O)[C@H](N)c1noc([C@H](CCC(N)=O)NC(=O)N[C@@H](CO)C(=O)O)n1] |
| 23 | 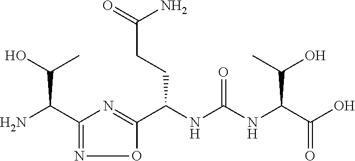 [SMILES: CC(O)[C@H](NC(=O)N[C@@H](CCC(N)=O)c1nc([C@@H](N)C(C)O)no1)C(=O)O] |
| 24 | 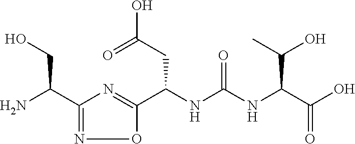 [SMILES: CC(O)[C@H](NC(=O)N[C@@H](CC(=O)O)c1nc([C@@H](N)CO)no1)C(=O)O] |
| 25 | 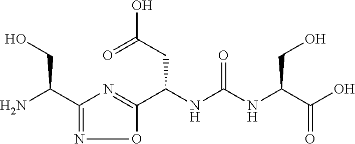 [SMILES: N[C@@H](CO)c1noc([C@H](CC(=O)O)NC(=O)N[C@@H](CO)C(=O)O)n1] |

In some further embodiments the PD-1 axis binding antagonist (e.g., PD-L1 binding antagonist) is Compound Number 12 in Table 2, i.e., (((S)-3-amino-1-(3-((S)-1-amino-2-hydroxyethyl)-1,2,4-oxadiazol-5-yl)-3-oxopropyl)carbamoyl)-L-allothreonine of formula:

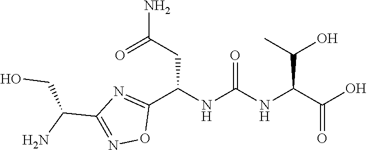 [SMILES: CC(O)[C@H](NC(=O)N[C@@H](CC(N)=O)c1nc([C@H](N)CO)no1)C(=O)O]

or a pharmaceutically acceptable salt thereof.

In some embodiments, the PD-1 axis binding antagonist (e.g., PD-L1 binding antagonist) is 2-(3-(3-amino-1-(3-(1-amino-2-hydroxyethyl)-1,2,4-oxadiazol-5-yl)-3-oxopropyl)ureido)-3-hydroxybutanoic acid:

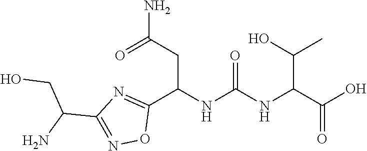 [SMILES: CC(O)C(NC(=O)NC(CC(N)=O)c1nc(C(N)CO)no1)C(=O)O]

or a diastereoismer thereof, or a mixture of diastereoismers thereof, or a pharmaceutically acceptable salt of any of the foregoing.

Table 3 below provides a list of the amino acid sequences of exemplary PD-1 axis binding antagonists for use in the treatment method, medicaments and uses of the present invention. CDRs are underlined for mAb7 and mAb15. The mAB7 is also known as RN888 or PF-6801591. mAb7 (aka RN888) and mAb15 are disclosed in PCT Publication No. WO 2016/092419, the disclosure of which is hereby incorporated by reference in its entirety.

TABLE 3

| mAb7(aka RN 888) | QVQLVQSGAEVKKPGASVKVSCKASGYTFTSYWINWVRQAPG |
|---|---|
| or mAb15 full- | QGLEWMGNIYPGSSLTNYNEKFKNRVTMTRDTSTSTVYMELSS |
| length heavy chain | LRSEDTAVYYCARLSTGTFAYWGQGTLVTVSSASTKGPSVFPL |
| from PCT | APCSRSTSESTAALGCLVKDYFPEPVTVSWNSGALTSGVHTFP |
| Publication No. WO | AVLQSSGLYSLSSVVTVPSSSLGTKTYTCNVDHKPSNTKVDKR |
| 2016/092419 | VESKYGPPCPPCPAPEFLGGPSVFLFPPKPKDTLMISRTPEVTC |
| published on 16- | VVVDVSQEDPEVQFNWYVDGVEVHNAKTKPREEQFNSTYRVV |
| June-2016 | SVLTVLHQDWLNGKEYKCKVSNKGLPSSIEKTISKAKGQPREP |
| (International patent | QVYTLPPSQEEMTKNQVSLTCLVKGFYPSDIAVEWESNGQPEN |
| application number | NYKTTPPVLDSDGSFFLYSRLTVDKSRWQEGNVFSCSVMHEAL |
| PCT/IB2015/059268 | HNHYTQKSLSLSLGK (SEQ ID NO: 1) |
| filed 02- | |
| December-2015) | |
| mAb7 or mAb 15 | QVQLVQSGAEVKKPGASVKVSCKASGYTFTSYWINWVRQAPG |
| full-length heavy | QGLEWMGNIYPGSSLTNYNEKFKNRVTMTRDTSTSTVYMELSS |
| chain without the C- | LRSEDTAVYYCARLSTGTFAYWGQGTLVTVSSASTKGPSVFPL |
| terminal lysine | APCSRSTSESTAALGCLVKDYFPEPVTVSWNSGALTSGVHTFP |
| from PCT | AVLQSSGLYSLSSVVTVPSSSLGTKTYTCNVDHKPSNTKVDKR |
| Publication No. WO | VESKYGPPCPPCPAPEFLGGPSVFLFPPKPKDTLMISRTPEVTC |
| 2016/092419 | VVVDVSQEDPEVQFNWYVDGVEVHNAKTKPREEQFNSTYRVV |
| | SVLTVLHQDWLNGKEYKCKVSNKGLPSSIEKTISKAKGQPREP |
| | QVYTLPPSQEEMTKNQVSLTCLVKGFYPSDIAVEWESNGQPEN |
| | NYKTTPPVLDSDGSFFLYSRLTVDKSRWQEGNVFSCSVMHEAL |
| | HNHYTQKSLSLSLG (SEQ ID NO: 2) |
| mAb7 full-length | DIVMTQSPDSLAVSLGERATINCKSSQSLWDSGNQKNFLTWYQ |
| light chain | QKPGQPPKLLIYWTSYRESGVPDRFSGSGSGTDFTLTISSLQAE |
| from PCT | DVAVYYCQNDYFYPHTFGGGTKVEIKRGTVAAPSVFIFPPSDE |
| Publication No. WO | QLKSGTASVVCLLNNFYPREAKVQWKVDNALQSGNSQESVTE |
| 2016/092419 | QDSKDSTYSLSSTLTLSKADYEKHKVYACEVTHQGLSSPVTKS |
| | FNRGEC (SEQ ID NO: 3) |
| mAb7 light chain | QVQLVQSGAEVKKPGASVKVSCKASGYTFTSYWINWVRQAPG |
| variable region | QGLEWMGNIYPGSSLTNYNEKFKNRVTMTRDTSTSTVYMELSS |
| WO 2016/092419 | LRSEDTAVYYCARLSTGTFAYWGQGTLVTVSS (SEQ ID NO: 4) |
| mAB7 and mAB15 | QVQLVQSGAEVKKPGASVKVSCKASGYTFTSYWINWVRQAPG |
| heavy chain | QGLEWMGNIWPGSSLTNYNEKFKNRVTMTRDTSTSTVYMELS |
| variable region | SLRSEDTAVYYCARLLTGTFAYWGQGTLVTVSS (SEQ ID NO: 5) |
| from PCT | |
| Publication No. WO | |
| 2016/092419 | |
| mAb15 light chain | DIVMTQSPDSLAVSLGERATINCKSSQSLWDSGNQKNFLTWYQ |
| variable region | QKPGQPPKLLIYWTSYRESGVPDRFSGSGSGTDFTLTISSLQAE |
| WO 2016/092419 | DVAVYYCQNDYFYPHTFGGGTKVEIK (SEQ ID NO: 6) |
| Nivolumab, | QVQLVESGGGWQPGRSLRLDCKASGITFSNSGMHWVRQAPG |
| MDX1106, full | KGLEWVAVrWYDGSKRYYADSVKGRFTISRDNSKNTLFLQMNS |
| length heavy chain | LRAEDTAVYYCATNDDYWGQGTLVTVSSASTKGPSVFPLAPCS |
| from PCT | RSTSESTAALGCLVDYFPEPVTVSWNSGALTSGVHTFPAVLQS |
| Publication No. WO | SGLYSLSSVVTVPSSSLGTTYTCNVDHKPSNTKVDRVESYGPP |
| 2006/121168 | CPPCPAPEFLGGPSVFLFPPKPKDTLMISRTPEVTCVVVDVSQE |
| | DPEVQFNWYYDGVEVHNATKPREEQFNSTYRVVSVLTVLHQD |
| | WLNGKEYKCKVSNKGLPSSIEKTISKA |
| | GQPREPQVYTLPPSQEEMTKNQVSLTCLVKGFYPSDIAVEWES |
| | NGQPEKNYKTTPPVLDSDGSFFLYSRLTVDKSRWQEGNVFSC |
| | SVMHEALHNHYTQKSLSLSLGK (SEQ ID NO: 7) |
| Nivolumab, | EIVLTQSPATLSLSPGERATLSCRASQSVSSYLAWYQQPGQAP |
| MDX1106, full | RLLIYDASNRATGIPARFSGSGSGTDFTLTISSLEPEDFAVYYCQ |
| length light chain | QSSNWPRTFGQGTKVEIRTVAAPSVFIFPPSDEQLSGTASVVCL |
| from PCT | LNNFYPREAVQWKVDNALQSGNSQESVTEQDSDSTYSLSSTL |
| Publication No. WO | TLSKADYEKHKVYACEVTHQGLSSPVTSFNRGEC (SEQ ID NO: |
| 2006/121168 | 8) |
| Pembrolizumab, | QVQLVQSGVEVKKPGASVKVSCKASGYTFTNYYMYWVRQA |
| MK3475, full length | PGQGLEWMGGINPSNGGTNFNEKFKNRVTLTTDSSTTTAYM |
| heavy chain | ELKSLQFDDTAVYYCARRDYRFDMGFDYWGQGTTVTVSSA |
| from PCT | STKGPSVFPLAPCSRSTSESTAALGCLVKDYFPEPVTVSWNS |
| Publication No. WO | GALTSGVHTFPAVLQSSGLYSLSSVVTVPSSSLGTKTYTCNV |
| 2009/114335 | DHKPSNTKVDKRVESKYGPPCPPCPAPEFLGGPSVFLFPPK |
| | PKDTLMISRTPEVTCVVVDVSQEDPEVQFNWYVDGVEVHNA |
| | KTKPREEQFNSTYRVVSVLTVLHQDWLNGKEYKCKVSNKGL |
| | PSSIEKTISKAKGQPREPQVYTLPPSQEEMTKNQVSLTCLVK |
| | GFYPSDIAVEWESNGQPENNYKTTPPVLDSDGSFFLYSRLTV |
| | DKSRWQEGNVFSCSVMHEALHNHYTQKSLSLSLGK (SEQ ID |
| | NO: 9) |
| Pembrolizumab, | EIVLTQSPATLSLSPGERATLSCRASKGVSTSGYSYLHWYQQ |
| MK3475, full length | KPGQAPRLLIYLASYLESGVPARFSGSGSGTDFTLTISSLEPE |
| light chain | DFAVYYCQHSRDLPLTFGGGTKVEIKRTVAAPSVFIFPPSDE |
| from PCT | QLKSGTASVVCLLNNFYPREAKVQWKVDNALQSGNSQESVT |
| Publication No. WO | EQDSKDSTYSLSSTLTLSKADYEKHKVYACEVTHQGLSSPVT |
| 2009/114335 | KSFNRGEC (SEQ ID NO: 10) |
| AMP-224 (or | LFTVTVPKELYIIEHGSNVTLECNFDTGSHVNLGAITASLQKVEN |
| AMP224), without | DTSPHRERATLLEEQLPLGKASFHIPQVQVRDEGQYQCIIIYGVA |
| signal sequence | WDYKYLTLKVKASYRKINTHILKVPETDEVELTCQATGYPLAEV |
| from PCT | SWPNVSVPANTSHSRTPEGLYQVTSVLRLKPPPGRNFSCVFW |
| Publication No. WO | NTHVRELTLASIDLQSQMEPRTHPTWEPKSCDKTHTCPPCPAP |
| 2010/027827 and | ELLGGPSVFLFPPKPKDTLMISRTPEVTCVVVDVSHEDPEVKFN |
| PCT Publication | WYVDGVEVHNAKTKPREEQYNSTYRWSVLTVLHQDWLNGKE |
| No. WO | YKCKVSNKALPAPIEKTISKAKGQPREPQVYTLPPSRDELTKNQ |
| 2011/066342 | VSLTCLVKGFYPSDIAVEWESNGQPENNYKTTPPVLDSDGS |
| | FFLYSKLTVDKSRWQQGNVFSCSVMHEALHNHYTQKSLSLSP |
| | GK (SEQ ID NO: 11) |
| YW243.55.S70 | EVQLVESGGGLVQPGGSLRLSCAASGFTFSDSWIHWVRQAPG |
| heavy chain | KGLEWVAWISPYGGSTYYADSVKGRFTISADTSKNTAYLQMNS |
| variable region | LRAEDTAVYYCARRHWPGGFDYWGQGTLVTVSA (SEQ ID NO: |
| from PCT | 12) |
| Publication No. WO | |
| 2010/077634 | |
| YW243.55.S70 light | DIQMTQSPSSLSASVGDRVTITCRASQDVSTAVAWYQQKPGKA |
| chain variable | PKLLIYSASFLYSGVPSRFSGSGSGTDFTLTISSLQPEDFATYYC |
| region | QQYLYHPATFGQGTKVEIKR (SEQ ID NO: 13) |
| from PCT | |
| Publication No. WO | |
| 2010/077634 | |
| avelumab heavy | EVQLLESGGGLVQPGGSLRLSCAASGFTFSSYIMWVRQAPGK |
| chain variable | GLEWVSSIYPSGGITFYADKGRFTISRDNSKNTLYLQMNSLRAE |
| region | DTAVYYCARIKLGTVTTVDYWGQGTLVTVSS (SEQ ID NO: 14) |
| from PCT | |
| Publication No. WO | |
| 13079174 | |
| avelumab light | QSALTQPASVSGSPGQSITISCTGTSSDVGGYNYVSWYQQHP |
| chain variable | GKAPKLMIYDVSNRPSGVSNRFSGSKSGNTASLTISGLQAEDE |
| region | ADYYCSSYTSSSTRVFGTGTKVTVL (SEQ ID NO: 15) |
| from PCT | |
| Publication No. WO | |
| 2013/079174 | |
| AGEN-2034w | QVQLVESGGGVVQPGRSLRLSCAASGFTFSSYGMHWVRQAP |
| Full length heavy | GKGLEWVAVIWYDGSNKYYADSVKGRFTISRDNSKNTLYLQMN |
| chain | SLRAEDTAVYYCASNGDHWGQGTLVTVSSASTKGPSVFPLAP |
| CAS RN: 2088287- | CSRSTSESTAALGCLVKDYFPEPVTVSWNSGALTSGVHTFPAV |
| 86-7 | LQSSGLYSLSSVVTVPSSSLGTKTYTCNVDHKPSNTKVDKRVE |
| from PCT | SKYGPPCPPCPAPEFLGGPSVFLFPPKPKDTLMISRTPEVTCVV |
| Publication No. WO | VDVSQEDPEVQFNWYVDGVEVHNAKTKPREEQFNSTYRVVSV |
| 2017/040790 | LTVLHQDWLNGKEYKCKVSNKGLPSSIEKTISKAKGQPREPQV |
| | YTLPPSQEEMTKNQVSLTCLVKGFYPSDIAVEWESNGQPENNY |
| | KTTPPVLDSDGSFFLYSRLTVDKSRWQEGNVFSCSVMHEALH |
| | NHYTQKSLSLSLG (SEQ ID NO: 16) |
| AGEN2034w | EIVMTQSPATLSVSPGERATLSCRASQSVSSNLAWYQQKPGQ |
| full length light | APRLLIYGASTRATGIPARFSGSGSGTEFTLTISSLQSEDFAVYY |
| chain | CQQYNNWPRTFGQGTKVEIKRTVAAPSVFIFPPSDEQLKSGTA |
| CAS RN: 2088287- | SVVCLLNNFYPREAKVQWKVDNALQSGNSQESVTEQDSKDST |
| 75-4 | YSLSSTLTLSKADYEKHKVYACEVTHQGLSSPVTKSFNRGEC |
| from PCT | (SEQ ID NO: 17) |
| Publication No. WO | |
| 2017/040790 | |
| Spartalizumab | EVQLVQSGAEVKKPGESLRISCKGSGYTFTTYWMHWVRQATG |
| Full length heavy | QGLEWMGNIYPGTGGSNFDEKFKNRVTITADKSTSTAYMELSS |
| chain | LRSEDTAVYYCTRWTTGTGAYWGQGTTVWSSASTKGPSVFP |
| CAS RN: 1935694- | LAPCSRSTSESTAALGCLVKDYFPEPVTVSWNSGALTSGVHTF |
| 88-4 | PAVLQSSGLYSLSSVVTVPSSSLGTKTYTCNVDHKPSNTKVDK |
| from PCT | RVESKYGPPCPPCPAPEFLGGPSVFLFPPKPKDTLMISRTPEVT |
| Publication | CVVVDVSQEDPEVQFNWYVDGVEVHNAKTKPREEQFNSTYRV |
| No.WO/2015/112900 | VSVLTVLHQDWLNGKEYKCKVSNKGLPSSIEKTISKAKGQPRE |
| | PQVYTLPPSQEEMTKNQVSLTCLVKGFYPSDIAVEWESNGQPE |
| | NNYKTTPPVLDSDGSFFLYSRLTVDKSRWQEGNVFSCSVMHE |
| | ALHNHYTQKSLSLSLG (SEQ ID NO: 18) |
| Spartalizumab Full | EIVLTQSPATLSLSPGERATLSCKSSQSLLDSGNQKNFLTWYQ |
| length light chain | QKPGQAPRLLIYWASTRESGVPSRFSGSGSGTDFTFTISSLEAE |
| CAS RN: 1935694- | DAATYYCQNDYSYPYTFGQGTKVEIKRTVAAPSVFIFPPSDEQL |
| 88-4 | KSGTASVVCLLNNFYPREAKVQWKVDNALQSGNSQESVTEQD |
| from PCT | SKDSTYSLSSTLTLSKADYEKHKVYACEVTHQGLSSPVTKSFN |
| Publication No. WO | RGEC (SEQ ID NO: 19) |
| 2015/112900 | |
| Cemiplimab | EVQLLESGGVLVQPGGSLRLSCAASGFTFSNFGMTWVRQAPG |
| (REGN-2810) | KGLEWVSGISGGGRDTYFADSVKGRFTISRDNSKNTLYLQMNS |
| CAS RN: 1801342- | LKGEDTAVYYCVKWGNIYFDYWGQGTLVTVSSASTKGPSVFPL |
| 60-8 | APCSRSTSESTAALGCLVKDYFPEPVTVSWNSGALTSGVHTFP |
| Full length heavy | AVLQSSGLYSLSSVVTVPSSSLGTKTYTCNVDHKPSNTKVDKR |
| chain | VESKYGPPCPPCPAPEFLGGPSVFLFPPKPKDTLMISRTPEVTC |
| from PCT | VVVDVSQEDPEVQFNWYVDGVEVHNAKTKPREEQFNSTYRVV |
| Publication No. WO | SVLTVLHQDWLNGKEYKCKVSNKGLPSSIEKTISKAKGQPREP |
| 2015/112800 | QVYTLPPSQEEMTKNQVSLTCLVKGFYPSDIAVEWESNGQPEN |
| | NYKTTPPVLDSDGSFFLYSRLTVDKSRWQEGNVFSCSVMHEAL |
| | HNHYTQKSLSLSLGK (SEQ ID NO: 20) |
| Cemiplimab | DIQMTQSPSSLSASVGDSITITCRASLSINTFLNWYQQKPGKAP |
| (REGN-2810) | NLLIYAASSLHGGVPSRFSGSGSGTDFTLTIRTLQPEDFATYYC |
| CAS RN: 1801342- | QQSSNTPFTFGPGTVVDFRRTVAAPSVFIFPPSDEQLKSGTAS |
| 60-8 | VVCLLNNFYPREAKVQWKVDNALQSGNSQESVTEQDSKDSTY |
| full length light | SLSSTLTLSKADYEKHKVYACEVTHQGLSSPVTKSFNRGEC |
| chain | (SEQ ID NO: 21) |
| from PCT | |
| Publication No. WO | |
| 2015/112800 | |
| Durvalumab (MEDI- | EVQLVESGGGLVQPGGSLRLSCAASGFTFSRYWMSWVRQAP |
| 4736 or IMFINZI ®) | GKGLEWVANIKQDGSEKYYVDSVKGRFTISRDNAKNSLYLQMN |
| CAS RN: 2222916- | SLRAEDTAVYYCAREGGWFGELAFDYWGQGTLVTVSSASTKG |
| 00-7 | PSVFPLAPSSKSTSGGTAALGCLVKDYFPEPVTVSWNSGALTS |
| Full length heavy | GVHTFPAVLQSSGLYSLSSVVTVPSSSLGTQTYICNVNHKPSNT |
| chain | KVDKRVEPKSCDKTHTCPPCPAPEFEGGPSVFLFPPKPKDTLM |
| from PCT | ISRTPEVTCVVVDVSHEDPEVKFNWYVDGVEVHNAKTKPREEQ |
| Publication No. WO | YNSTYRVVSVLTVLHQDWLNGKEYKCKVSNKALPASIEKTISKA |
| 2011/066389 and. | KGQPREPQVYTLPPSREEMTKNQVSLTCLVKGFYPSDIAVEWE |
| WO 2018/106529 | SNGQPENNYKTTPPVLDSDGSFFLYSKLTVDKSRWQQGNVFS |
| | CSVMHEALHNHYTQKSLSLSPGK (SEQ ID NO: 22) |
| Durvalumab (MEDI- | EIVLTQSPGTLSLSPGERATLSCRASQRVSSSYLAWYQQKPGQ |
| 4736 or IMFINZI ®) | APRLLIYDASSRATGIPDRFSGSGSGTDFTLTISRLEPEDFAVYY |
| CAS RN: 2222915- | CQQYGSLPWTFGQGTKVEIKRTVAAPSVFIFPPSDEQLKSGTA |
| 99-1 | SVVCLLNNFYPREAKVQWKVDNALQSGNSQESVTEQDSKDST |
| full length light | YSLSSTLTLSKADYEKHKVYACEVTHQGLSSPVTKSFNRGEC |
| chain | (SEQ ID NO: 23) |
| from PCT | |
| Publication No. WO | |
| 2011/066389 and | |
| WO 2018/106529 | |
| Atezolizumab | EVQLVESGGGLVQPGGSLRLSCAASGFTFSDSWIHWVRQAPG |
| (MPDL3280A or | KGLEWVAWISPYGGSTYYADSVKGRFTISADTSKNTAYLQMNS |
| RG7446) | LRAEDTAVYYCARRHWPGGFDYWGQGTLVTVSSASTKGPSVF |
| CAS number: | PLAPSSKSTSGGTAALGCLVKDYFPEPVTVSWNSGALTSGVHT |
| 1380723-44-3 | FPAVLQSSGLYSLSSVVTVPSSSLGTQTYICNVNHKPSNTKVDK |
| Full length heavy | KVEPKSCDKTHTCPPCPAPELLGGPSVFLFPPKPKDTLMISRTP |
| chain | EVTCVVVDVSHEDPEVKFNWYVDGVEVHNAKTKPREEQYAST |
| From PCT | YRVVSVLTVLHQDWLNGKEYKCKVSNKALPAPIEKTISKAKGQP |
| Publication No. WO | REPQVYTLPPSREEMTKNQVSLTCLVKGFYPSDIAVEWESNGQ |
| 2018/106529 | PENNYKTTPPVLDSDGSFFLYSKLTVDKSRWQQGNVFSCSVM |
| | HEALHNHYTQKSLSLSPGK (SEQ ID NO: 24) |
| Atezolizumab | DIQMTQSPSSLSASVGDRVTITCRASQDVSTAVAWYQQKPGKA |
| full length light | PKLLIYSASFLYSGVPSRFSGSGSGTDFTLTISSLQPEDFATYYC |
| chain | QQYLYHPATFGQGTKVEIKRTVAAPSVFIFPPSDEQLKSGTASV |
| from PCT | VCLLNNFYPREAKVQWKVDNALQSGNSQESVTEQDSKDSTYS |
| Publication No. WO | LSSTLTLSKADYEKHKVYACEVTHQGLSSPVTKSFNRGEC |
| 2018 106529 | (SEQ ID NO: 25) |
| KN-035 (or KN035) | QVQLVESGGGLVQPGGSLRLSCAASGFTFSRRCMAWFRQAP |
| Single domain | GKERERVAKLLTTSGSTYLADSVKGRFTISRDNSKNTVYLQMN |
| antibody | SLRAEDTAVYYCAADSFEDPTCTLVTSSGAFQYWGQGTLVTVS |
| From European | S (SEQ ID NO: 26) |
| Publication No. | |
| EP3330290A1, | |
| (aka, hu56V2) | |
| MDX-1105 (BMS- | QVQLVQSGAEVKKPGSSVKVSCKTSGDTFSTYAISWVRQAPG |
| 936559) | QGLEWMGGIIPIFGKAHYAQKFQGRVTITADESTSTAYMELSSL |
| Heavy chain | RSEDTAVYFCARKFHFVSGSPFGMDVWGQGTTVTVSS (SEQ |
| variable region | ID NO: 27) |
| From PCT | |
| Publication No. WO | |
| 2018/106529 and | |
| WO 2007/005874 | |
| MDX-1105 (BMS- | EIVLTQSPATLSLSPGERATLSCRASQSVSSYLAWYQQKPGQA |
| 936559) | PRLLIYDASNRATGIPARFSGSGSGTDFTLTISSLEPEDFAVYYC |
| Light chain variable | QQRSNWPTFGQGTKVEIK (SEQ ID NO: 28) |
| region | |
| from PCT | |
| Publication No. WO | |
| 2018/106529 and | |
| WO 2007/005874 | |
| CT-011 | QVQLVQSGSELKKPGASVKISCKASGYTFTNYGMNWVRQAPG |
| (pidilizumab), | QGLQWMGWINTDSGESTYAEEFKGRFVFSLDTSVNTAYLQITS |
| Full length heavy | LTAEDTGMYFCVRVGYDALDYWGQGTLVTVSSASTKGPSVFP |
| chain | LAPSSKSTSGGTAALGCLVKDYFPEPVTVSWNSGALTSGVHTF |
| from PCT | PAVLQSSGLYSLSSVVTVPSSSLGTQTYICNVNHKPSNTKVDKR |
| Publication No. WO | VEPKSCDKTHTCPPCPAPELLGGPSVFLFPPKPKDTLMISRTPE |
| 2009/101611 | VTCVVVDVSHEDPEVKFNWYVDGVEVHNAKTKPREEQYNSTY |
| | RVVSVLTVLHQDWLNGKEYKCKVSNKALPAIEKTISKAKGQPR |
| | EPQVYTLPPSREEMTKNQVSLTCLVKGFYPSDIAVEWESNGQP |
| | ENNYKTTPPVLDSDGSFFLYSKLTVDKSRWQQGNVFSCSVMH |
| | EALHNHYTQKSLSLSPGK (SEQ ID NO: 29) |
| CT-011 | EIVLTQSPSSLSASVGDRVTITCSARSSVSYMHWFQQKPGKAP |
| (pidilizumab), | KLWIYRTSNLASGVPSRFSGSGSGTSYCLTINSLQPEDFATYYC |
| Full length light | QQRSSFPLTFGGGTKLEIKRTVAAPSVFIFPPSDEQLKSGTASV |
| chain | VCLLNNFYPREAKVQWKVDNALQSGNSQESVTEQDSKDSTYS |
| from PCT | LSSTLTLSKADYEKHKVYACEVTHQGLSSPVTKSFNRGEC |
| Publication No. WO | (SEQ ID NO: 30) |
| 2009/101611 | |
| BGB-A317 | QVQLQESGPGLVKPSETLSLTCTVSGFSLTSYGVHWIRQPPGK |
| tislelizumab, (BGB- | GLEWIGVIYADGSTNYNPSLKSRVTISKDTSKNQVSLKLSSVTA |
| 108, BGB-A317) | ADTAVYYCARAYGNYWYIDVWGQGTTVTVSSASTKGPSVFPL |
| CAS Registry | APCSRSTSESTAALGCLVKDYFPEPVTVSWNSGALTSGVHTFP |
| Number: 1858168- | AVLQSSGLYSLSSVVTVPSSSLGTKTYTCNVDHKPSNTKVDKR |
| 59-8 | VESKYGPPCPPCPAPPVAGGPSVFLFPPKPKDTLMISRTPEVT |
| | CVVVAVSQEDPEVQFNWYVDGVEVHNAKTKPREEQFNSTYRV |
| Full length heavy | VSVLTVVHQDWLNGKEYKCKVSNKGLPSSIEKTISKAKGQPRE |
| chain | PQVYTLPPSQEEMTKNQVSLTCLVKGFYPSDIAVEWESNGQPE |
| from PCT | NNYKTTPPVLDSDGSFFLYSKLTVDKSRWQEGNVFSCSVMHE |
| Publication No. WO | ALHNHYTQKSLSLSLGK (SEQ ID NO: 31) |
| 2015/035606 | |
| BGB-A317 | DIVMTQSPDSLAVSLGERATINCKSSESVSNDVAWYQQKPGQP |
| tislelizumab, (BGB- | PKLLINYAFHRFTGVPDRFSGSGYGTDFTLTISSLQAEDVAVYY |
| 108, BGB-A317 | CHQAYSSPYTFGQGTKLEIKRTVAAPSVFIFPPSDEQLKSGTAS |
| CAS Registry | VVCLLNNFYPREAKVQWKVDNALQSGNSQESVTEQDSKDSTY |
| Number: 1858168- | SLSSTLTLSKADYEKHKVYACEVTHQGLSSPVTKSFNRGEC |
| 59-8 | (SEQ ID NO: 32) |
| Full length light | |
| chain | |
| from PCT | |
| Publication No. WO | |
| 2015/035606 | |

As used herein, an anti-human PD-L1 mAb refers to a monoclonal antibody that specifically binds to mature human PD-L1. A mature human PD-L1 molecule consists of amino acids 19-290 of the following sequence

(SEQ ID NO: 33)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET.

Table 4 below provides exemplary anti-PD-L1 antibody sequences for use in the treatment method, medicaments and uses of the present invention.

TABLE 4
EXEMPLARY ANTI-HUMAN PD-L1 MONOCLONAL ANTIBODY SEQUENCES

| | |
|---|---|
| Heavy chain | SYIMM (SEQ ID NO: 34) |
| CDR1 (CDRH1) | |
| Heavy chain | SIYPSGGITFY (SEQ ID NO: 35) |
| CDR2 (CDRH2) | |
| Heavy chain | IKLGTVTTVDY (SEQ ID NO: 36) |
| CDR3 (CDRH3) | |
| Light chain | TGTSSDVGGYNYVS (SEQ ID NO: 37) |
| CDR1 (CDRL1) | |
| Light chain | DVSNRPS (SEQ ID NO: 38) |
| CDR2 (CDRL2) | |
| Light chain | SSYTSSSTRV (SEQ ID NO: 39) |
| CDR3 (CDRL3) | |
| Heavy chain | EVQLLESGGGLVQPGGSLRLSCAASGFTFSSYIMMWVR |
| variable region | QAPGKGLEWVSSIYPSGGITFYADKGRFTISRDNSKNTL |
| (VR) | YLQMNSLRAEDTAVYYCARIKLGTVTTVDYWGQGTLVT |
| | VSS (SEQ ID NO: 14) |
| Light chain VR | QSALTQPASVSGSPGQSITISCTGTSSDVGGYNYVSWY |
| | QQHPGKAPKLMIYDVSNRPSGVSNRFSGSKSGNTASLTI |
| | SGLQAEDEADYYCSSYTSSSTRVFGTGTKVTVL (SEQ ID |
| | NO: 15) |
| Heavy chain | EVQLLESGGGLVQPGGSLRLSCAASGFTFSSYIMMWVR |
| | QAPGKGLEWVSSIYPSGGITFYADTVKGRFTISRDNSKN |
| | TLYLQMNSLRAEDTAVYYCARIKLGTVTTVDYWGQGTLV |
| | TVSSASTKGPSVFPLAPSSKSTSGGTAALGCLVKDYFPE |
| | PVTVSWNSGALTSGVHTFPAVLQSSGLYSLSSVVTVPS |
| | SSLGTQTYICNVNHKPSNTKVDKKVEPKSCDKTHTCPPC |
| | PAPELLGGPSVFLFPPKPKDTLMISRTPEVTCVVVDVSH |
| | EDPEVKFNWYVDGVEVHNAKTKPREEQYNSTYRVVSVL |
| | TVLHQDWLNGKEYKCKVSNKALPAPIEKTISKAKGQPRE |
| | PQVYTLPPSRDELTKNQVSLTCLVKGFYPSDIAVEWESN |
| | GQPENNYKTTPPVLDSDGSFFLYSKLTVDKSRWQQGNV |
| | FSCSVMHEALHNHYTQKSLSLSPGK (SEQ ID NO: 40) |
| Light chain | QSALTQPASVSGSPGQSITISCTGTSSDVGGYNYVSWY |
| | QQHPGKAPKLMIYDVSNRPSGVSNRFSGSKSGNTASLTI |
| | SGLQAEDEADYYCSSYTSSSTRVFGTGTKVTVLGQPKA |
| | NPTVTLFPPSSEELQANKATLVCLISDFYPGAVTVAWKA |
| | DGSPVKAGVETTKPSKQSNNKYAASSYLSLTPEQWKSH |
| | RSYSCQVTHEGSTVEKTVAPTECS (SEQ ID NO: 41) |

In some embodiments, the PD-1 axis binding antagonist is avelumab and will be administered intravenously ata dose of about 1, 2, 3, 4, 5, 6, 7, 8, 9, 10, 11, 12, 13, 14, 15, 16, 17, 18, 19 or 20 mg/kg at intervals of about 14 days (±2 days) or about 21 days (±2 days) or about 30 days (±2 days) throughout the course of treatment. In some embodiment, avelumab is administered as a flat dose of about 80, 150, 160, 200, 240, 250, 300, 320, 350, 400, 450, 480, 500, 550, 560, 600, 640, 650, 700, 720, 750, 800, 850, 880, 900, 950, 960, 1000, 1040, 1050, 1100, 1120, 1150, 1200, 1250, 1280, 1300, 1350, 1360, 1400, 1440, 1500, 1520, 1550 or 1600 mg, preferably 800 mg, 1200 mg or 1600 mg at intervals of about 14 days (±2 days) or about 21 days (±2 days) or about 30 days (±2 days) throughout the course of treatment. In certain embodiments, a subject will be administered an intravenous (IV) infusion of a medicament comprising any of the PD-1 axis binding antagonists described herein. In certain embodiment, the subject will be administered a subcutaneous (SC) infusion of a medicament comprising any of the PD-1 axis binding antagonist described herein.

In some embodiments, the PD-1 axis binding antagonist is RN888 and will be administered subcutaneously at a dose of about 1, 2, 3, 4, 5, 6, 7 or 8 mg/kg at intervals of about 14 days (±2 days) or about 21 days (±2 days) or about 30 days (±2 days) throughout the course of treatment. In some embodiment, RN888 is administer as a flat dose of about 80, 150, 160, 200, 240, 250, 300, 320, 350, 400, preferably 300mg at intervals of about 14 days (±2 days) or about 21 days (±2 days) or about 30 days (±2 days). In some embodiments, RN888 is administered subcutaneously in an amount of 300 mg Q4W.

In one embodiment, “PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment method, medicaments and uses of the present invention in which a human individual is being treated, the PD-1 antagonist may block binding of human PD-L1 to human PD-1, and block binding of both human PD-L1 and PD-L2 to human PD-1. Exemplary human PD-1 amino acid sequences can be found in NCBI Locus No.: NP_005009. Exemplary human PD-L1 and PD-L2 amino acid sequences can be found in NCBI Locus No.: NP_054862 and NP_079515, respectively.

PD-1 antagonists useful in any of the treatment methods, medicaments and uses of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in some embodiments, the human constant region is an IgG1 or IgG4 constant region. In some embodiments, the antigen binding fragment is selected from the group consisting of Fab, Fab′-SH, F(ab′)₂, scFv and Fv fragments.

Examples of mAbs that bind to human PD-1, and useful in the treatment method, medicaments and uses of the present invention, are described in U.S. Pat. Nos. 7,488,802, 7,521,051, 8,008,449, 8,354,509, 8,168,757, PCT Patent Publication Nos. WO 2004/004771, WO 2004/072286, WO 2004/056875, and US Patent Publication No. 2011/0271358. Specific anti-human PD-1 mAbs useful as the PD-1 antagonist in the treatment method, medicaments and uses of the present invention include: nivolumab (MDX 1106), pembrolizumab (MK-3475), pidilizumab (CT-011), cemiplimab (REGN2810), tislelizumab (BGB-A317), spartalizumab (PDR001), RN888, mAb15, MEDI-0680 (AMP-514), BGB-108, or AGEN-2034, or a combination thereof.

Table 5 below provides exemplary anti-PD-1 antibody sequences for use in the treatment method, medicaments and uses of the present invention.

TABLE 5
EXEMPLARY ANTI-HUMAN PD-1 MONOCLONAL ANTIBODY (RN888)
SEQUENCES

| Heavy chain | GYTFTSYWIN (SEQ ID NO: 42) |
|---|---|
| CDR1 (CDRH1) | |
| Heavy chain | NIYPGSSLTNYNEKFKN (SEQ ID NO: 43) |
| CDR2 (CDRH2) | |
| Heavy chain | LSTGTFAY (SEQ ID NO: 44) |
| CDR3 (CDRH3) | |
| Light chain | KSSQSLWDSGNQKNFLT (SEQ ID NO: 45) |
| CDR1 (CDRL1) | |
| Light chain | WTSYRES (SEQ ID NO: 46) |
| CDR2 (CDRL2) | |
| Light chain | QNDYFYPHT (SEQ ID NO: 47) |
| CDR3 (CDRL3) | |
| Heavy chain | QVQLVQSGAEVKKPGASVKVSCKASGYTFTSYWINWVR |
| variable region | QAPGQGLEWMGNIYPGSSLTNYNEKFKNRVTMTRDTST |
| (VR) | STVYMELSSLRSEDTAVYYCARLSTGTFAYWGQGTLVT |
| | VSS (SEQ ID NO: 4) |
| Light chain VR | DIVMTQSPDSLAVSLGERATINCKSSQSLWDSGNQKNFL |
| | TWYQQKPGQPPKLLIYWTSYRESGVPDRFSGSGSGTD |
| | FTLTISSLQAEDVAVYYCQNDYFYPHTFGGGTKVEIK |
| | (SEQ ID NO: 6) |
| Heavy chain | QVQLVQSGAEVKKPGASVKVSCKASGYTFTSYWINWVR |
| | QAPGQGLEWMGNIYPGSSLTNYNEKFKNRVTMTRDTST |
| | STVYMELSSLRSEDTAVYYCARLSTGTFAYWGQGTLVT |
| | VSSASTKGPSVFPLAPCSRSTSESTAALGCLVKDYFPEP |
| | VTVSWNSGALTSGVHTFPAVLQSSGLYSLSSVVTVPSS |
| | SLGTKTYTCNVDHKPSNTKVDKRVESKYGPPCPPCPAP |
| | EFLGGPSVFLFPPKPKDTLMISRTPEVTCVVVDVSQEDP |
| | EVQFNWYVDGVEVHNAKTKPREEQFNSTYRVVSVLTVL |
| | HQDWLNGKEYKCKVSNKGLPSSIEKTISKAKGQPREPQ |
| | VYTLPPSQEEMTKNQVSLTCLVKGFYPSDIAVEWESNG |
| | QPENNYKTTPPVLDSDGSFFLYSRLTVDKSRWQEGNVF |
| | SCSVMHEALHNHYTQKSLSLSLGK (SEQ ID NO: 1) |
| Light chain | DIVMTQSPDSLAVSLGERATINCKSSQSLWDSGNQKNFL |
| | TWYQQKPGQPPKLLIYWTSYRESGVPDRFSGSGSGTD |
| | FTLTISSLQAEDVAVYYCQNDYFYPHTFGGGTKVEIKRG |
| | TVAAPSVFIFPPSDEQLKSGTASVVCLLNNFYPREAKVQ |
| | WKVDNALQSGNSQESVTEQDSKDSTYSLSSTLTLSKAD |
| | YEKHKVYACEVTHQGLSSPVTKSFNRGEC (SEQ ID NO: |
| | 3) |

### V. OX40 Agonists

Certain embodiments of the present invention comprise an OX40 agonist. The term “OX40 agonist” or “OX40 binding agonist,” as used herein, means, any chemical compound or biological molecule, as defined herein, which upon binding to OX40, (1) stimulates or activates OX40, (2) enhances, increases, promotes, induces, or prolongs an activity, function, or presence of OX40, or (3) enhances, increases, promotes, or induces the expression of OX40. OX40 agonists useful in the any of the treatment method, medicaments and uses of the present invention include a monoclonal antibody (mAb), or antigen binding fragment thereof, which specifically binds to OX40. In any of the treatment method, medicaments and uses of the present invention in which a human individual is being treated, the OX40 agonists increase an OX40-mediated response. In some embodiments of the treatment method, medicaments and uses of the present invention, OX40 agonists markedly enhance cytotoxic T-cell responses, resulting in antitumor activity in several models.

An OX40 agonist includes, for example, an OX40 agonist antibody (e.g., an anti-human OX40 agonist antibody), an OX40L agonist fragment, an OX40 oligomeric receptor, and an OX40 immunoadhesin.

The term “OX40 antibody,” “OX40 agonist antibody,” “anti-OX40 monoclonal antibody,” “αOX40” or “anti-OX40 antibody,” as used herein, means an antibody, as defined herein, capable of binding to OX40 receptor (e.g., human OX40 receptor).

The terms “OX40” and “OX40 receptor” are used interchangeably in the present application, and refer to any form of OX40 receptor, as well as variants, isoforms, and species homologs thereof that retain at least a part of the activity of OX40 receptor. Accordingly, a binding molecule, as defined and disclosed herein, may also bind OX40 from species other than human. In other cases, a binding molecule may be completely specific for the human OX40 and may not exhibit species or other types of cross-reactivity. Unless indicated differently, such as by specific reference to human OX40, OX40 includes all mammalian species of native sequence OX40, e.g., human, canine, feline, equine and bovine. One exemplary human OX40 is a 277 amino acid protein (UniProt Accession No. P43489).

An OX40 agonist antibody, as used herein, means, any antibody, as defined herein, which upon binding to OX40, (1) stimulates or activates OX40, (2) enhances, increases, promotes, induces, or prolongs an activity, function, or presence of OX40, or (3) enhances, increases, promotes, or induces the expression of OX40.

OX40 agonists useful in the any of the treatment method, medicaments and uses of the present invention include a monoclonal antibody (mAb) which specifically binds to OX40 (e.g., anti-OX40 agonist antibody).

In some embodiments, the OX40 agonist antibody increases CD4+ effector T cell proliferation and/or increases cytokine production by the CD4+ effector T cell as compared to proliferation and/or cytokine production prior to treatment with the OX40 agonist antibody. In some embodiments, the cytokine is IFN-γ.

In some embodiments, the OX40 agonist antibody increases memory T cell proliferation and/or increasing cytokine production by the memory cell. In some embodiments, the cytokine is IFN-γ. [0211] In some embodiments, the OX40 agonist antibody inhibits Treg suppression of effector T cell function. In some embodiments, effector T cell function is effector T cell proliferation and/or cytokine production. In some embodiments, the effector T cell is a CD4+ effector T cell.

In some embodiments, the OX40 agonist antibody increases OX40 signal transduction in a target cell that expresses OX40. In some embodiments, OX40 signal transduction is detected by monitoring NFκB downstream signaling.

In some embodiments, the anti-human OX40 agonist antibody is a depleting anti-human OX40 antibody (e.g., depletes cells that express human OX40). In some embodiments, the human OX40 expressing cells are CD4+ effector T cells. In some embodiments, the human OX40 expressing cells are Treg cells. In some embodiments, depleting is by ADCC and/or phagocytosis. In some embodiments, the antibody mediates ADCC by binding FcyR expressed by a human effector cell and activating the human effector cell function. In some embodiments, the antibody mediates phagocytosis by binding FcyR expressed by a human effector cell and activating the human effector cell function. Exemplary human effector cells include, e.g., macrophage, natural killer (NK) cells, monocytes, neutrophils. In some embodiments, the human effector cell is macrophage.

In some embodiments, the anti-human OX40 agonist antibody has a functional Fc region. In some embodiments, effector function of a functional Fc region is ADCC. In some embodiments, effector function of a functional Fc region is phagocytosis. In some embodiments, effector function of a functional Fc region is ADCC and phagocytosis. In some embodiments, the Fc region is human IgG-1. In some embodiments, the Fc region is human IgG-4.

In some embodiments, the anti-human OX40 agonist antibody is a human or humanized antibody.

Examples of OX40 agonist antibody, and useful in the treatment method, medicaments and uses of the present invention, are described in, for example, U.S. Pat. No. 7,960,515, PCT Patent Application Publication Nos. WO 2013/028231 and WO 2013/119202, and U.S. Patent Application Publication No. 2015/0190506.

In some embodiments an anti-OX40 antibody useful in the treatment, method, medicaments and uses disclosed herein is a fully human agonist monoclonal antibody comprising a heavy chain variable region and a light chain variable region comprising the amino acid sequences shown in SEQ ID NO: 54 and SEQ ID NO: 55, respectively. In some embodiments, the anti-OX40 antibody is a fully human IgG-2 or IgG-1 antibody.

Table 6 below provides exemplary anti-OX40 monoclonal antibody sequences for use in the treatment method, medicaments and uses of the present invention.

TABLE 6
EXEMPLARY ANTI-HUMAN OX40 MONOCLONAL ANTIBODY
SEQUENCES

| | |
|---|---|
| CDRH1 | SYSMN (SEQ ID NO: 48) |
| CDRH2 | YISSSSSTIDYADSVKG (SEQ ID NO: 49) |
| CDRH3 | ESGWYLFDY (SEQ ID NO: 50) |
| CDRL1 | RASQGISSWLA (SEQ ID NO: 51) |
| CDRL2 | AASSLQS (SEQ ID NO: 52) |
| CDRL3 | QQYNSYPPT (SEQ ID NO: 53) |
| Heavy chain VR | EVQLVESGGGLVQPGGSLRLSCAASGFTFSSYSMNWV |
| | RQAPGKGLEWVSYISSSSSTIDYADSVKGRFTISRDNAK |
| | NSLYLQMNSLRDEDTAVYYCARESGWYLFDYWGQGTL |
| | VTVSS (SEQ ID NO: 54) |
| Light chain VR | DIQMTQSPSSLSASVGDRVTITCRASQGISSWLAWYQQ |
| | KPEKAPKSLIYAASSLQSGVPSRFSGSGSGTDFTLTISS |
| | LQPEDFATYYCQQYNSYPPTFGGGTKVEIK (SEQ ID |
| | NO: 55) |
| Heavy chain | EVQLVESGGGLVQPGGSLRLSCAASGFTFSSYSMNWV |
| | RQAPGKGLEWVSYISSSSSTIDYADSVKGRFTISRDNAK |
| | NSLYLQMNSLRDEDTAVYYCARESGWYLFDYWGQGTL |
| | VTVSSASTKGPSVFPLAPCSRSTSESTAALGCLVKDYFP |
| | EPVTVSWNSGALTSGVHTFPAVLQSSGLYSLSSVVTVP |
| | SSNFGTQTYTCNVDHKPSNTKVDKTVERKCCVECPPC |
| | PAPPVAGPSVFLFPPKPKDTLMISRTPEVTCVVVDVSHE |
| | DPEVQFNWYVDGVEVHNAKTKPREEQFNSTFRVVSVL |
| | TVVHQDWLNGKEYKCKVSNKGLPAPIEKTISKTKGQPR |
| | EPQVYTLPPSREEMTKNQVSLTCLVKGFYPSDIAVEWE |
| | SNGQPENNYKTTPPMLDSDGSFFLYSKLTVDKSRWQQ |
| | GNVFSCSVMHEALHNHYTQKSLSLSPGK (SEQ ID NO: |
| | 56) |
| Light chain | DIQMTQSPSSLSASVGDRVTITCRASQGISSWLAWYQQ |
| | KPEKAPKSLIYAASSLQSGVPSRFSGSGSGTDFTLTISS |
| | LQPEDFATYYCQQYNSYPPTFGGGTKVEIKRTVAAPSV |
| | FIFPPSDEQLKSGTASVVCLLNNFYPREAKVQWKVDNA |
| | LQSGNSQESVTEQDSKDSTYSLSSTLTLSKADYEKHKV |
| | YACEVTHQGLSSPVTKSFNRGEC (SEQ ID NO: 57) |

### VI. 4-1 BB Agonist

Certain embodiments of the present invention comprise a 4-1BB binding agonist. The term “4-1BB binding agonist” or “4-1BB agonist,” as used herein, means, any chemical compound or biological molecule, as defined herein, which upon binding to 4-1BB, (1) stimulates or activates 4-1BB, (2) enhances, increases, promotes, induces, or prolongs an activity, function, or presence of 4-1BB, or (3) enhances, increases, promotes, or induces the expression of 4-1BB. 4-1BB agonists useful in any of the treatment method, medicaments and uses of the present invention include a monoclonal antibody (mAb), or antigen binding fragment thereof, which specifically binds to 4-1BB. Alternative names or synonyms for 4-1BB include CD137 and TNFRSF9. In any of the treatment method, medicaments and uses of the present invention in which a human individual is being treated, the 4-1BB agonists increase a 4-1BB-mediated response. In some embodiments of the treatment method, medicaments and uses of the present invention, 41BB agonists markedly enhance cytotoxic T-cell responses, resulting in antitumor activity in several models. The term “4-1BB antibody,” “4-1BB agonist antibody,” “anti-4-1BB monoclonal antibody,” “a4-1BB” or “anti-4-1BB antibody,” as used herein, means an antibody, as defined herein, capable of binding to 4-1BB receptor (e.g., human 4-1BB receptor).

The terms “4-1BB” and “4-1BB receptor” are used interchangeably in the present application and refer to any form of 4-1BB receptor, as well as variants, isoforms, and species homologs thereof that retain at least a part of the activity of 4-1BB receptor.

Accordingly, a binding molecule, as defined and disclosed herein, may also bind 4-1BB from species other than human. In other cases, a binding molecule may be completely specific for the human 4-1BB and may not exhibit species or other types of cross-reactivity. Unless indicated differently, such as by specific reference to human 4-1BB. 4-1BB includes all mammalian species of native sequence of 4-1BB, e.g., human, canine, feline, equine and bovine. One exemplary human 4-1BB is a 255 amino acid protein (Accession No. NM_001561; NP_001552).

4-1BB comprises a signal sequence (amino acid residues 1-17), followed by an extracellular domain (169 amino acids), a transmembrane region (27 amino acids), and an intracellular domain (42 amino acids) (Cheuk ATC et al., 2004 Cancer Gene Therapy 11: 215-226). The receptor is expressed on the cell surface in monomer and dimer forms and likely trimerizes with 4-1BB ligand to signal.

Human 4-1BB comprises a signal sequence (amino acid residues 1-17), followed by an extracellular domain (169 amino acids), a transmembrane region (27 amino acids), and an intracellular domain (42 amino acids) (Cheuk A T C et al., Cancer Gene Therapy 2004, 11: 215-226). The receptor is expressed on the cell surface in monomer and dimer forms and likely trimerizes with 4-1BB ligand to signal.

Examples of mAbs that bind to human 4-1BB, and useful in the treatment method, medicaments and uses of the present invention, are described in U.S. Pat. No. 8,337,850 and US20130078240. In some embodiments an anti-4-1BB antibody useful in the treatment, method, medicaments and uses disclosed herein is a fully humanized IgG-2 agonist monoclonal antibody comprising a heavy chain variable region and a light chain variable region comprising the amino acid sequences shown in SEQ ID NO: 64 and SEQ ID NO: 65, respectively.

Table 7 below provides exemplary anti-4-1BB monoclonal antibody sequences for use in the treatment method, medicaments and uses of the present invention.

TABLE 7
EXEMPLARY ANTI-HUMAN 4-IBB MONOCLONAL ANTIBODY
SEQUENCES

| | |
|---|---|
| CDRH1 | STYWIS (SEQ ID NO: 58) |
| CDRH2 | KIYPGDSYTNYSPSFQG (SEQ ID NO: 59) |
| CDRH3 | RGYGIFDY (SEQ ID NO: 60) |
| CDRL1 | SGDNIGDQYAH (SEQ ID NO: 61) |
| CDRL2 | QDKNRPS (SEQ ID NO: 62) |
| CDRL3 | ATYTGFGSLAV (SEQ ID NO: 63) |
| Heavy chain VR | EVQLVQSGAEVKKPGESLRISCKGSGYSFSTYWISWVR |
| | QMPGKGLEWMGKIYPGDSYTNYSPSFQGQVTISADKSI |
| | STAYLQWSSLKASDTAMYYCARGYGIFDYWGQGTLVT |
| | VSS (SEQ ID NO: 64) |
| Light chain VR | SYELTQPPSVSVSPGQTASITCSGDNIGDQYAHWYQQK |
| | PGQSPVLVIYQDKNRPSGIPERFSGSNSGNTATLTISGT |
| | QAMDEADYYCATYTGFGSLAVFGGGTKLTVL (SEQ ID |
| | NO: 65) |
| Heavy chain | EVQLVQSGAEVKKPGESLRISCKGSGYSFSTYWISWVR |
| | QMPGKGLEWMGKIYPGDSYTNYSPSFQGQVTISADKSI |
| | STAYLQWSSLKASDTAMYYCARGYGIFDYWGQGTLVT |
| | VSSASTKGPSVFPLAPCSRSTSESTAALGCLVKDYFPE |
| | PVTVSWNSGALTSGVHTFPAVLQSSGLYSLSSVVTVPS |
| | SNFGTQTYTCNVDHKPSNTKVDKTVERKCCVECPPCP |
| | APPVAGPSVFLFPPKPKDTLMISRTPEVTCVVVDVSHED |
| | PEVQFNWYVDGVEVHNAKTKPREEQFNSTFRVVSVLT |
| | VVHQDWLNGKEYKCKVSNKGLPAPIEKTISKTKGQPRE |
| | PQVYTLPPSREEMTKNQVSLTCLVKGFYPSDIAVEWES |
| | NGQPENNYKTTPPMLDSDGSFFLYSKLTVDKSRWQQG |
| | NVFSCSVMHEALHNHYTQKSLSLSPGK (SEQ ID NO: 66) |
| Light chain | SYELTQPPSVSVSPGQTASITCSGDNIGDQYAHWYQQK |
| | PGQSPVLVIYQDKNRPSGIPERFSGSNSGNTATLTISGT |
| | QAMDEADYYCATYTGFGSLAVFGGGTKLTVLGQPKAA |
| | PSVTLFPPSSEELQANKATLVCLISDFYPGAVTVAWKAD |
| | SSPVKAGVETTTPSKQSNNKYAASSYLSLTPEQWKSHR |
| | SYSCQVTHEGSTVEKTVAPTECS (SEQ ID NO: 67) |

### VII. Methods, Uses and Medicaments

### General Methods

Standard methods in molecular biology are described in Sambrook, Fritsch and Maniatis (1982 & 1989 2nd Edition, 2001 3rd Edition) Molecular Cloning, A Laboratory Manual; Sambrook and Russell Molecular Cloning, 3rd ed., 2001; Wu, Recombinant DNA, Vol. 217. Standard methods also appear in Ausbel, et al., Current Protocols in Molecular Biology, Vols.1-4, 2001, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al., Current Protocols in Protein Science, Vol. 1, 2000, John Wiley and Sons, Inc., New York). Chemical analysis, chemical modification, post-translational modification, production of fusion proteins, and glycosylation of proteins are described (e.g., Coligan, et al., Current Protocols in Protein Science, Vol. 2, 2000; Ausubel, et al., Current Protocols in Molecular Biology, Vol. 3, 2001, pp. 16.0.5-16.22.17; Sigma-Aldrich, Co. Products for Life Science Research, 2001, pp. 45-89; Amersham Pharmacia Biotech (2001) BioDirectory, pp. 384-391). Production, purification, and fragmentation of polyclonal and monoclonal antibodies are described (Coligan, et al., Current Protocols in Immunology, Vol. 1, 2001; Harlow and Lane, Using Antibodies, 1999). Standard techniques for characterizing ligand/receptor interactions are available (e.g., Coligan, et al., Current Protocols in Immunology, Vol. 4, 2001).

Monoclonal, polyclonal, and humanized antibodies can be prepared (e.g., Sheperd and Dean (eds.) Monoclonal Antibodies, 2000; Kontermann and Dubel (eds.) Antibody Engineering, 2001; Harlow and Lane, Antibodies A Laboratory Manual, 1988, pp. 139-243; Carpenter, et al., Non-Fc receptor-binding humanized anti-CD3 antibodies induce apoptosis of activated human T cells, J. Immunol. 2000, 165:6205; He, et al., Humanization and pharmacokinetics of a monoclonal antibody with specificity for both E- and P-selectin, J. Immunol. 1998, 160:1029; Tang et al., Use of a peptide mimotope to guide the humanization of MRK-16, an anti-P-glycoprotein monoclonal antibody , J. Biol. Chem. 1999, 274:27371-27378; Baca et al., Antibody humanization using monovalent phage display, J. Biol. Chem. 1997, 272:10678-10684; Chothia et al., Conformations of immunoglobulin hypervariable regions, Nature 1989, 342:877-883; Foote and Winter Antibody framework residues affecting the conformation of the hypervariable loops, J. Mol. Biol. 1992, 224:487-499; U.S. Pat. No. 6,329,511).

An alternative to humanization is to use human antibody libraries displayed on phage or human antibody libraries in transgenic mice (Vaughan et al., Human antibodies with sub-nanomolar affinities isolated from a large non-immunized phage display library, Nature Biotechnol. 1996, 14:309-314; Barbas , Synthetic human antibodies, Nature Medicine 1995, 1:837-839; Mendez et al., Functional transplant of megabase human immunoglobulin loci recapitulates human antibody response in mice, Nature Genetics 1997, 15:146-156; Hoogenboom and Chames, Natural and designer binding sites made by phage display technology, Immunol. Today 2000, 21:371-377; Barbas et al., Phage Display: A Laboratory Manual, 2001; Kay et al., Phage Display of Peptides and Proteins: A Laboratory Manual, 1996; de Bruin et al., Selection of high-affinity phage antibodies from phage display libraries, Nature Biotechnol. 1999, 17:397-399).

Purification of antigen is not necessary for the generation of antibodies. Animals can be immunized with cells bearing the antigen of interest. Splenocytes can then be isolated from the immunized animals, and the splenocytes can be fused with a myeloma cell line to produce a hybridoma (e.g., Meyaard, L., et. al., LAIR-1, a novel inhibitory receptor expressed on human mononuclear leukocytes, Immunity 1997, 7:283-290; Wright et al., Inhibition of chicken adipocyte differentiation by in vitro exposure to monoclonal antibodies against embryonic chicken adipocyte plasma membranes, Immunity 2000, 13:233-242; Preston, et al., The leukocyte/neuron cell surface antigen OX2 binds to a ligand on macrophages,) Eur. J. Immunol. 1997, 27:1911-1918, Kaithamana et al., Induction of experimental autoimmune Graves' disease in BALB/c mice, J. Immunol. 1999, 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (e.g., Le Doussal et al., Enhanced in vivo targeting of an asymmetric bivalent hapten to double-antigen-positive mouse B cells with monoclonal antibody conjugate cocktails, J. Immunol. 1991, 146:169-175; Gibellini et al., Extracellular HIV-1 Tat protein induces the rapid Ser133 phosphorylation and activation of CREB transcription factor in both Jurkat lymphoblastoid T cells and primary . . . , J. Immunol. 1998160:3891-3898; Hsing and Bishop, Requirement for nuclear factor-κB activation by a distinct subset of CD40-mediated effector functions in B lymphocytes, J. Immunol. 1999, 162:2804-2811; Everts et al., Selective intracellular delivery of dexamethasone into activated endothelial cells using an E-selectin-directed immunoconjugate, J. Immunol.

2002, 168:883-889).

Methods for flow cytometry, including fluorescence activated cell sorting (FACS), are available (e.g., Owens, et al., Flow Cytometry Principles for Clinical Laboratory Practice, 1994; Givan Flow Cytometry, 2nd ed.; 2001; Shapiro, Practical Flow Cytometry, 2003). Fluorescent reagents suitable for modifying nucleic acids, including nucleic acid primers and probes, polypeptides, and antibodies, for use, e.g., as diagnostic reagents, are available (Molecular Probes, Catalogue, 2003; Sigma-Aldrich, Catalogue, 2003.

Standard methods of histology of the immune system are described (e.g., Muller-Harmelink (ed.), Human Thymus: Histopathology and Pathology, 1986; Hiatt, et al., Color Atlas of Histology, 2000; Louis, et al., Basic Histology: Text and Atlas, 2002.

Software packages and databases for determining, e.g., antigenic fragments, leader sequences, protein folding, functional domains, glycosylation sites, and sequence alignments, are available (e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al., A comparison of signal sequence prediction methods using a test set of signal peptides, Bioinformatics 2000, 16: 741-742; Menne, K. M. L., et. al. A comparisonof signal sequence prediction methods using a test set of signal peptides, Bioinformatics 2000, 16, 741-742; Wren, et al., SIGNAL-sequence information and GeNomic AnaLysisComput. Methods Programs Biomed. 2002, 68:177-181; von Heijne, Patterns of amino acids near signal-sequence cleavage sites, Eur. J. Biochem. 1983, 133:17-21; von Heijne, A new method for predicting signal sequence cleavage sites, Nucleic Acids Res. 1986, 14:4683-4690).

### Therapeutic Methods and Uses

The invention further provides therapeutic methods and uses comprising administering to the subject the combinations as described herein, optionally in further combination with other therapeutic or palliative agents.

In one aspect of the invention, the invention provides a method for treating cancer comprising administering to a subject in need thereof an amount of a cyclin dependent kinase (CDK) inhibitor in combination with an amount of a PD-1 axis binding antagonist, wherein the amounts together are effective in treating cancer, and wherein the CDK inhibitor is an inhibitor of CDK4 and CDK6 (CDK4/6 inhibitor), or an inhibitor of CDK2, CDK4 and CDK6 (CDK2/4/6 inhibitor).

In one such embodiment, the invention is related to a method for treating cancer, further comprising administering to the subject an amount of: a. an OX40 agonist; b. a 4-1BB agonist; or c. an OX40 agonist and a 4-1BB agonist; wherein the amounts together are effective in treating cancer. In some embodiments of each of the foregoing, the OX40 agonist is an anti-OX40 antibody. In further embodiments of each of the foregoing, the 4-1BB agonist is an anti-4-BB antibody.

In some embodiments, the treatment results in sustained response in the individual after cessation of the treatment. The methods of this invention may find use in treating conditions where enhanced immunogenicity is desired such as increasing tumor immunogenicity for the treatment of cancer. As such, a variety of cancers may be treated, or their progression may be delayed.

In some embodiments, the individual has cancer that is resistant (has been demonstrated to be resistant) to one or more PD-1 axis binding antagonists. In some embodiments, resistance to PD-1 axis binding antagonist includes recurrence of cancer or refractory cancer. Recurrence may refer to the reappearance of cancer, in the original site or a new site, after treatment. In some embodiments, resistance to PD-1 axis binding antagonist includes progression of the cancer during treatment with the PD-1 axis binding antagonist. In some embodiments, resistance to PD-1 axis binding antagonist includes cancer that does not response to treatment. The cancer may be resistant at the beginning of treatment or it may become resistant during treatment. In some embodiments, the cancer is at early stage or at late stage.

In one embodiment, the PD-1 axis binding antagonist comprises a PD-1 binding antagonist, a PD-L1 binding antagonist, or a PD-L2 binding antagonist. In some such embodiments, the PD-1 axis binding antagonist comprises a PD-1 binding antagonist. In further embodiments of each of the foregoing, the PD-1 binding antagonist inhibits the binding of PD-1 to its ligand binding partners. In specific embodiments, the PD-1 binding antagonist inhibits the binding of PD-1 to PD-LI. In another embodiment, the PD-1 binding antagonist inhibits the binding of PD-1 to PD-L2. In a further embodiment, the PD-1 binding antagonist inhibits the binding of PD-1 to both PD-L1 and PD-L2. In a specific embodiment, the PD-1 binding antagonist is AMP-224. In additional embodiments, the invention provides the PD-1 binding antagonist is an anti-PD-1 antibody. In some embodiments, the anti-PD-1 antibody is a biosimilar, biobetter, or bioequivalent thereof. In a particular embodiment, the anti-PD-1 antibody is nivolumab (MDX 1106), pembrolizumab (MK-3475), pidilizumab (CT-011), cemiplimab (REGN2810), tislelizumab (BGB-A317), spartalizumab (PDR001), RN888, mAb15, MEDI-0680 (AMP-514), BGB-108, or AGEN-2034, or a combination thereof.

In further embodiments of each of the foregoing, the PD-1 axis binding antagonist comprises a PD-L1 binding antagonist. In a particular embodiment, the PD-L1 binding antagonist inhibits the binding of PD-L1 to PD-1. In additional embodiments, the PD-L1 binding antagonist inhibits the binding of PD-L1 to B7-1. In yet another embodiment, the PD-L1 binding antagonist inhibits the binding of PD-L1 to both PD-1 and B7-1.

In specific embodiments, the PD-L1 binding antagonist is an anti-PD-L1 antibody. In some embodiments, the anti-PD-L1 antibody is a biosimilar, biobetter, or bioequivalent thereof. In some embodiments, the anti-PD-L1 antibody is BMS-936559 (MDX-1105), AMP-714, atezolizumab (MPDL3280A), durvalumab (MEDI4736), avelumab, or an antibody comprising a VH region produced by the expression vector with ATCC Accession No. PTA-121183 and having the VL region produced by the expression vector with ATCC Accession No. PTA-121182, or a combination thereof.

In an aspect of the present invention, the OX40 agonist is an anti-OX40 antibody, an OX40L agonist fragment, an OX40 oligomeric receptor, a trimeric OX40L-Fc protein or an OX40 immunoadhesin, or a combination thereof. In some embodiments, the OX40 agonist antibody binds human OX40. In some embodiments, the anti-OX40 antibody is any one of the anti-human OX40 antibodies disclosed herein. In a particular embodiment of each of the foregoing, the OX40 agonist is an anti-OX40 antibody. In some embodiments, the anti-OX40 antibody is a biosimilar, biobetter, or bioequivalent thereof. In one such embodiment, the anti-OX40 antibody is MEDI6469, MEDI0562, MEDI6383, MOXR0916, or GSK3174998, or a combination thereof.

In some embodiments of the each of the foregoing, the anti-OX40 antibody is a full-length human IgG-1 antibody. In a particular embodiment, the OX40 agonist is an OX40L agonist fragment comprising one or more extracellular domains of OX40L.

In yet another aspect, the 4-1BB agonist is an anti-4-1BB antibody. In some embodiments, the anti-4-1BB antibody is a biosimilar, biobetter, or bioequivalent thereof. In a particular embodiment, the 4-1BB agonist is utomilumab (PF-05082566), 1D8, 3EIor, 4B4, H4-1BB-M127, BBK2, 145501, antibody produced by cell line deposited as ATCC No. HB-11248, 5F4, C65-485, urelumab (BMS-663513), 20H4.9-IgG-1 (BMS-663031), 4E9, BMS-554271, BMS-469492, 3H3, BMS- 469497, 3E1, 53A2, or 3B8.

In one aspect, the antibody against PD-L1, PD-1, OX40, and/or 4-1BB may incorporated into a multi-specific antibody (e.g., a bispecific antibody). In some such embodiments, a bispecific antibody comprises a first antibody variable domain and a second antibody variable domain, wherein the first antibody variable domain is capable of recruiting the activity of a human immune effector cell by specifically binding to an effector antigen located on the human immune effector cell, and wherein the second antibody variable domain is capable of specifically binding to a target antigen as provided herein. In some embodiments, the antibody has an IgG1, IgG2, IgG3, or IgG4 isotype. In some embodiments, the antibody comprises an immunologically inert Fc region. In some embodiments the antibody is a human antibody or humanized antibody.

In some embodiments, the bispecific antibody provided herein binds to two different target antigens on the same target cell (e.g., two different antigens on the same tumor cell). Such antibodies may be advantageous, for example, for having increased specificity for a target cell of interest (e.g., for a tumor cell that expresses two particular tumor associated antigens of interest). For example, in some embodiments, a bispecific antibody provided herein comprises a first antibody variable domain and a second antibody variable domain, wherein the first antibody variable domain is capable of specifically binding to a first target antigen as provided herein and the second antibody variable domain is capable of specifically binding to a second target antigen as provided herein. In some embodiments, the first target antigen is PD-L1 and the second target antigen is CD47. Examples of mAbs that bind to human PD-L1 and that may be used in bispecific anti-PD-L1/anti-CD47 antibodies include antibodies described in WO 2013/079174, WO 2015/061668, WO 2010/089411, WO 2007/005874, WO 2010/036959, WO 2014/100079, WO 2013/019906, WO 2010/077634, and U.S. Pat. Nos. 8,552,154, 8779,108, and 8,383,796. Examples of mAbs that bind to CD47 and that may be used in bispecific anti-PD-L1/anti-CD47 antibodies include the anti-CD47 antibodies Hu5F9-G4 (Forty Seven Inc.), CC-90002 (Celgene), SRF231, and B6H12.

Methods for making bispecific antibodies are known in the art (e.g., c). Traditionally, the recombinant production of bispecific antibodies was based on the coexpression of two immunoglobulin heavy chain-light chain pairs, with the two heavy chains having different specificities (Millstein and Cuello, Hybrid hybridomas and their use in immunohistochemistry, Nature 1983, 305, 537-539).

In an aspect of the present invention, the CDK inhibitor is a CDK4/6 inhibitor. In one such embodiment, the CDK4/6 inhibitor is palbociclib, or a pharmaceutically acceptable salt thereof.

In another aspect, the CDK inhibitor is a CDK2/4/6 inhibitor. In some such embodiments, the CDK2/4/6 inhibitor is 6-(difluoromethyl)-8-((1R,2R)-2-hydroxy-2-methylcyclopentyl)-2-(1-(methylsulfonyl)piperidin-4-ylamino)pyrido[2,3-d]pyrim idin-7(8H)-one, or a pharmaceutically acceptable salt thereof.

In one aspect, the invention provides a method for treating a cancer in a subject comprising administering to the subject a combination therapy of the invention. In one aspect, the invention provides a method for treating a cancer comprising administering to a subject in need thereof an amount of a cyclin dependent kinase (CDK) inhibitor and an amount of a PD-1 axis binding antagonist, wherein the amounts together are effective in treating cancer, and wherein the CDK inhibitor is an inhibitor of CDK4 and CDK6 (CDK4/6 inhibitor), or an inhibitor of CDK2, CDK4 and CDK6 (CDK2/4/6 inhibitor). In some such embodiments the subject is a human.

In some embodiments, the method involves the use of an inhibitor of CDK4 and CDK6 (CDK4/6 inhibitor), or an inhibitor of CDK2, CDK4 and CDK6 (CDK2/4/6 inhibitor) in combination with an anti-PD-L1 antibody.

In some embodiments, the method involves the use of an inhibitor of CDK4 and CDK6 (CDK4/6 inhibitor), or an inhibitor of CDK2, CDK4 and CDK6 (CDK2/4/6 inhibitor) in combination with an anti-PD-L1 antibody and an anti-OX40 antibody.

In some embodiments, the method involves the use of an inhibitor of CDK4 and CDK6 (CDK4/6 inhibitor), or an inhibitor of CDK2, CDK4 and CDK6 (CDK2/4/6 inhibitor) in combination with an anti-PD-L1 antibody and an anti-4-1 BB antibody.

In some embodiments, the method involves the use of an inhibitor of CDK4 and CDK6 (CDK4/6 inhibitor), or an inhibitor of CDK2, CDK4 and CDK6 (CDK2/4/6 inhibitor) in combination with an anti-PD-L1 antibody, an anti-OX40 antibody and an anti-4-1 BB antibody.

In some embodiments, the method involves the use of palbociclib, or a pharmaceutically acceptable salt thereof, in combination with avelumab.

In some embodiments, the method involves the use of 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof, in combination with avelumab.

In some embodiments, the method involves the use of palbociclib, or a pharmaceutically acceptable salt thereof, in combination with avelumab and an anti-OX40 antibody.

In some embodiments, the method involves the use of 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof, in combination with avelumab and an anti-OX40 antibody.

In some embodiments, the method involves the use of palbociclib, or a pharmaceutically acceptable salt thereof, in combination with avelumab and utomilumab.

In some embodiments, the method involves the use of 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof in combination with avelumab and utomilumab.

In some embodiments, the method involves the use of palbociclib, or a pharmaceutically acceptable salt thereof, in combination with avelumab, anti-OX40 antibody and utomilumab.

In some embodiments, the method involves the use of 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof, in combination with avelumab, anti-OX40 antibody and utomilumab.

In some embodiments, the OX40 agonist in the combination therapy comprises an anti-OX40 antibody comprising: a heavy chain variable region (VH) comprising a heavy chain complementarity determining region one (CDRH1), a heavy chain complementarity determining region two (CDRH2), a heavy chain complementarity determining region three (CDRH3), comprising the amino acid sequence shown in SEQ ID NO: 48, SEQ ID NO: 49 and SEQ ID NO: 50; and a light chain variable region (VL) comprising a light chain complementarity determining region one (CDRL1), a light chain complementarity determining region two (CDRL2), and a light chain complementarity determining region three (CDRL3), comprising the amino acid sequence shown in SEQ ID NO: 51; SEQ ID NO: 52 and SEQ ID NO: 53.

In specific embodiments of each of the aspects described herein, the anti-OX40 antibody comprises the CDRH1 comprising the amino acid sequence shown in SEQ ID NO: 48, the CDRH2 comprising the amino acid sequence shown in SEQ ID NO: 49, and the CDRH3 comprising the amino acid sequence shown in SEQ ID NO: 50; and/or the CDRL1 comprising the amino acid sequence shown in SEQ ID NO: 51, the CDRL2 comprising the amino acid sequence shown in SEQ ID NO: 52, and the CDRL3 comprising the amino acid sequence shown in SEQ ID NO: 53.

In specific embodiments of each of the aspects described herein, the anti-OX40 antibody comprises a VH and a VL, wherein the VH and the VL comprise SEQ ID NO: 54 and SEQ ID NO: 55, respectively.

In some embodiments, the 4-1 BB agonist in the combination therapy comprises an anti-4-1 BB monoclonal antibody comprising: a VH comprising a CDRH1, a CDRH2, a CDRH3, comprising the amino acid sequence shown in SEQ ID NO: 58, SEQ ID NO: 59 and SEQ ID NO: 60; and a VL comprising a CDRL1, a CDRL2, and a CDRL3, comprising the amino acid sequence shown in SEQ ID NO: 61; SEQ ID NO: 62 and SEQ ID NO: 63.

In specific embodiments of each of the aspects described herein, the anti-4-1BB antibody comprises the CDRH1 comprising the amino acid sequence shown in SEQ ID NO: 58, the CDRH2 comprising the amino acid sequence shown in SEQ ID NO: 59, and the CDRH3 comprising the amino acid sequence shown in SEQ ID NO: 60; and/or the CDRL1 comprising the amino acid sequence shown in SEQ ID NO: 61, the CDRL2 comprising the amino acid sequence shown in SEQ ID NO: 62, and the CDRL3 comprising the amino acid sequence shown in SEQ ID NO: 63.

In some specific embodiments, the 4-1BB agonist in the combination therapy comprises an anti-4-1BB monoclonal antibody comprising a VH and a VL comprising the amino acid sequences shown in SEQ ID NO: 64 and SEQ ID NO: 65, respectively.

In some embodiments of the each of the foregoing, the cancer is a solid tumor. In yet another embodiment, the cancer is a hematologic cancer.

In a further embodiment, the invention is related to a method for treating cancer, wherein the cancer is selected from the group consisting of brain cancer, head/neck cancer (including squamous cell carcinoma of the head and neck (SCCHN)), prostate cancer, ovarian cancer, bladder cancer (including urothelial carcinoma, also known as transitional cell carcinoma (TCC)), lung cancer (including squamous cell carcinoma, small cell lung cancer (SCLC), and non-small cell lung cancer (NSCLC)), breast cancer, bone cancer, colorectal cancer, kidney cancer, liver cancer (including hepatocellular carcinoma (HCC)), stomach cancer, pancreatic cancer, esophageal cancer, cervical cancer, sarcoma, skin cancer (including melanoma and Merkel cell carcinoma (MCC)), multiple myeloma, mesothelioma, malignant rhabdoid tumors, neuroblastoma, diffuse intrinsic pontine glioma (DIPG), carcinoma, lymphoma, diffuse large B-cell lymphoma (DLBCL), primary mediastinal B-cell lymphoma (PMBCL), follicular lymphoma, acute lymphoblastic leukemia (ALL), acute myeloid leukemia (AML), chronic lymphocytic leukemia (CLL), chronic myeloid leukemia (CML), follicular lymphoma, Hodgkin's lymphoma (HL), classical Hodgkin lymphoma (cHL), mantle cell lymphoma (MCL), multiple myeloma (MM), myeloid cell leukemia-1 protein (Mcl-1), myelodysplastic syndrome (MDS), non-Hodgkin's lymphoma (NHL), small lymphocytic lymphoma (SLL), and SWI/SNF-mutant cancer.

In some embodiments, the methods may further comprise an additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, phototherapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery.

The CDK inhibitor, the PD-1 axis binding antagonist, OX40 agonist and/or 4-1BB agonist may be administered by the same route of administration or by different routes of administration.

An effective amount of the CDK inhibitor and PD-1 axis binding antagonist, OX40 agonist and/or 4-1BB agonist may be administered for prevention or treatment of disease. The appropriate dosage of the CDK inhibitor and PD-1 axis binding antagonist, OX40 agonist and/or 4-1BB agonist may be determined based on the type of disease to be treated, the type of the CDK inhibitor, PD-1 axis binding antagonist, OX40 agonist and/or 4-1BB agonist, the severity and course of the disease, the clinical condition of the subject, the subject's clinical history and response to the treatment, and the discretion of the attending physician.

In some embodiments of the methods, uses, compositions, and kits described above and herein, the treatment further comprises administering a chemotherapeutic agent for treating or delaying progression of cancer in a subject. In some embodiments, the subject has been treated with a chemotherapeutic agent before the combination treatment with the CDK inhibitor, PD-1 axis binding antagonist, the OX40 binding agonist and/or the 4-1BB agonist. In some embodiments, the subject treated with the combination of the CDK inhibitor, PD-1 axis binding antagonist, the OX40 binding agonist and/or the 4-1BB agonist is refractory to a chemotherapeutic agent treatment. Some embodiments of the methods, uses, compositions, and kits described throughout the application, further comprise administering a chemotherapeutic agent for treating or delaying progression of cancer.

In some embodiments, the combination therapy of the invention comprises administration of a CDK inhibitor in combination with a PD-1 axis binding antagonist, and optionally additionally in combination with an OX40 agonist (e.g., anti- human OX40 antibody) and/or a 4-1BB agonist (anti human 4-1BB antibody). In the methods provided herein, each of the CDK inhibitor, PD-1 axis binding antagonist, OX40 agonist and/or 4-1BB agonist may be administered in any suitable manner known in the art. In one embodiment, the CDK inhibitor and the PD-1 axis binding antagonist are administered simultaneously or sequentially in either order. In a further embodiment, the CDK inhibitor, the PD-1 axis binding antagonist, and the OX40 agonist are administered simultaneously or sequentially in any order. In a further embodiment, the CDK inhibitor, the PD-1 axis binding antagonist, the OX40 agonist and the 4-1BB agonist are administered simultaneously or sequentially in any order.

In additional embodiments, the CDK inhibitor, the PD-1 axis binding antagonist and the 4-1BB agonist are administered simultaneously or sequentially in any order. In yet another embodiment, the CDK inhibitor, the PD-1 axis binding antagonist, the OX40 agonist and the 4-1BB agonist are administered simultaneously or sequentially in any order.

In some embodiments of each of the foregoing, the PD-1 axis binding antagonist is: a PD-1 binding antagonist; a PD-L1 binding antagonist; or a PD-1 binding antagonist and a PD-L1 binding antagonist.

In some embodiments of the each of the foregoing, a. the PD-1 binding antagonist and the PD-L1 binding antagonist are in the same composition; b. the PD-1 binding antagonist and the OX40 agonist are in the same composition; c. the PD-1 binding antagonist and the 4-1BB agonist are in the same composition; d. the PD-L1 binding antagonist and the OX40 agonist are in the same composition; e. the PD-L1 binding antagonist and the 4-1BB agonist are in the same composition; f. the OX40 agonist and the 4-1BB agonist are in the same composition; g. the PD-1 binding antagonist, the PD-L1 binding antagonist and the OX40 agonist are in the same composition; h. the PD-1 binding antagonist, the PD-L1 binding antagonist and the 4-1BB agonist are in the same composition; i. the PD-1 binding antagonist, the OX40 agonist and the 4-1BB agonist are in the same composition; j. the PD-L1 binding antagonist, the OX40 agonist and the 4-1BB agonist are in the same composition; or k. the PD-1 binding antagonist, the PD-L1 binding antagonist, the OX40 agonist and the 4-1BB agonist are in the same composition.

### VIII. Dosage Forms and Regimens

Administration of the compounds of the invention may be affected by any method that enables delivery of the compounds to the site of action. These methods include oral routes, intraduodenal routes, parenteral injection (including intravenous, subcutaneous, intramuscular, intravascular or infusion), topical, and rectal administration.

Those skilled in the art will be able to determine the appropriate amount, dose or dosage of each compound, as used in the combination of the present invention, to administer to a patient, taking into account variety of factors, including, though not limited to, the degree of advancement of the disease, age, weight, general health, gender, diet, the compound administered, the time and route of administration, the nature and advancement of cancer, requiring treatment, and other medications the individual is taking.

In some embodiments, the methods of administration of the agents and combinations herein may include oral, intravenous, intramuscular subcutaneous, topical, transdermal, intraperitoneal, intraorbital, by implantation, by inhalation, intrathecal, intraventricular, or intranasal administration.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

For combination therapies as described herein, the agents may be administered at their approved dosages. Treatment is continued as long as clinical benefit is observed or until unacceptable toxicity or disease progression occurs. Nevertheless, in certain embodiments, the combination therapies of the present invention may advantageously utilize lower dosages of the administered therapeutic agents, thus avoiding possible toxicities or complications associated with the various monotherapies. For example, the dosages of the agents administered are significantly lower than the approved dosage, e.g., a subtherapeutic dosage of the CDK2/4/6 inhibitor is administered in combination with a subtherapeutic dosage of a PD-1 axis binding antagonist, an OX40 agonist and/or a 4-1BB agonist. It will be appreciated by the skilled practitioner that when the agents of the invention are used as part of a combination therapy, a lower dosage of the agent may be desirable than when the agent alone is administered to a subject, a synergistic therapeutic effect may be achieved through the use of combination therapy which, in turn, permits use of a lower dose of the agent to achieve the desired therapeutic effect.

In one embodiment, the dosages may be lower and may also be applied less frequently, which may diminish the incidence or severity of side-effects. This is in accordance with the desires and requirements of the subjects to be treated.

It is one objective of this invention to provide a pharmaceutical composition comprising an amount, which may be jointly therapeutically effective at treating cancer. In this composition, two or more compounds may be administered together, one after the other or separately in one combined unit dosage form or in two separate unit dosage forms.

The unit dosage form may also be a fixed combination. It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

The amount of the agent of the invention administered will be dependent on the subject being treated, the severity of the disorder or condition, the rate of administration, the disposition of the compound and the discretion of the prescribing physician.

An effective amount of the CDK inhibitor, PD-1 axis binding antagonist, OX40 agonist and/or 4-BB agonist may be administered for prevention or treatment of disease. The appropriate dosage of the CDK inhibitor, PD-1 axis binding antagonist, OX40 agonist and/or 4-BB agonist (e.g., anti-human OX40 agonist antibody) may be determined based on the type of disease to be treated, the type of the CDK inhibitor, PD-1 axis binding antagonist, the OX40 agonist and/or 4-BB agonist, the severity and course of the disease, the clinical condition of the subject, the subject's clinical history and response to the treatment, and the discretion of the attending physician. In some embodiments, combination treatment with CDK inhibitor, PD-1 axis binding antagonist (e.g., anti- PD-1 antibody or anti-PD-L1 antibody), OX40 agonist (e.g., anti-human OX40 agonist antibody) and/or 4-BB agonist (e.g., anti-human 4-1BB agonist antibody) are synergistic, whereby an efficacious dose of the CDK inhibitor, PD-1 axis binding antagonist, OX40 agonist and/or 4-BB agonist in the combination is reduced relative to efficacious dose of the each of the CDK inhibitor, PD-1 axis binding antagonist, OX40 agonist and/or 4-1BB agonist as a single agent.

Dosage units for a PD-1 axis binding antagonist (e.g., pembrolizumab, nivolumab, avelumab) may be expressed as a flat dose, i.e., 100 mg, 200 mg, 300 mg, or as a patient-specific dose, i.e., mg/kg (mg therapeutic agent/kg of body weight) or mg/m² (quantity in milligrams per square meter of body surface area).

As a general proposition, the therapeutically effective amount of the antibody administered to human will be in the range of about 0.01 to about 50 mg/kg of patient body weight whether by one or more administrations. In some embodiments, the antibody used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some embodiments, the antibody is administered at 15 mg/kg. However, other dosage regimens may be useful. For example, in some embodiments, an anti-PD-L1 antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The dose of the antibody administered in a combination treatment may be reduced as compared to a single treatment. The progress of this therapy is easily monitored by conventional techniques.

In some embodiments that employ an antibody, antibody fragment or fusion soluble receptor as the PD-1 axis binding antagonist in the combination therapy, may comprise administering the antibody at a dose of about 0.5, 1, 2, 3, 5 or 10 mg/kg at intervals of about 7 days (±2 days) or 14 days (±2 days) or about 21 days (±2 days) or about 30 days (±2 days) throughout the course of treatment. Alternately, in some embodiments that employ an antibody, antibody fragment or fusion soluble receptor as the PD-1 axis binding antagonist in the combination therapy, the dosing regimen will comprise administering the antibody a dose of from about 0.005 mg/kg to about 10 mg/kg, with intrapatient dose escalation. In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (±2 days), for doses subsequent to the second dose. In certain embodiments, the dosing interval will be about 7 days (±2 days), for doses subsequent to the second dose.

In certain embodiments, a subject will be administered an intravenous (IV) infusion of a medicament comprising any of the PD-1 axis binding antagonists described herein.

In one embodiment of the invention, the PD-1 axis binding antagonist in the combination therapy is nivolumab, pembrolizumab or avelumab (MSB0010718C), which is administered intravenously or in a liquid dosage form at a dose selected from the group consisting of any one of: 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg Q3W.

In some embodiments, pembrolizumab is administered at a dose of 2 mg/kg (up to 200 mg) every 3 weeks. In some embodiments, avelumab is administered at a dose of 10 mg/kg as an intravenous infusion over 60 minutes every 2 weeks. In some embodiments, the optimal dose for a PD-1 axis binding antagonist in combination with a CDK inhibitor may be identified by dose escalation of one or both of these agents. The CDK inhibitor may be administered orally (PO), either once daily (QD) or twice daily (BID), with or without food on a continuous or intermittent schedule starting on Cycle 1 Day 1, except in the case of CDK inhibitor lead-in. A PD-1 axis binding antagonist such as avelumab may be administered as a 30-minute to 1-hr intravenous (IV) infusion every 2 weeks (Q2W), every 3 weeks (Q3W) or in case of dose reduction, every 4 weeks (Q4W), starting on Cycle 1 Day 1, except in the case of CDK inhibitor lead-in. On the day of CDK inhibitor administration, the CDK inhibitor may be given prior to or after administration of the PD-1 axis binding antagonist. In another embodiment, an CDK inhibitor can be administered at 1 mg, 2 mg, 5 mg, 10 mg, 15 mg, 20 mg, 25 mg, 30 mg, 35 mg, 40 mg, 45 mg, 50 mg, 75 mg, 100 mg, 125 mg, 150 mg, 200 mg, or 250 mg on a BID or QD schedule, which may be administered continuously or on an intermittent dosing schedule, such as 3 weeks on:1 week off (3:1) or 2 weeks on:1 week off (2:1) schedule, and the PD-1 axis binding antagonist is administered at a starting dose of 2 mg/kg, or 5 mg/kg or 10 mg/kg, at a dosing interval of Q2W, Q3W or alternately Q4W.

In one embodiment, the CDK inhibitor is administered at 25 mg, 50 mg, 75 mg, 100 mg or 125 mg BID or QD for a 3-week lead-in period and then the PD-1 axis binding antagonist is administered at a starting dose of 2 mg/kg Q3W or 200 mg Q3W after the lead-in period. In another embodiment, the CDK inhibitor is administered at 25 mg, 50 mg, 75 mg, 100 mg or 125 mg BID or QD and the PD-1 axis binding antagonist is administered at a starting dose of 2 mg/kg Q4W. In another embodiment, the CDK inhibitor is administered at 25 mg, 50 mg, 75 mg, 100 or 125 mg BID or QD and PD-1 axis binding antagonist is administered at a starting dose of 2 mg/kg Q3W. In another embodiment, the CDK inhibitor is administered at 25 mg, 50 mg, 75 mg, 100 mg or 125 mg BID or QD and the PD-1 axis binding antagonist is administered at a starting dose of 2 mg/kg Q4W. In another embodiment, the CDK inhibitor is administered at 25 mg, 50 mg, 75 mg, 100 mg or 125 mg BID or QD and RN888 is administered at a starting dose of 2 mg/kg Q3W. In another embodiment, the CDK inhibitor is administered at 25 mg, 50 mg, 75 mg, 100 mg or 125 mg BID or QD and the PD-1 axis binding antagonist is administered at a starting dose of 2 mg/kg Q4W. In another embodiment, the CDK inhibitor is administered at 1 mg, 2 mg, 5 mg, 10 mg, 15 mg, 20 mg, 25 mg, 30 mg, 35 mg, 40 mg, 45 mg or 50 mg BID or QD for a 3-week_lead-in period and then the PD-1 axis binding antagonist is administered at a starting dose of 2 mg/kg Q3W or 200 mg Q3W after the lead-in period. In another embodiment, the CDK inhibitor is administered at 1 mg, 2 mg, 5 mg, 10 mg, 15 mg, 20 mg, 25 mg, 30 mg, 35 mg, 40 mg, 45 mg or 50 mg BID or QD and the PD-1 axis binding antagonist is administered at a starting dose of 2 mg/kg Q4W. In another embodiment, the CDK inhibitor is administered at 1 mg, 2 mg, 5 mg, 10 mg, 15 mg, 20 mg, 25 mg, 30 mg, 35 mg, 40 mg, 45 mg or 50 mg BID or QD and PD-1 axis binding antagonist is administered at a starting dose of 2 mg/kg Q3W. In another embodiment, the CDK inhibitor is administered at 1 mg, 2 mg, 5 mg, 10 mg, 15 mg, 20 mg, 25 mg, 30 mg, 35 mg, 40 mg, 45 mg or 50 mg BID or QD and the PD-1 axis binding antagonist is administered at a starting dose of 2 mg/kg Q4W. In another embodiment, the CDK inhibitor is administered at 1 mg, 2 mg, 5 mg, 10 mg, 15 mg, 20 mg, 25 mg, 30 mg, 35 mg, 40 mg, 45 mg or 50 mg BID or QD and RN888 is administered at a starting dose of 2 mg/kg Q3W. In another embodiment, the CDK inhibitor is administered at 1 mg, 2 mg, 5 mg, 10 mg, 15 mg, 20 mg, 25 mg, 30 mg, 35 mg, 40 mg, 45 mg or 50 mg BID or QD and the PD-1 axis binding antagonist is administered at a starting dose of 2 mg/kg Q4W. In some such embodiments, the CDK inhibitor is palbociclib, or a pharmaceutically acceptable salt thereof. In other such embodiments, the CDK inhibitor is PF-06873600, or a pharmaceutically acceptable salt thereof. In a specific embodiment, avelumab is administered at a dose of 10 mg/kg as an intravenous infusion over 60 minutes every 2 weeks in combination with the agents as described herein, until disease progression or unacceptable toxicity.

In a specific embodiment, pembrolizumab is administered at a dose of 200 mg administered as an intravenous infusion over 30 minutes every 3 weeks until disease progression or unacceptable toxicity, or up to 24 months in patients without disease progression. In some embodiments, the subject is treated with a 3-week lead-in period of single agent CDK inhibitor directly preceding the combination administration of the CDK inhibitor and PD-1 axis binding antagonist.

In some embodiments, the patient is treated with a 3-week lead-in period of single-agent CDK inhibitor directly preceding the combination administration of the CDK inhibitor and a PD-1 axis binding antagonist, an OX40 agonist and/or a 4-1 BB agonist.

In some embodiments, a treatment cycle begins with the first day of combination treatment and last for 3 weeks. In such embodiments, the combination therapy is preferably administered for at least 18 weeks (6 cycles of treatment), more preferably at least 24 weeks (8 cycles of treatment), and even more preferably at least 2 weeks after the patient achieves a CR.

In some embodiments of combination therapy described herein, the OX40 agonist is administered about every one, two, three, four, five, or six weeks at: a) a fixed dose per subject selected from the group consisting of about 0.1, 0.5, 1, 2, 4, 5, 6, 8, 10, 20, 30, 40, 50, 60, 70, 80, 90, 100, 150, 200, 300, 400, 500, 600, 700, 800, 900, or 1000 mg, or b) a dose selected from the group consisting of about 0.01 mg/kg, 0.03 mg/kg, 0.1 mg/kg, 0.3 mg/kg, 1 mg/kg, 1.5 mg/kg, 3 mg/kg, 5 mg/kg, 10 mg/kg, 15 mg/kg, 20 mg/kg and 25 mg/kg, and the 4-1 BB agonist is administered about every one, two, three, four, five, or six weeks at: a) a fixed dose per subject selected from the group consisting of about 0.1, 0.5, 1 , 2, 4, 5, 6, 8, 10, 20, 30, 40, 50, 60, 70, 80, 90, 100, 150, 200, 300, 400, 500, 600, 700, 800, 900, or 1000 mg, or b) a dose selected from the group consisting of about 0.01 mg/kg, 0.03 mg/kg, 0.1 mg/kg, 0.3 mg/kg, 1 mg/kg, 1.5 mg/kg, 3 mg/kg, 5 mg/kg, 10 mg/kg, 15 mg/kg, 20 mg/kg and 25 mg/kg.

In specific embodiments, the anti-4-1BB monoclonal antibody is administered at a dose selected from the group consisting of 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg Q3W. In some other embodiments, the anti-4-1BB monoclonal antibody is administered as a liquid medicament, and the selected dose of the medicament is administered by IV infusion over a time period of about 60 minutes.

In some embodiments, the anti-4-1BB monoclonal antibody is administered at a starting dose of about 0.6 mg/kg Q4W and avelumab is administered at a starting dose of 10 mg/kg Q2W, and if the starting dose combination is not tolerated by the subject, then the dose of avelumab is reduced to 5 mg/kg Q2W and/or the dose of the anti-4-1BB monoclonal antibody is reduced to 0.3 mg/kg Q4W.

An effective dosage of a CDK inhibitor, or a pharmaceutically acceptable salt thereof, is in the range of from about 0.001 to about 100 mg per kg body weight per day, preferably about 1 to about 35 mg/kg/day, in single or divided doses. For example, for a 70 kg human, this would amount to about 0.01 to about 7 g/day, preferably about 0.02 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

In some embodiments, the dose of CDK inhibitor is increased up to a maximum dose of 250 mg BID if the subject tolerates the combination treatment at a lower total dose of CDK inhibitor.

In some embodiments, the CDK inhibitor, or a pharmaceutically acceptable salt thereof, is administered at a daily dosage of from about 5 mg to about 250 mg per day, preferably from about 10 mg to about 125 mg per day. In some embodiments, the CDK inhibitor, or a pharmaceutically acceptable salt thereof, is administered at a daily dosage of about 5 mg per day, about 10 mg per day, about 15 mg per day, about 20 mg per day, about 25 mg per day, about 30 mg per day, about 35 mg per day, about 40 mg per day, about 45 mg per day, about 50 mg per day, about 75 mg per day, about 100 mg per day, about 125 mg per day, about 150 mg per day, about 200 mg per day, or about 250 mg per day. This dose may optionally be sub-divided into small doses, for example a dosage of 150 mg per day could be dosed as 75 mg dose twice per day.

Dosage units for a CDK inhibitor (e.g., PF-06873600 or palbociclib) may be expressed as a flat dose, i.e., 1 mg, 2 mg, 5 mg, 10 mg, 15 mg, 20 mg, 25 mg, 30 mg, 35 mg, 40 mg, 45 mg, 50 mg, 75 mg, 100 mg, 125 mg, etc. or as a subject-specific dose, i.e., mg/kg (mg therapeutic agent/kg of body weight) or mg/m²(quantity in milligrams per square meter of body surface area).

Some embodiments may comprise administering the CDK inhibitor in a dose of about: 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, or more than 250 mg, wherein the amounts can be administered once a day (q.d.), twice a day (b.i.d), three times a day (t.i.d.), four times a day (q.i.d.), or on some other dosing schedule.

Repetition of the administration or dosing regimens, or adjustment of the administration or dosing regimen may be conducted as necessary to achieve the desired treatment. A “continuous dosing schedule,” as used herein, is an administration or dosing regimen without dose interruptions, e.g., without days off treatment. Repetition of 21- or 28-day treatment cycles without dose interruptions between the treatment cycles is an example of a continuous dosing schedule. In an embodiment, the compounds of the combination of the present invention can be administered in a continuous dosing schedule. In other embodiments, the CDK inhibitor is administered on an intermittent dosing schedule, such as a 3:1 or 2:1 schedule.

In some such embodiments, the CDK inhibitor is a CDK4/6 inhibitor or a pharmaceutically acceptable salt thereof. In one such embodiment, the CDK4/6 inhibitor is palbociclib, or a pharmaceutically acceptable salt thereof.

In another embodiment, the CDK inhibitor is a CDK2/4/6 inhibitor or a pharmaceutically acceptable salt thereof. In a specific embodiment the CDK2/4/6 inhibitor is 6-(difluoromethyl)-8-((1R,2R)-2-hydroxy-2-methylcyclopentyl)-2-(1-(methylsulfonyl)-piperidin-4-ylamino)pyrido[2,3-d]pyrimidin-7(8H)-one, or a pharmaceutically acceptable salt thereof.

In an embodiment, palbociclib, or a pharmaceutically acceptable salt thereof, is administered at a daily dosage of about 5 mg to about 125 mg once daily, about 5 mg to about 100 mg once daily, 5 mg to about 75 mg once daily, or about 5 mg to about 50 mg once daily. In an embodiment, which is the recommended starting dose or standard clinical dose, palbociclib, or a pharmaceutically acceptable salt thereof, is administered at a daily dosage of about 125 mg once a day. In an embodiment, palbociclib, or a pharmaceutically acceptable salt thereof, is administered at a non-standard clinical dose. In an embodiment, a non-standard clinical dose is a low-dose amount of palbociclib, or a pharmaceutically acceptable salt thereof. For example, palbociclib, or a pharmaceutically acceptable salt thereof, is administered at a dose of about 100 mg once daily, about 75 mg once daily, or about 50 mg once daily. In an embodiment, palbociclib, or a pharmaceutically acceptable salt thereof, is administered ata dose of about 100 mg once daily. In an embodiment, palbociclib, or a pharmaceutically acceptable salt thereof, is administered at a dose of about 75 mg once daily. In an embodiment, palbociclib, or a pharmaceutically acceptable salt thereof, is administered at a dose of about 50 mg once daily. Dosage amounts provided herein refer to the dose of the free base form of palbociclib, or are calculated as the free base equivalent of an administered palbociclib salt form. For example, a dosage or amount of palbociclib, such as 100 mg, 75 mg or 50 mg, refers to the free base equivalent. This dosage regimen may be adjusted to provide the optimal therapeutic response. For example, the dose may be proportionally reduced or increased as indicated by the exigencies of the therapeutic situation.

In an embodiment, PF-06873600, or a pharmaceutically acceptable salt thereof, is administered at a daily dosage of about 5 mg to about 125 mg daily, about 5 mg to about 100 mg daily, about 5 mg to about 75 mg daily, or about 5 mg to about 50 mg daily. In an embodiment, PF-06873600, or a pharmaceutically acceptable salt thereof, is administered at a daily dosage of about 10 mg, about 15 mg, about 25 mg, about 30 mg, about 50 mg, about 75 mg, about 100 mg or about 125 mg daily. In an embodiment, PF-06873600, or a pharmaceutically acceptable salt thereof, is administered at a non-standard clinical dose. In an embodiment, a non-standard clinical dose is a low-dose amount of PF-06873600, or a pharmaceutically acceptable salt thereof. For example, PF-06873600, or a pharmaceutically acceptable salt thereof, is administered at a dose of about 100 mg daily, about 75 mg daily, about 50 mg daily, about 30 mg daily, about 25 mg daily, about 15 mg daily, or about 10 mg daily. In an embodiment, PF-06873600, or a pharmaceutically acceptable salt thereof, is administered at a dose of about 50 mg daily. In an embodiment, PF-06873600, or a pharmaceutically acceptable salt thereof, is administered at a dose of about 30 mg daily. In an embodiment, PF-06873600, or a pharmaceutically acceptable salt thereof, is administered at a dose of about 25 mg daily. Dosage amounts provided herein refer to the dose of the free base form of PF-06873600, or a pharmaceutically acceptable salt thereof, calculated as the free base equivalent of an administered PF-06873600 salt form. For example, a dosage or amount of PF-06873600, such as 100 mg, 75 mg, 50 mg, 30 mg, 25 mg, 15 mg or 10 mg, refers to the free base equivalent. This dosage regimen may be adjusted to provide the optimal therapeutic response. For example, the dose may be proportionally reduced or increased as indicated by the exigencies of the therapeutic situation.

The practice of the method of this invention may be accomplished through various administration or dosing regimens. Administration of the combination of the invention includes administration of the individual agents of the combination in a single formulation or unit dosage form. Administration of the combination of the invention further includes administration of the individual agents of the combination concurrently or separately and in any order. In some embodiments, the individual agents of the combination may be administered separately or as a fixed combination. In one embodiment, the individual agents of the combination may be administered sequentially by any suitable route. For example, the method of treating cancer according to the invention may comprise (i) administration of the first agent (a) in free or pharmaceutically acceptable salt form and (ii) administration of an agent (b) in free or pharmaceutically acceptable salt form, simultaneously or sequentially in any order, wherein the amounts together are effective in treating cancer, preferably in synergistically effective amounts, e.g., in daily or intermittently dosages corresponding to the amounts described herein. The individual agents of the combination of the invention may be administered separately at different times during the course of therapy or concurrently in divided or single combination forms. Furthermore, the term administering also encompasses the use of a pro-drug of a combination agent that convert in vivo to the combination agent as such. The present invention is therefore to be understood as embracing all such regimens of simultaneous or alternating treatment and the term “administering” is to be interpreted accordingly.

The compounds of the combination of the present invention can be administered intermittently, concurrently or sequentially. In an embodiment, the compounds of the combination of the present invention can be administered in a concurrent dosing regimen.

Repetition of the administration or dosing regimens may be conducted as necessary to achieve the desired reduction or diminution of cancer cells. A “continuous dosing schedule,” as used herein, is an administration or dosing regimen without dose interruptions, e.g., without days off treatment. Repetition of 21 or 28 day treatment cycles without dose interruptions between the treatment cycles is an example of a continuous dosing schedule. In an embodiment, the compounds of the combination of the present invention can be administered in a continuous dosing schedule. In an embodiment, the compounds of the combination of the present invention can be administered concurrently in a continuous dosing schedule.

In one aspect, the invention provides a combination which is synergistic. In some such embodiments, the invention provides a synergistic combination comprising: a. (i) palbociclib, or a pharmaceutically acceptable salt thereof; and (ii) a PD-1 binding antagonist; for use in the treatment of cancer in a subject, wherein component (i) and component (ii) are synergistic; b. (i) palbociclib, or a pharmaceutically acceptable salt thereof; (ii) a PD-1 binding antagonist; and (iii) an OX40 agonist; for use in the treatment of cancer in a subject, wherein component (i) and component (ii); component (i) and component (iii); component (ii) and component (ii); or component (i), component (ii) and component (iii) are synergistic; c. (i) palbociclib, or a pharmaceutically acceptable salt thereof; (ii) a PD-1 binding antagonist; and (ii) a 4-1BB agonist; for use in the treatment of cancer in a subject, wherein component (i) and component (ii); component (i) and component (iii); component (ii) and component (iii); or component (i), component (ii) and component (iii) are synergistic; or d. (i) palbociclib, or a pharmaceutically acceptable salt thereof; (ii) a PD-1 binding antagonist; (iii) an OX40 agonist; and (iv) a 4-1BB agonist; for use in the treatment of cancer in a subject, wherein component (i) and component (ii);

component (i) and component (iii); component (i) and component (iv); component (ii) and component (iii); component (ii) and component (iv); component (iii) and component (iv); component (i) component (ii) and component (iii); component (i), component (ii) and component (iv); component (ii), component (iii) and component (iv); or component (i), component (ii), component (iii) and component (iv) are synergistic.

In another embodiment, the invention provides a synergistic combination comprising: a. (i) palbociclib, or a pharmaceutically acceptable salt thereof; and (ii) a PD-L1 binding antagonist; for use in the treatment of cancer in a subject, wherein component (i) and component (ii) are synergistic; b. (i) palbociclib, or a pharmaceutically acceptable salt thereof; (ii) a PD-L1 binding antagonist; and (iii) an OX40 agonist; for use in the treatment of cancer in a subject, wherein component (i) and component (ii); component (i) and component (iii); component (ii) and component (iii); or component (i), component (ii) and component (iii) are synergistic; c. (i) palbociclib, or a pharmaceutically acceptable salt thereof; (ii) a PD-L1 binding antagonist; and (iii) a 4-1BB agonist; for use in the treatment of cancer in a subject; wherein component (i) and component (ii); component (i) and component (iii); component (ii) and component (iii); or component (i), component (ii) and component (iii) are synergistic; d. (i) palbociclib, or a pharmaceutically acceptable salt thereof; (ii) a PD-L1 binding antagonist; (iii) an OX40 agonist; and (iv) a 4-1BB agonist; for use in the treatment of cancer in a subject, wherein component (i) and component (ii); component (i) and component (iii); component (i) and component (iv); component (ii) and component (iii); component (ii) and component (iv); component (iii) and component (iv); component (i) component (ii) and component (iii); component (i) component (ii) and component (iv); component (ii), component (iii) and component (iv); or component (i), component (ii), component (iii) and component (iv) are synergistic; or e. (i) palbociclib, or a pharmaceutically acceptable salt thereof; (ii) a PD-1 binding antagonist; (iii) a PD-L1 binding antagonist; (iv) an OX40 agonist; and (v) a 4-1BB agonist; for use in the treatment of cancer in a subject, wherein component (i) and component (ii); component (i) and component (iii); component (i) and component (iv);

component (i) and component (v); component (ii) and component (iii); component (ii) and component (iv); component (ii) and component (v); component (iii) and component (iv); component (iii) and component (v); component (iii) and component (v); component (iv) and component (v); component (i), component (ii) and component (iii); component (i), component (ii) and component (iv); component (i), component (ii) and component (v);

component (ii), component (iii) and component (iv); component (ii), component (iii) and component (v); component (ii), component (iv) and component (v); component (iii), component (vi) and component (v); component (i), component (ii), component (iii) and component (iv); component (i), component (ii), component (iii) and component (v); component (i), component (iii), component (iv) and component (v); or component (ii), component (iii), component (iv) and component (v) are synergistic.

In yet another embodiment, the invention provides a synergistic combination comprising: a. (i) 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)- one, or a pharmaceutically acceptable salt thereof; and (ii) a PD-1 binding antagonist; for use in the treatment of cancer in a subject, wherein component (i) and component (ii) are synergistic; b. (i) 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)- one, or a pharmaceutically acceptable salt thereof; (ii) a PD-1 binding antagonist; and (iii) an OX40 agonist; for use in the treatment of cancer in a subject, wherein component (i) and component (ii); component (i) and component (iii); component (ii) and component (iii); or component (i), component (ii) and component (iii) are synergistic; c. (i) 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof; (ii) a PD-1 binding antagonist; and (iii) a 4-1BB agonist; for use in the treatment of cancer in a subject, wherein component (i) and component (ii); component (i) and component (iii); component (ii) and component (iii); or component (i), component (ii) and component (iii) are synergistic; d. (i) 6-(difluoromethyl)-8-((1R,2R)-2-hydroxy-2-methylcyclopentyl)-2-(1-(methylsulfonyl)piperidin-4-ylamino)pyrido[2,3-d]pyrim idin-7(8H)-one, or a pharmaceutically acceptable salt thereof; (ii) a PD-1 binding antagonist; (iii) an OX40 agonist; and (iv) a 4-1BB agonist; for use in the treatment of cancer in a subject, wherein component (i) and component (ii); component (i) and component (iii); component (i) and component (iv); component (ii) and component (iii); component (ii) and component (iv);

component (iii) and component (iv); component (i), component (ii) and component (iii); component (i), component (ii) and component (iv); component (ii), component (iii) and component (iv); or component (i), component (ii), component (iii) and component (iv) are synergistic. In a further embodiment, the invention is related to synergistic combination comprising: a. (i) 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)- one, or a pharmaceutically acceptable salt thereof; and (ii) a PD-L1 binding antagonist; for use in the treatment of cancer in a subject, wherein component (i) and component (ii) are synergistic; b. (i) 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)- one, or a pharmaceutically acceptable salt thereof; (ii) a PD-L1 binding antagonist; and (iii) an OX40 agonist; for use in the treatment of cancer in a subject, wherein component (i) and component (ii); component (i) and component (iii); component (ii) and component (iii); or component (i), component (ii) and component (iii) are synergistic; c. (i) 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof; (ii) a PD-L1 binding antagonist; and (iii) a 4-1 BB agonist; for use in the treatment of cancer in a subject, wherein component (i) and component (ii); component (i) and component (iii); component (ii) and component (iii); or component (i), component (ii) and component (iii) are synergistic; d. (i) 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof; (ii) a PD-L1 binding antagonist; (iii) an OX40 agonist; and (iv) a 4-1BB agonist; for use in the treatment of cancer in a subject, wherein component (i) and component (ii); component (i) and component (iii); component (i) and component (iv); component (ii) and component (iii); component (ii) and component (iv); component (iii) and component (iv); component (i), component (ii) and component (iii); component (i), component (ii) and component (iv); component (ii), component (iii) and component (iv); or component (i), component (ii), component (iii) and component (iv) are synergistic; or e. (i) 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof; (ii) a PD-1 binding antagonist, (iii) a PD-L1 binding antagonist, (iv) an OX40 agonist, and (v) an anti-4-1BB antibody; for use in the treatment of cancer in a subject, wherein component (i) and component (ii); component (i) and component (iii); component (i) and component (iv); component (i) and component (v); component (ii) and component (iii); component (ii) and component (iv); component (ii) and component (v); component (iii) and component (iv); component (iii) and component (v); component (iii) and component (v); component (iv) and component (v);

component (i), component (ii) and component (iii); component (i), component (ii) and component (iv); component (i), component (ii) and component (v); component (ii), component (iii) and component (iv); component (ii), component (iii) and component (v); component (ii), component (iv) and component (v); component (iii), component (iv) and component (v); component (i), component (ii), component (iii) and component (iv);

component (i), component (ii), component (iii) and component (v); component (i), component (iii), component (iv) and component (v); or component (ii), component (iii), component (iv) and component (v) are synergistic.

In one embodiment, the present invention provides a combination comprising: a. palbociclib, or a pharmaceutically acceptable salt thereof, and a PD-1 binding antagonist; b. palbociclib, or a pharmaceutically acceptable salt thereof, a PD-1 binding antagonist, and an OX40 agonist; c. palbociclib, or a pharmaceutically acceptable salt thereof, a PD-1 binding antagonist, and a 4-1BB agonist; or d. palbociclib, or a pharmaceutically acceptable salt thereof, a PD-1 binding antagonist, an OX40 agonist and a 4-1BB agonist, for use in the treatment of cancer in a subject.

In another embodiment, the present invention provides a combination comprising: a. palbociclib, or a pharmaceutically acceptable salt thereof, and a PD-L1 binding antagonist; b. palbociclib, or a pharmaceutically acceptable salt thereof, a PD-L1 binding antagonist, and a 4-1BB agonist; c. palbociclib, or a pharmaceutically acceptable salt thereof, a PD-L1 binding antagonist, and an OX40 agonist; d. palbociclib, or a pharmaceutically acceptable salt thereof, a PD-L1 binding antagonist, a 4-1BB agonist; or e. palbociclib, or a pharmaceutically acceptable salt thereof, a PD-1 binding antagonist, a PD-L1 binding antagonist, an OX40 agonist and a 4-1BB agonist, for use in the treatment of cancer in a subject.

In yet another embodiment, the present invention provides a combination comprising: a. 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof, and a PD-1 binding antagonist; b. 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof, a PD-1 binding antagonist, and an OX40 agonist; c. 6-(difluoromethyl)-8-((1R,2R)-2-hydroxy-2-methylcyclopentyl)-2-(1-(methylsulfonyl)piperidin-4-ylam ino)pyrido[2,3-d]pyrim idin-7(8H)-one, or a pharmaceutically acceptable salt thereof, a PD-1 binding antagonist, and a 4-1BB agonist; or d. 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof, a PD-1 binding antagonist, an OX40 agonist, and a 4-1BB agonist, for use in the treatment of cancer in a subject.

In one embodiment, the present invention provides a combination comprising: a. 6-(difluoromethyl)-8-((1R,2R)-2-hydroxy-2-methylcyclopentyl)-2-(1-(methylsulfonyl)-piperidin-4-ylamino)pyrido[2,3-d]pyrimidin-7(8H)-one, or a pharmaceutically acceptable salt thereof, and a PD-L1 binding antagonist; b. 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof, a PD-L1 binding antagonist, and an OX40 agonist; c. 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof, a PD-L1 binding antagonist, and a 4-1BB agonist; d. 6-(difluoromethyl)-8-((1R,2R)-2-hydroxy-2-methylcyclopentyl)-2-(1-(methyl-sulfonyl)piperidin-4-ylamino)pyrido[2,3-d]pyrimidin-7(8H)-one, or a pharmaceutically acceptable salt thereof, a PD-1 binding antagonist, a PD-L1 binding antagonist, an OX40 agonist and a 4-1BB agonist; or e. 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof, a PD-1 binding antagonist, a PD-L1 binding antagonist, an OX40 agonist and a 4-1BB agonist, for use in the treatment of cancer in a subject.

In a particular embodiment of each of the foregoing, the invention provides a combination wherein the PD-1 binding antagonist is an anti-PD-1 antibody; the PD-L1 binding antagonist is an anti-PD-L1 antibody; the OX40 agonist is an anti-OX40 antibody; and/or the 4-1BB agonist is an anti-4-1BB antibody.

In some embodiments of the each of the foregoing, the subject is intended to include animals. Examples of subjects include mammals, e.g., humans, cows, sheep, cats, dogs, horses, primates, rabbits, and rodents (e.g., mice and rats), and transgenic non-human animals. In a particular embodiment, the subject is a human, e.g., a human suffering from, at risk of suffering from, or potentially capable of suffering from cancer.

In further embodiments of each of the foregoing, the cancer is a solid tumor. In some embodiments, the cancer is a hematologic cancer. In some embodiments of the each of the foregoing, the cancer is selected from the group consisting of brain cancer, head/neck cancer (including squamous cell carcinoma of the head and neck (SCCHN)), prostate cancer, ovarian cancer, bladder cancer (including urothelial carcinoma, also known as transitional cell carcinoma (TCC)), lung cancer (including squamous cell carcinoma, small cell lung cancer (SCLC), and non-small cell lung cancer (NSCLC)), breast cancer, bone cancer, colorectal cancer, kidney cancer, liver cancer (including hepatocellular carcinoma (HCC)), stomach cancer, pancreatic cancer, esophageal cancer , cervical cancer, sarcoma, skin cancer (including melanoma and Merkel cell carcinoma (MCC)), multiple myeloma, mesothelioma, malignant rhabdoid tumors, neuroblastoma, diffuse intrinsic pontine glioma (DIPG), carcinoma, lymphoma, diffuse large B-cell lymphoma (DLBCL), primary mediastinal B-cell lymphoma (PMBCL), follicular lymphoma, acute lymphoblastic leukemia (ALL), acute myeloid leukemia (AML), chronic lymphocytic leukemia (CLL), chronic myeloid leukemia (CML), follicular lymphoma, Hodgkin's lymphoma (HL), classical Hodgkin lymphoma (cHL), mantle cell lymphoma (MCL), multiple myeloma (MM), myeloid cell leukemia-1 protein (Mcl-1), myelodysplastic syndrome (MDS), non-Hodgkin's lymphoma (NHL), small lymphocytic lymphoma (SLL), and SWI/SNF-mutant cancer.

### IX. Kits

In one aspect, the invention provides a kit comprising: a. (i) a pharmaceutical composition comprising a CDK inhibitor and a pharmaceutically acceptable carrier; (ii) a pharmaceutical composition comprising a PD-1 binding antagonist and a pharmaceutically acceptable carrier; b. (i) a pharmaceutical composition comprising a CDK inhibitor and a pharmaceutically acceptable carrier; (ii) a pharmaceutical composition comprising a PD-1 binding antagonist and a pharmaceutically acceptable carrier; (iii) a pharmaceutical composition comprising an OX40 agonist and a pharmaceutically acceptable carrier; c. (i) a pharmaceutical composition comprising a CDK inhibitor and a pharmaceutically acceptable carrier; (ii) a pharmaceutical composition comprising a PD-1 binding antagonist and a pharmaceutically acceptable carrier; (iii) a pharmaceutical composition comprising a 4-1BB agonist and a pharmaceutically acceptable carrier; or d. (i) a pharmaceutical composition comprising a CDK inhibitor and a pharmaceutically acceptable carrier; (ii) a pharmaceutical composition comprising PD-1 binding antagonist and a pharmaceutically acceptable carrier; (iii) a pharmaceutical composition comprising an OX40 agonist and a pharmaceutically acceptable carrier; (iv) a pharmaceutical composition comprising a 4-1BB agonist and a pharmaceutically acceptable carrier; and instructions for dosing of the pharmaceutical compositions for the treatment of cancer. In one embodiment, the PD-1 binding antagonist is an anti-PD-1 antibody; the OX40 agonist is an anti-OX40 antibody; and/or the 4-1 BB agonist is an anti-4-1 BB antibody.

In one aspect of the invention, a kit is provided comprising: a. (i) a pharmaceutical composition comprising a CDK inhibitor and a pharmaceutically acceptable carrier; (ii) a pharmaceutical composition comprising a PD-L1 binding antagonist and a pharmaceutically acceptable carrier; b. (i) a pharmaceutical composition comprising a CDK inhibitor and a pharmaceutically acceptable carrier; (ii) a pharmaceutical composition comprising a PD-L1 binding antagonist and a pharmaceutically acceptable carrier; (iii) a pharmaceutical composition comprising an OX40 agonist and a pharmaceutically acceptable carrier; c. (i) a pharmaceutical composition comprising a CDK inhibitor and a pharmaceutically acceptable carrier; (ii) a pharmaceutical composition comprising a PD-L1 binding antagonist and a pharmaceutically acceptable carrier; (iii) a pharmaceutical composition comprising a 4-1BB agonist and a pharmaceutically acceptable carrier; d. (i) a pharmaceutical composition comprising a CDK inhibitor and a pharmaceutically acceptable carrier; (ii) a pharmaceutical composition comprising PD-L1 binding antagonist and a pharmaceutically acceptable carrier; (iii) a pharmaceutical composition comprising an OX40 agonist and a pharmaceutically acceptable carrier; (iv) a pharmaceutical composition comprising a 4-1BB agonist and a pharmaceutically acceptable carrier; or e. (i) a pharmaceutical composition comprising a CDK inhibitor and a pharmaceutically acceptable carrier; (ii) a pharmaceutical composition comprising a PD-1 binding antagonist and a pharmaceutically acceptable carrier; (iii) a pharmaceutical composition comprising PD-L1 binding antagonist and a pharmaceutically acceptable carrier; (iv) a pharmaceutical composition comprising an OX40 agonist and a pharmaceutically acceptable carrier; and (v) a pharmaceutical composition comprising a 4-1BB agonist and a pharmaceutically acceptable carrier.

In some embodiments, the kit further comprises package insert comprising instructions for using the CDK inhibitor in conjunction with PD-1 axis binding antagonist (e.g., anti-PD-1 or anti-PD-L1 antibody), the OX40 agonist (e.g., anti-human OX40 agonist antibody) and/or 4-BB agonist (e.g., anti-human 4-1BB agonist antibody) treat or delay progression of cancer in an individual or to enhance immune function of a subject having cancer. In further embodiment, any of the CDK inhibitors, PD-1 axis binding antagonists, OX40 agonist and/or 4-1BB agonists described herein may be included in the kits.

For example, in some embodiments, the CDK inhibitor is a CDK4/6 inhibitor. In some such embodiments, the CDK4/6 inhibitor is palbociclib, or a pharmaceutically acceptable salt thereof. In another embodiment, the CDK inhibitor is a CDK2/4/6 inhibitor. In a particular embodiment, the CD2/4/6 inhibitor is 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof. In specific embodiments, the PD-L1 binding antagonist is an anti-PD-L1 antibody. In yet another specific embodiment, the OX40 agonist is an anti-OX40 antibody; and/or the 4-1 BB agonist is an anti-4-1 BB antibody.

In some embodiments, the PD-1 axis binding antagonist, the OX40 binding agonist (e.g., anti-human OX40 agonist antibody), and/or the 4-1 BB agonist are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The kit may further include other materials desirable from a commercial and user standpoint, including other buffers, diluents, filters, needles, syringes, and package inserts with instructions for use. In some embodiments, the kit further includes one or more of another agent (e.g., a chemotherapeutic agent, and anti-neoplastic agent). Suitable containers for the one or more agent include, for example, bottles, vials, bags and syringes.

The specification is sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: CDK4/6 Inhibitor, Palbociclib Synergizes with PD-L1 Based Immune Checkpoint Blockade in the MC38 Syngeneic Mouse Tumor Model

### Overview

Palbociclib was evaluated in the MC38 syngeneic mouse tumor model in combination with antibodies targeting PD-L1, 4-1BB and OX40 to assess efficacy on primary tumor growth and survival. Palbociclib in combination with immune checkpoint blockade agents led to significant tumor growth inhibition (p=0.0000001).

### Materials and Methods

MC38 cells were obtained from American Type Culture Collection (ATCC) and cultured in Roswell Park Memorial Institute (RPMI1640) supplemented with 10% fetal bovine serum (FBS). All cells were maintained in a humidified incubator at 37° C. with 5% carbon dioxide (CO₂). Female C57/BL6 mice were obtained from Jackson Laboratories at 8 weeks of age. To generate the syngeneic model, 0.5 million MC38 tumor cells were subcutaneously implanted into the right flank of female C57/BL6 mice. Tumor bearing mice were randomized into four treatment groups based on average tumor sizes of approximately 70 mm³ per group, on Day 9 post tumor cell implantation. Study groups included vehicle, 15 mg/kg palbociclib twice daily by oral gavage, combination of 10 mg/kg avelumab (anti-PD-L1 antibody, PF-06834635) administered by intraperitoneal (IP) injection, 5mg/kg anti-OX40 antibody (PF-07201252) administered by IP injection and 10 mg/kg with anti-4-1BB antibody (PF-07218859) administered at 3 mg/kg by IP injection, and combination of 15 mg/kg palbociclib twice daily by oral gavage with 10 mg/kg avelumab (anti-PD-L1 antibody, PF-06834635) administered by intraperitoneal (IP) injection, 5 mg/kg anti-OX40 antibody (PF-07201252) administered by IP injection and 10 mg/kg with anti-4-1BB antibody (PF-07218859) administered at 3 mg/kg by IP injection. All antibodies were administered as three doses every three days after the study initiation. All antibody formulations are phosphate buffered saline based while palbociclib was administered in a 0.5% methocel/Tween suspension. The treatment groups and dose regimen information are summarized in Table 8.

TABLE 8

| | | Animals/ | | |
|---|---|---|---|---|
| Group | Drug | group | Route | Regimen |
| 1 | vehicle | 10 | PO | BID |
| | | | | continuously |
| 2 | palbociclib 15 mg/kg | 10 | PO | BID |
| | | | | continuously |
| 3 | PF-06834635 10 | 10 | IP + IP + | QD3; 3 doses + |
| | mg/kg + | | IP | QD3; 3 doses + |
| | PF-07201252 5 mg/kg + | | | QD3; 3 doses |
| | PF-07218859 3 mg/kg | | | |
| 4 | PF-06834635 10 | 10 | IP + IP + | QD3; 3 doses + |
| | mg/kg + | | IP + PO | QD3; 3 doses + |
| | PF-07201252 5 mg/kg + | | | QD3; 3 doses + |
| | PF-07218859 3 mg/kg + | | | BID |
| | palbociclib 15 mg/kg | | | continuously |

BID = twice daily;
PO = oral dosing;
QD3 = 1 dose every 3 days

Tumor volumes were measured three times a week. Tumor volume was calculated based on two-dimensional caliper measurement with cubic millimeter volume calculated using the formula (length×width2)×0.5. Mice were sacrificed when the tumor volumes reached 2000 mm³, which was the survival endpoint for this study. Survival curves were plotted using GraphPad Prism 7 software. Statistical significance determined using the Holm-Sidak method, with alpha=0.05.

### Results

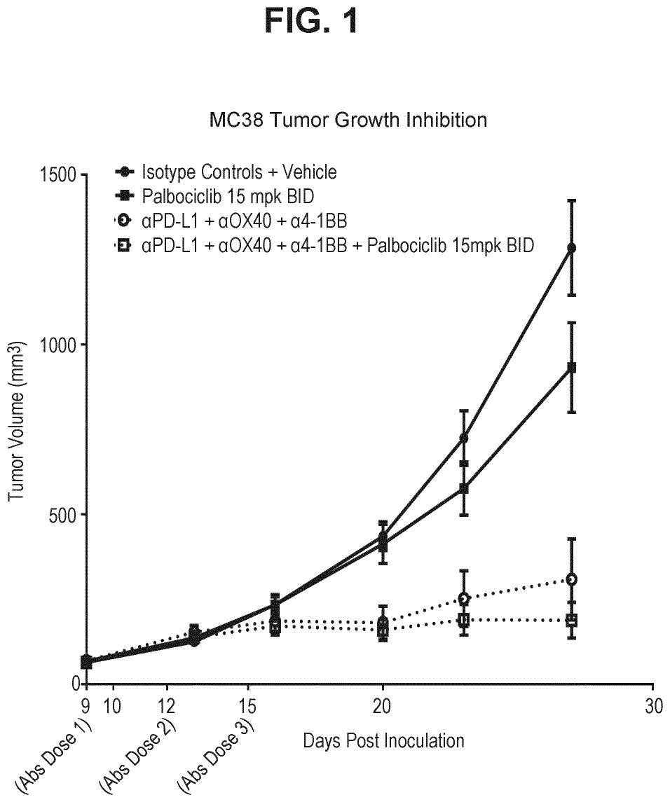

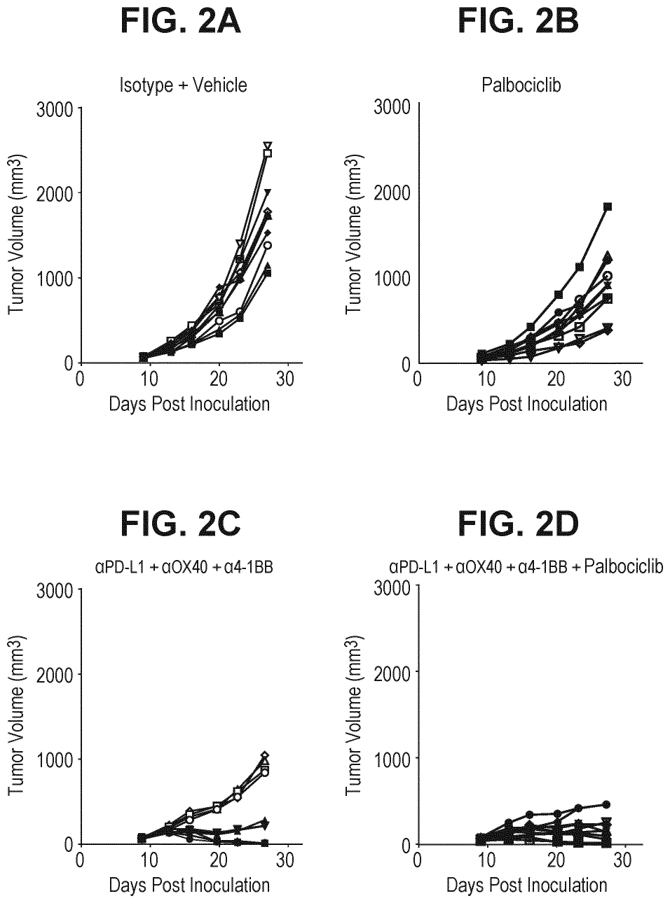

On Day 27 post-treatment initiation, tumor growth results show that treatment with the CDK4/6 inhibitor palbociclib monotherapy did not significantly inhibit tumor growth in the MC38 xenograft tumor model. However, palbociclib treatment in combination with avelumab+anti-OX40 antibody+anti-4-1 BB antibody (p=0.0000008) showed a trend to a combinatorial effect, with increase in tumor growth inhibition. These data are summarized as mean tumor volume in FIG. 1, individual tumor volumes in FIG. 2 and absolute values in Table 9.

TABLE 9

| | | P values (vs on | TGI % |
|---|---|---|---|
| Group | Agent | vehicle) day 27 | on day 27 |
| 1 | vehicle | N/A | 0 |
| 2 | palbociclib 15 mg/kg | 0.08 | 29 |
| 3 | PF-06834635 10 mg/kg + | 0.000008 | 80 |
| | PF-07201252 5 mg/kg + | | |
| | PF-07218859 3 mg/kg | | |
| 4 | PF-06834635 10 mg/kg + | p = 0.0000008 | 90 |
| | PF-07201252 5 mg/kg + | | |
| | PF-07218859 3 mg/kg + | | |
| | palbociclib 15 mg/kg | | |

### Conclusions

Combination of palbociclib with checkpoint blockade antibodies led to greater tumor growth inhibition and significant improvement in survival relative to palbociclib monotherapy, or the combination of anti-4-1BB antibody, anti-OX40 antibody and avelumab alone in the MC38 syngeneic tumor model.

### Example 2: CDK2/4/6 Inhibitor (PF-068736000) Synergizes with PD-L1 Based Immune Checkpoint Blockade in the MC38 Syngeneic Mouse Tumor Model

### Overview

PF-06873600 was evaluated in the MC38 syngeneic mouse tumor model in combination with antibodies targeting PD-L1, 4-1BB and OX40 to assess efficacy on primary tumor growth and survival. PF-06873600 in combination with immune checkpoint blockade agents led to significant tumor growth inhibition with the most pronounced effect the combination of PF-06873600 combined with PD-L1 and OX40 targeting antibodies (p=0.0000001).

### Materials and Methods

MC38 cells were obtained from American Type Culture Collection (ATCC) and cultured in Roswell Park Memorial Institute (RPMI1640) supplemented with 10% fetal bovine serum (FBS). All cells were maintained in a humidified incubator at 37° C. with 5% carbon dioxide (CO2). Female C57/BL6 mice were obtained from Jackson Laboratories at 8 weeks of age. To generate the syngeneic model, 0.5 million MC38 tumor cells were subcutaneously implanted into the right flank of female C57/BL6 mice. Tumor bearing mice were randomized into nine treatment groups based on average tumor sizes of approximately 70 mm³ per group, on Day 9 post tumor cell implantation. Study groups included vehicle, 30 mg/kg PF-06873600 (CDK 2/4/6 inhibitor) twice daily by oral gavage, avelumab (anti-PD-L1 antibody, PF-06834635) administered by intraperitoneal (IP) injection, 10 mg/kg, combination of avelumab administered at 10 mg/kg with anti-OX40 antibody (PF-07201252) administered at 5 mg/kg by IP injection, combination of anti-PD-L1 antibody administered at 10 mg/kg with anti-4-1BB antibody (PF-07218859) administered at 3 mg/kg by IP injection, combination of anti-PD-L1 antibody as described above with PF-06873600 administered at 30mg/kg twice daily by oral gavage, combination of anti-PD-L1 antibody and anti-OX40 antibody as described above with PF-06873600 administered at 30 mg/kg twice daily by oral gavage, combination of anti-PD-L1 antibody with anti-4-1BB antibody as described above with PF-06873600 administered at 30mg/kg twice daily by oral gavage and combination of anti-PD-L1 antibody, anti-OX40 antibody and anti-41-BB antibody as described above with PF-06873600 administered at 30 mg/kg twice daily by oral gavage. All antibodies were administered as three doses; one every three days after the study initiation. All antibody formulations are phosphate buffered saline based while PF-06873600 was administered in a 0.5% methocel/Tween suspension. The treatment groups and dose regimen information are summarized in Table 10.

TABLE 10

| | | Animals/ | | |
|---|---|---|---|---|
| Group | Drug | group | Route | Regimen |
| 1 | vehicle | 10 | PO | BID |
| | | | | continuously |
| 2 | PF-06873600 30 mg/kg | 10 | PO | BID |
| | | | | continuously |
| 3 | PF-06834635 10 mg/kg | 10 | IP | QD3; 3 doses |
| 4 | PF-06834635 10 mg/kg + | 10 | IP + IP | QD3; 3 doses + |
| | PF-07201252 5 mg/kg | | | QD3; 3 doses |
| 5 | PF-06834635 10 mg/kg + | 10 | IP + IP | QD3; 3 doses + |
| | PF-07218859 3 mg/kg | | | QD3; 3 doses |
| 6 | PF-06834635 10 mg/kg + | 10 | IP + PO | QD3; 3 doses + |
| | PF-06873600 30 mg/kg | | | BID |
| | | | | continuously |
| 7 | PF-06834635 10 mg/kg + | 10 | IP + IP + | QD3; 3 doses + |
| | PF-07201252 5 mg/kg + | | PO | QD3; 3 doses + |
| | PF-06873600 30 mg/kg | | | BID |
| | | | | continuously |
| 8 | PF-06834635 10 mg/kg + | 10 | IP + IP + | QD3; 3 doses + |
| | PF-07218859 3 mg/kg + | | PO | QD3; 3 doses + |
| | PF-06873600 30 mg/kg | | | BID |
| | | | | continuously |
| 9 | PF-06834635 10 mg/kg + | 10 | IP + IP + | QD3; 3 doses + |
| | PF-07201252 5 mg/kg + | | IP | QD3; 3 doses + |
| | PF-07218859 3 mg/kg | | | QD3; 3 doses |
| 10 | PF-06834635 10 mg/kg + | 10 | IP + IP + | QD3; 3 doses + |
| | PF-07201252 5 mg/kg + | | IP + PO | QD3; 3 doses + |
| | PF-07218859 3 mg/kg + | | | QD3; 3 doses + |
| | PF-06873600 30 mg/kg | | | BID |
| | | | | continuously |

BID = twice daily;
PO = oral dosing;
QD3 = 1 dose every 3 days

Tumor volumes were measured three times a week. Tumor volume was calculated based on two-dimensional caliper measurement with cubic millimeter volume calculated using the formula (length×width2)×0.5. Mice were sacrificed when the tumor volumes reached 2000 mm³, which was the survival endpoint for this study. Survival curves were plotted using GraphPad Prism 7 software. Statistical significance determined using the Holm-Sidak method, with alpha=0.05.

### Results

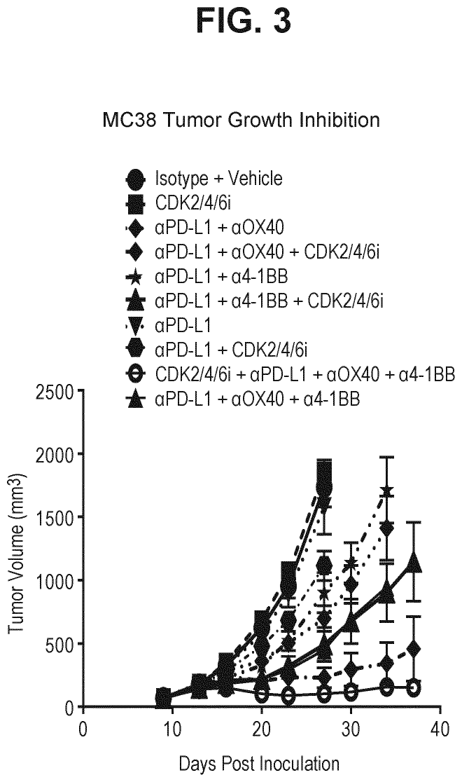

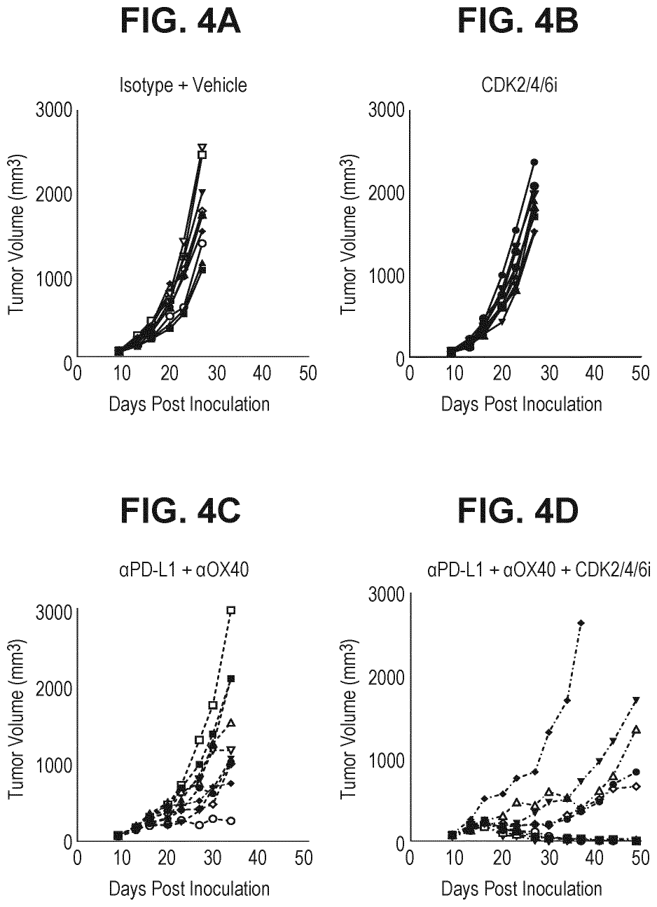

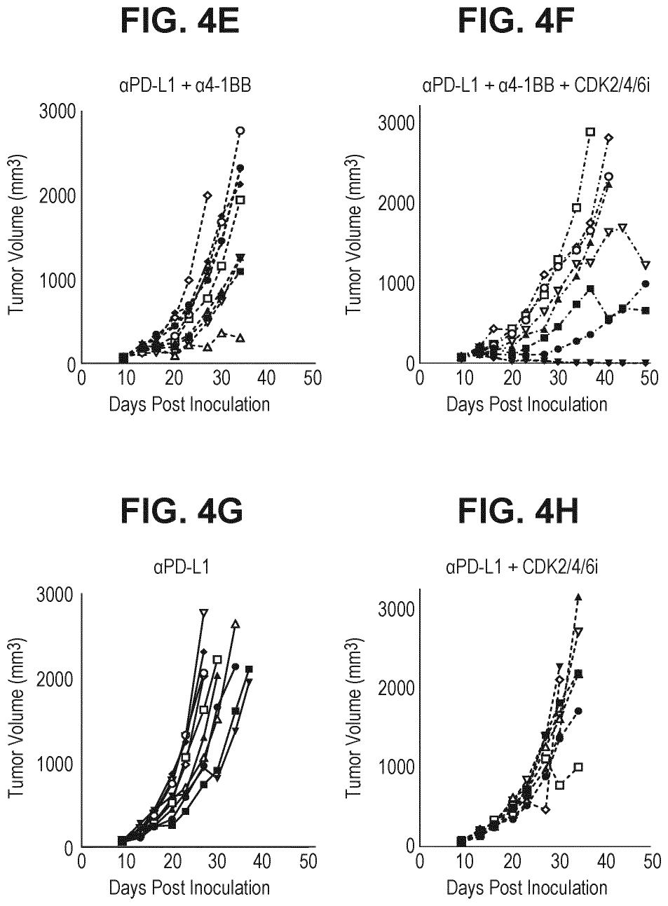

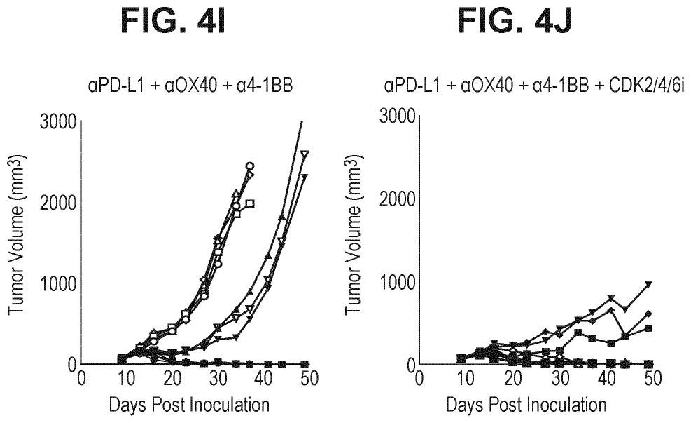

On Day 27 post-treatment initiation, tumor growth results show that treatment with the either avelumab or the CDK2/4/6 inhibitor PF06873600 monotherapy did not significantly inhibit tumor growth in the MC38 xenograft tumor model. However, PF06873600 treatment in combination with avelumab (p=0.08), avelumab+anti-OX40 antibody (p=0.0000001), avelumab+anti-4-1 BB antibody (p=0.00002) or avelumab+anti-OX40 antibody +anti-4-1 BB antibody (p=0.000000009) showed a trend to a combinatorial effect, with increase in tumor growth inhibition. These data are summarized as mean tumor volume in FIG. 3, individual tumor volumes in FIG. 4 and absolute values in Table 11.

TABLE 11

| | | P values (vs | TGI % | CR % |
|---|---|---|---|---|
| | | vehicle) on | on | on |
| Group | Agent | day 27 | day 27 | day 27 |
| 1 | vehicle | N/A | 0 | 0 |
| 2 | PF-06873600 30 mg/kg | 0.52 | −7 | 0 |
| 3 | PF-06834635 10 mg/kg | 0.57 | 9 | 0 |
| 4 | PF-06834635 10 mg/kg + | 0.00003 | 62 | 0 |
| | PF-07201252 5 mg/kg | | | |
| 5 | PF-06834635 10 mg/kg + | 0.0016 | 50 | 0 |
| | PF-07218859 3 mg/kg | | | |
| 6 | PF-06834635 10 mg/kg + | 0.008 | 37 | 0 |
| | PF-06873600 30 mg/kg | | | |
| 7 | PF-06834635 10 mg/kg + | 0.0000001 | 90 | 50 |
| | PF-07201252 5 mg/kg + | | | |
| | PF-06873600 30 mg/kg | | | |
| 8 | PF-06834635 10 mg/kg + | 0.00002 | 75 | 22 |
| | PF-07218859 3 mg/kg + | | | |
| | PF-06873600 30 mg/kg | | | |
| 9 | PF-06834635 10 mg/kg + | 0.000008 | 77 | 30 |
| | PF-07201252 5 mg/kg + | | | |
| | PF-07218859 3 mg/kg | | | |
| 10 | PF-06834635 10 mg/kg + | 0.000000009 | 98 | 70 |
| | PF-07201252 5 mg/kg + | | | |
| | PF-07218859 3 mg/kg + | | | |
| | PF-06873600 30 mg/kg | | | |

### Conclusions

Combination of the CDK2/4/6 inhibitor (PF-06873600) with checkpoint blockade antibodies led to greater tumor growth inhibition and significant improvement in survival relative to avelumab monotherapy, PF-06873600 monotherapy, or the combination of anti-4-1 BB antibody or anti-OX40 antibody with avelumab alone in the MC38 syngeneic tumor model.

### Example 3: The CDK4/6 Inhibitor, Palbociclib Synergizes with OX40/4-1BB Immune Checkpoint Modulators in the MC38 Syngeneic Mouse Tumor Model

### Overview Palbociclib will be evaluated in the MC38 syngeneic mouse tumor model in combination with antibodies targeting 4-1BB and OX40 to assess efficacy on primary tumor growth and survival.

### Materials and Methods

MC38 cells will be obtained from American Type Culture Collection (ATCC) and cultured in Roswell Park Memorial Institute (RPMI1640) supplemented with 10% fetal bovine serum (FBS). All cells will be maintained in a humidified incubator at 37° C. with 5% carbon dioxide (CO₂). Female C57/BL6 mice will be obtained from Jackson Laboratories at 8 weeks of age. To generate the syngeneic model, 0.5 million MC38 tumor cells will be subcutaneously implanted into the right flank of female C57/BL6 mice. Tumor bearing mice will be randomized into six treatment groups based on average tumor sizes of approximately 70 mm³ per group, on Day 9 post tumor cell implantation. Study groups included vehicle, 15 mg/kg palbociclib twice daily by oral gavage, anti-OX40 antibody (PF-07201252) administered at 5 mg/kg by intraperitoneal (IP) injection, anti-4-1BB antibody (PF-07218859) administered at 3 mg/kg by IP injection, combination of 15 mg/kg palbociclib twice daily by oral gavage and anti-OX40 (PF-07201252) administered at 5 mg/kg by intraperitoneal (IP) injection, combination of 15 mg/kg palbociclib twice daily by oral gavage and anti-4-1BB antibody (PF-07218859) administered at 3 mg/kg by IP injection, combination of anti-OX40 antibody (PF-07201252) administered at 5 mg/kg by intraperitoneal (IP) injection and anti-4-1BB antibody (PF-07218859) administered at 3 mg/kg by IP injection and combination of PF-06873600 twice daily by oral gavage with anti-OX40 antibody (PF-07201252) administered at 5 mg/kg by intraperitoneal (IP) injection and anti-4-1BB antibody (PF-07218859) administered at 3 mg/kg by IP injection every three days for three doses. All antibodies will be administered as three doses; one every three days after the study initiation. All antibody formulations are phosphate buffered saline based while PF-06873600 will be administered in a 0.5% methocel/Tween suspension. The treatment groups and dose regimen information are summarized in Table 12.

TABLE 12

| | | Animals/ | | |
|---|---|---|---|---|
| Group | Drug | group | Route | Regimen |
| 1 | vehicle | 10 | PO | BID |
| | | | | continuously |
| 2 | palbociclib 15 mg/kg | 10 | PO | BID |
| | | | | continuously |
| 3 | PF-07201252 5 mg/kg | 10 | IP | QD3; 3 doses |
| 4 | PF-07218859 3 mg/kg | 10 | IP | QD3; 3 doses |
| 5 | PF-07201252 5 mg/kg | 10 | IP + PO | QD3; 3 doses + |
| | palbociclib 15 mg/kg | | | BID |
| | | | | continuously |
| 6 | PF-07218859 3 mg/kg | 10 | IP + PO | QD3; 3 doses + |
| | P palbociclib 15 mg/kg | | | BID |
| | | | | continuously |
| 7 | PF-07201252 5 mg/kg + | 10 | IP + IP | QD3; 3 doses + |
| | PF-07218859 3 mg/kg | | | QD3; 3 doses |
| 8 | PF-07201252 5 mg/kg + | 10 | IP + IP + | QD3; 3 doses + |
| | PF-07218859 3 mg/kg + | | PO | QD3; 3 doses + |
| | palbociclib 15 mg/kg | | | BID |
| | | | | continuously |

BID = twice daily;
PO = oral dosing;
QD3 = 1 dose every 3 days

Tumor volumes will be measured three times a week. Tumor volume will be calculated based on two-dimensional caliper measurement with cubic millimeter volume calculated using the formula (length×width2)×0.5. Mice will be sacrificed when the tumor volumes reached 2000 mm³, which is the survival endpoint for this study. Survival curves will be plotted using GraphPad Prism 7 software and statistical significance determined using the Holm-Sidak method, with alpha=0.05.

### Example 4: CDK4/6 Inhibitor Palbociclib Synergizes with PD-L1 Based Immune Checkpoint Blockade in the MC38 Syngeneic Mouse Tumor Model

### Overview

Palbociclib will be evaluated in the MC38 syngeneic mouse tumor model in combination with antibodies targeting PD-L1, 4-1BB and OX40 to assess efficacy on primary tumor growth and survival.

### Materials and Methods

MC38 cells will be obtained from American Type Culture Collection (ATCC) and cultured in Roswell Park Memorial Institute (RPMI1640) supplemented with 10% fetal bovine serum (FBS). All cells will be maintained in a humidified incubator at 37° C. with 5% carbon dioxide (CO₂). Female C57/BL6 mice will be obtained from Jackson Laboratories at 8 weeks of age. To generate the syngeneic model, 0.5 million MC38 tumor cells will be subcutaneously implanted into the right flank of female C57/BL6 mice. Tumor bearing mice will be randomized into eight treatment groups based on average tumor sizes of approximately 70 mm³ per group, on Day 9 post tumor cell implantation. Study groups will include vehicle, 3015 mg/kg palbociclib twice daily by oral gavage, avelumab (anti-PD-L1 antibody, PF-06834635) administered by intraperitoneal (IP) injection, 10 mg/kg, combination of avelumab administered at 10 mg/kg with anti-OX40 antibody (PF-07201252) administered at 5mg/kg by IP injection, combination of anti-PD-L1 administered at 10 mg/kg with anti-4-1BB antibody (PF-07218859) administered at 3 mg/kg by IP injection, combination of anti-PD-L1 antibody as described above with palbociclib administered at 15 mg/kg twice daily by oral gavage, combination of anti-PD-L1 antibody and anti-OX40 antibody as described above with palbociclib administered at 15 mg/kg twice daily by oral gavage, and combination of anti-PD-L1 antibody with anti-4-1BB antibody as described above with palbociclib administered at 15 mg/kg twice daily by oral gavage. All antibodies will be administered as three doses every three days after the study initiation. All antibody formulations are phosphate buffered saline based while palbociclib is administered in a 0.5% methocel/Tween suspension. The treatment groups and dose regimen information are summarized in Table 13.

TABLE 13

| | | Animals/ | | |
|---|---|---|---|---|
| Group | Drug | group | Route | Regimen |
| 1 | vehicle | 10 | PO | BID |
| | | | | continuously |
| 2 | palbociclib 15 mg/kg | 10 | PO | BID |
| | | | | continuously |
| 3 | PF-06834635 10 mg/kg | 10 | IP | QD3; 3 doses |
| 4 | PF-06834635 10 mg/kg + | 10 | IP + IP | QD3; 3 doses+ |
| | PF-07201252 5 mg/kg | | | QD3; 3 doses |
| 5 | PF-06834635 10 mg/kg + | 10 | IP + IP | QD3; 3 doses + |
| | PF-07218859 3 mg/kg | | | QD3; 3 doses |
| 6 | PF-06834635 10 mg/kg + | 10 | IP + PO | QD3; 3 doses + |
| | palbociclib 15 mg/kg | | | BID |
| | | | | continuously |
| 7 | PF-06834635 10 mg/kg + | 10 | IP + IP + | QD3; 3 doses + |
| | PF-07201252 5 mg/kg + | | PO | QD3; 3 doses + |
| | palbociclib 15 mg/kg | | | BID |
| | | | | continuously |
| 8 | PF-06834635 10 mg/kg + | 10 | IP + IP + | QD3; 3 doses + |
| | PF-07218859 3 mg/kg + | | PO | QD3; 3 doses + |
| | palbociclib 15 mg/kg | | | BID |
| | | | | continuously |

BID = twice daily;
PO = oral dosing;
QD3 = 1 dose every 3 days

Tumor volumes will be measured three times a week. Tumor volume will be calculated based on two-dimensional caliper measurement with cubic millimeter volume calculated using the formula (length×width2)×0.5. Mice will be sacrificed when the tumor volumes reached 2000 mm³, which will be the survival endpoint for this study. Survival curves will be plotted using GraphPad Prism 7 software and statistical significance determined using the Holm-Sidak method, with alpha=0.05.

### Example 5: CDK4/6 Inhibitor (PF-080665) Synergizes with PD-1 Based Immune Checkpoint Blockade in the MC38 Syngeneic Mouse Tumor Model

### Overview

Palbociclib will be evaluated in the MC38 syngeneic mouse tumor model in combination with antibodies targeting PD-1, 4-1BB and OX40 to assess efficacy on primary tumor growth and survival.

### Materials and Methods

MC38 cells obtained from American Type Culture Collection (ATCC) are cultured in Roswell Park Memorial Institute (RPMI1640) supplemented with 10% fetal bovine serum (FBS). All cells are maintained in a humidified incubator at 37° C. with 5% carbon dioxide (CO₂). Female C57/BL6 mice will be obtained from Jackson Laboratories at 8 weeks of age. To generate the syngeneic model, 0.5 million MC38 tumor cells are subcutaneously implanted into the right flank of female C57/BL6 mice. Tumor bearing mice will be randomized into seven treatment groups based on average tumor sizes of approximately 70 mm³ per group, on Day 9 post tumor cell implantation. Study groups will include vehicle, 10 mg/kg palbociclib (CDK 4/6 inhibitor) twice daily by oral gavage in combination with anti-PD-1 antibody (PF-06937004) administered at 10 mg/kg IP injection every three days for three doses, 10 mg/kg palbociclib (CDK 4/6 inhibitor) twice daily by oral gavage in combination with anti-PD-1 antibody (PF-06937004) administered at 10 mg/kg IP injection and anti-4-1BB antibody (PF-07218859) administered at 3 mg/kg by IP injection every three days for three doses, 10 mg/kg palbociclib (CDK 4/6 inhibitor) twice daily by oral gavage in combination with anti-PD-1 antibody (PF-06937004) administered at 10 mg/kg IP injection and anti-OX40 antibody (PF-07201252) administered at 5 mg/kg by IP injection every three days for three doses and 10 mg/kg palbociclib (CDK 4/6 inhibitor) twice daily by oral gavage in combination with anti-PD-1 antibody (PF-06937004) administered at 10 mg/kg IP injection, anti-OX40 antibody (PF-07201252) administered at 5 mg/kg by IP injection and anti-4-1BB antibody (PF-07218859) administered at 3 mg/kg by IP injection every three days for three doses. All antibody formulations are phosphate buffered saline based while palbociclib is administered in a 0.5% methocel/Tween suspension. The treatment groups and dose regimen information are summarized in Table 14.

TABLE 14

| | | Animals/ | | |
|---|---|---|---|---|
| Group | Drug | group | Route | Regimen |
| 1 | vehicle | 10 | PO | BID |
| | | | | continuously |
| 2 | Palbociclib 10 mg/kg | 10 | PO | BID |
| | | | | continuously |
| 3 | PF-06937004 mg/kg | 10 | IP | QD3; 3 doses |
| 4 | PF-06937004 10 mg/kg + | 10 | IP + PO | QD3; 3 doses + |
| | palbociclib 10 mg/kg | | | BID |
| | | | | continuously |
| 5 | PF-06937004 10 mg/kg + | 10 | IP + IP + | QD3; 3 doses + |
| | PF-07201252 5 mg/kg + | | PO | QD3; 3 doses + |
| | palbociclib 10 mg/kg | | | BID |
| | | | | continuously |
| 6 | PF-06937004 10 mg/kg + | 10 | IP + IP + | QD3; 3 doses + |
| | PF-07218859 3 mg/kg + | | PO | QD3; 3 doses + |
| | palbociclib 10 mg/kg | | | BID |
| | | | | continuously |
| 7 | PF-06937004 10 mg/kg + | 10 | IP + IP + | QD3; 3 doses + |
| | PF-07201252 5 mg/kg + | | IP + PO | QD3; 3 doses + |
| | PF-07218859 3 mg/kg + | | | QD3; 3 doses + |
| | palbociclib 10 mg/kg | | | BID |
| | | | | continuously |

BID = twice daily;
PO = oral dosing;
QD3 = 1 dose every 3 days

Tumor volumes will be measured three times a week. Tumor volume will be calculated based on two-dimensional caliper measurement with cubic millimeter volume calculated using the formula (length×width2)×0.5. Mice will be sacrificed when the tumor volumes reached 2000 mm³, as a survival endpoint for this study. Survival curves will be plotted using GraphPad Prism 7 software and statistical significance determined using the Holm-Sidak method, with alpha =0.05.

### Example 6: CDK2/4/6 Inhibitor (PF-068736000) Synergizes with PD-1 Based Immune Checkpoint Blockade in the MC38 Syngeneic Mouse Tumor Model

### Overview

PF-06873600 will be evaluated in the MC38 syngeneic mouse tumor model in combination with antibodies targeting PD-1, 4-1BB and OX40 to assess efficacy on primary tumor growth and survival.

### Materials and Methods

MC38 cells obtained from American Type Culture Collection (ATCC) are cultured in Roswell Park Memorial Institute (RPMI1640) supplemented with 10% fetal bovine serum (FBS). All cells are maintained in a humidified incubator at 37° C. with 5% carbon dioxide (CO₂). Female C57/BL6 mice will be obtained from Jackson Laboratories at 8 weeks of age. To generate the syngeneic model, 0.5 million MC38 tumor cells are subcutaneously implanted into the right flank of female C57/BL6 mice. Tumor bearing mice will be randomized into seven treatment groups based on average tumor sizes of approximately 70 mm³ per group, on Day 9 post tumor cell implantation. Study groups will include vehicle, 30 mg/kg PF-06873600 (CDK 2/4/6 inhibitor) twice daily by oral gavage in combination with anti-PD-1 antibody (PF-06937004) administered at 10 mg/kg IP injection every three days for three doses, 30 mg/kg PF-06873600 (CDK 2/4/6 inhibitor) twice daily by oral gavage in combination with anti-PD-1 antibody (PF-06937004) administered at 10 mg/kg IP injection and anti-4-1BB antibody (PF-07218859) administered at 3 mg/kg by IP injection every three days for three doses, 30 mg/kg PF-06873600 (CDK 2/4/6 inhibitor) twice daily by oral gavage in combination with anti-PD-1 antibody (PF-06937004) administered at 10 mg/kg IP injection and anti-OX40 antibody (PF-07201252) administered at 5 mg/kg by IP injection every three days for three doses and 30 mg/kg PF-06873600 (CDK 2/4/6 inhibitor) twice daily by oral gavage in combination with anti-PD-1 antibody (PF-06937004) administered at 10 mg/kg IP injection, anti-OX40 antibody (PF-07201252) administered at 5 mg/kg by IP injection and anti-4-1BB antibody (PF-07218859) administered at 3 mg/kg by IP injection every three days for three doses. All antibody formulations are phosphate buffered saline based while PF-06873600 was administered in a 0.5% methocel/Tween suspension. The treatment groups and dose regimen information are summarized in Table 15.

TABLE 15

| | | Animals/ | | |
|---|---|---|---|---|
| Group | Drug | group | Route | Regimen |
| 1 | vehicle | 10 | PO | BID |
| | | | | continuously |
| 2 | PF-06873600 30 mg/kg | 10 | PO | BID |
| | | | | continuously |
| 3 | PF-06937004 mg/kg | 10 | IP | QD3; 3 doses |
| 4 | PF-06937004 10 mg/kg + | 10 | IP + PO | QD3; 3 doses + |
| | PF-06873600 30 mg/kg | | | BID |
| | | | | continuously |
| 5 | PF-06937004 10 mg/kg + | 10 | IP + IP + | QD3; 3 doses + |
| | PF-07201252 5 mg/kg + | | PO | QD3; 3 doses + |
| | PF-06873600 30 mg/kg | | | BID |
| | | | | continuously |
| 6 | PF-06937004 10 mg/kg + | 10 | IP + IP + | QD3; 3 doses + |
| | PF-07218859 3 mg/kg + | | PO | QD3; 3 doses + |
| | PF-06873600 30 mg/kg | | | BID |
| | | | | continuously |
| 7 | PF-06937004 10 mg/kg + | 10 | IP + IP + | QD3; 3 doses + |
| | PF-07201252 5 mg/kg + | | IP + PO | QD3; 3 doses + |
| | PF-07218859 3 mg/kg + | | | QD3; 3 doses + |
| | PF-06873600 30 mg/kg | | | BID |
| | | | | continuously |

BID = twice daily;
PO = oral dosing;
QD3 = 1 dose every 3 days

Tumor volumes will be measured three times a week. Tumor volume will be calculated based on two-dimensional caliper measurement with cubic millimeter volume calculated using the formula (length×width2)×0.5. Mice will be sacrificed when the tumor volumes reached 2000 mm³, as a survival endpoint for this study. Survival curves will be plotted using GraphPad Prism 7 software and statistical significance determined using the Holm-Sidak method, with alpha=0.05.

### Example 7: CDK2/4/6 Inhibitor (PF-068736000) Synergizes with PD-1 Based Immune Checkpoint Blockade in the 4T1 Syngeneic Mouse Tumor Model

### Overview

PF-06873600 was evaluated in the 4T1 syngeneic mouse tumor model in combination with antibodies targeting PD-L1, 4-1BB and OX40 to assess efficacy on primary tumor growth and survival. PF-06873600 in combination with immune checkpoint blockade agents led to significant tumor growth inhibition (p=0.0000001).

### Materials and Methods

4T1 cells were obtained from American Type Culture Collection (ATCC) and cultured in Roswell Park Memorial Institute (RPMI1640) supplemented with 10% fetal bovine serum (FBS). All cells were maintained in a humidified incubator at 37° C. with 5% carbon dioxide (CO2). Female C57/BL6 mice were obtained from Jackson Laboratories at 8 weeks of age. To generate the syngeneic model, 30,000 4T1 tumor cells were subcutaneously implanted into the right flank of female C57/BL6 mice. Tumor bearing mice were randomized into four treatment groups based on average tumor sizes of approximately 70 mm³ per group. Study groups included vehicle, 30 mg/kg PF-06873600 (CDK 2/4/6 inhibitor) twice daily by oral gavage, combination of 10 mg/kg anti-PD-1 antibody (PF-06937004) with 5 mg/kg anti-OX40 antibody (PF-07201252) and with 3 mg/kg anti-4-1BB antibody (PF-07218859) all administered by IP injection, and the combination of PF-06873600, anti-PD-1 antibody, anti-OX40 antibody and anti-41-BB antibody dosed as described in the cohorts above. All antibodies were administered as three doses; one every three days after the study initiation. All antibody formulations are phosphate buffered saline based while PF-06873600 was administered in a 0.5% methocel/Tween suspension. The treatment groups and dose regimen information are summarized in Table 16.

TABLE 16

| | | Animals/ | | |
|---|---|---|---|---|
| Group | Drug | group | Route | Regimen |
| 1 | vehicle | 10 | PO | BID continuously |
| 2 | PF-06873600 30 | 10 | PO | BID continuously |
| | mg/kg | | | |
| 3 | PF-06937004 | 10 | IP + IP + IP | QD3; 3 doses + |
| | 10 mg/kg + | | | QD3; 3 doses + |
| | PF-07201252 | | | QD3; 3 doses |
| | 5 mg/kg + | | | |
| | PF-07218859 | | | |
| | 3 mg/kg | | | |
| 4 | PF-06937004 | 10 | IP + IP + | QD3; 3 doses + |
| | 10 mg/kg + | | IP + PO | QD3; 3 doses + |
| | PF-07201252 | | | QD3; 3 doses + |
| | 5 mg/kg + | | | BID continuously |
| | PF-07218859 | | | |
| | 3 mg/kg + | | | |
| | PF-06873600 30 | | | |
| | mg/kg | | | |

BID = twice daily;
PO = oral dosing;
QD3 = 1 dose every 3 days

Tumor volumes were measured three times a week. Tumor volume was calculated based on two-dimensional caliper measurement with cubic millimeter volume calculated using the formula (length×width2)×0.5. Mice were sacrificed when the tumor volumes reached 2000 mm³, which was the survival endpoint for this study. Survival curves were plotted using GraphPad Prism 7 software. Statistical significance determined using the Holm-Sidak method, with alpha=0.05.

### Results

On Day 18 post-treatment initiation, tumor growth results show that treatment with the CDK2/4/6 inhibitor (PF06873600) monotherapy significantly inhibited tumor growth in the 4T1 xenograft tumor model (p=0.00000000002).

The anti-PD-1 antibody+anti-OX40 antibody+anti-4-1 BB antibody checkpoint blockade cohort resulted in significant but mild tumor growth inhibition (p=0.008). However, PF-06873600 treatment in combination with anti-PD-1 antibody +anti-OX40 antibody+anti-4-1 BB antibody showed a strong combinatorial effect with the most significant tumor growth inhibition (p=0.000000000003).

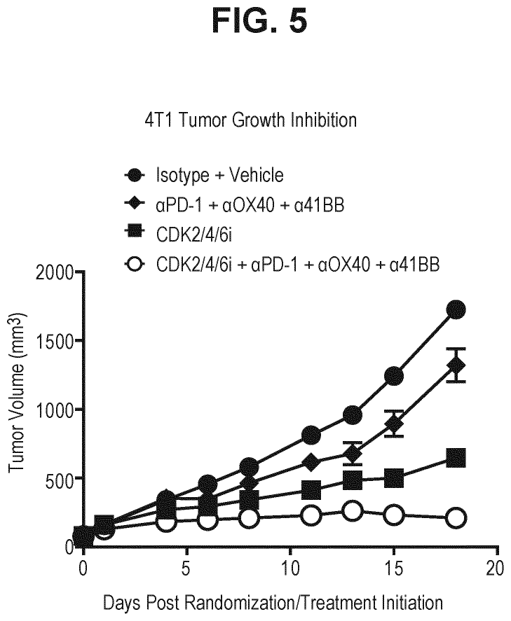

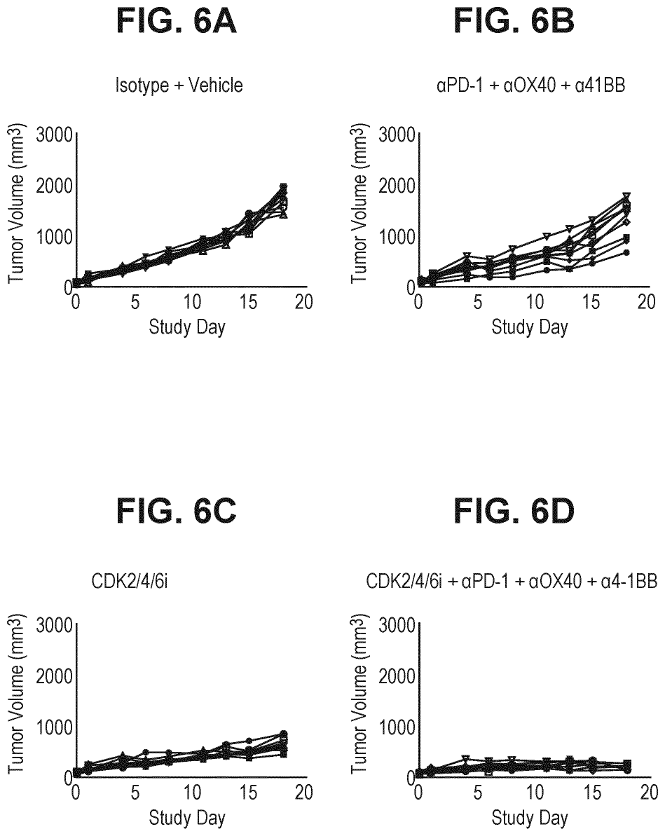

These data are summarized as mean tumor volume in FIG. 5, individual tumor volumes in FIG. 6 and absolute values in Table 17.

TABLE 17

| | | P values (vs | TGI % on |
|---|---|---|---|
| Group | Agent | vehicle) on day 18 | day 27 |
| 1 | vehicle | N/A | 0 |
| 2 | PF-06873600 30 mg/kg | 0.00000000002 | 66 |
| 3 | PF-06834635 10 mg/kg + | 0.008 | 25 |
| | PF-07201252 5 mg/kg + | | |
| | PF-07218859 3 mg/kg | | |
| 4 | PF-06834635 10 mg/kg + | 0.000000000003 | 92 |
| | PF-07201252 5 mg/kg + | | |
| | PF-07218859 3 mg/kg + | | |
| | PF-06873600 30 mg/kg | | |

All references cited herein, including patent applications, patent publications, and UniProtKB/Swiss-Prot Accession numbers cited in the specification are herein incorporated by reference in their entirety. Although the foregoing invention has been described in some detail by way of illustration and example, it will be readily apparent to those of ordinary skill in the art in light of the teachings of this invention that certain changes and modifications may be made thereto without departing from the spirit or scope of the appended claims.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. A method for treating cancer comprising administering to a subject in need thereof an amount of a cyclin dependent kinase (CDK) inhibitor in combination with an amount of a PD-1 axis binding antagonist, wherein the amounts together are effective in treating cancer, and wherein the CDK inhibitor is an inhibitor of CDK4 and CDK6 (CDK4/6 inhibitor), or an inhibitor of CDK2, CDK4 and CDK6 (CDK2/4/6 inhibitor).

2. The method of claim 1, further comprising the combined administration to the subject of an amount of:
a. an OX40 agonist;
b. a 4-1BB agonist; or
c. an OX40 agonist and a 4-1BB agonist;
wherein the amounts together are effective in treating cancer.

3. The method of claim 1, wherein the PD-1 axis binding antagonist comprises a PD-1 binding antagonist, a PD-L1 binding antagonist, or a PD-L2 binding antagonist.

4. The method of claim 3, wherein the PD-1 axis binding antagonist comprises a PD-1 binding antagonist.

5. The method of claim 4, wherein the PD-1 binding antagonist is an anti-PD-1 antibody.

6. The method of claim 5, wherein the anti-PD-1 antibody is nivolumab (MDX 1106), pembrolizumab (MK-3475), pidilizumab (CT-011), cemiplimab (REGN2810), tislelizumab (BGB-A317), spartalizumab (PDR001), RN888, mAb15, MEDI-0680 (AMP-514), BGB-108, or AGEN-2034, or a combination thereof.

7. The method of claim 3, wherein the PD-1 axis binding antagonist comprises a PD-L1 binding antagonist.

8. The method of claim 7, wherein the PD-L1 binding antagonist is an anti-PD-L1 antibody.

9. The method of claim 8, wherein the anti-PD-L1 antibody is BMS-936559 (MDX-1105), AMP-714, atezolizumab (MPDL3280A), durvalumab (MEDI4736), avelumab, or an antibody comprising a VH region produced by the expression vector with ATCC Accession No. PTA-121183 and having the VL region produced by the expression vector with ATCC Accession No. PTA-121182, or a combination thereof.

10. The method of claim 2, wherein the OX40 agonist is an anti-OX40 antibody, an OX40L agonist fragment, an OX40 oligomeric receptor, a trimeric OX40L-Fc protein or an OX40 immunoadhesin, or a combination thereof.

11. The method of claim 10, wherein the OX40 agonist is an anti-OX40 antibody.

12. The method of claim 11, wherein the anti-OX40 antibody is MEDI6469, MEDI0562, MEDI6383, MOXR0916, or GSK3174998, or a combination thereof.

13. The method of claim 2, wherein the 4-1BB agonist is an anti-4-1BB antibody.

14. The method of claim 2, wherein the 4-1BB agonist is utomilumab (PF-05082566), 1D8, 3Elor, 4B4, H4-1BB-M127, BBK2, 145501, antibody produced by cell line deposited as ATCC No. HB-11248, 5F4, C65-485, urelumab (BMS-663513), 20H4.9-IgG-1 (BMS-663031), 4E9, BMS-554271, BMS-469492, 3H3, BMS- 469497, 3E1, 53A2, or 3B8.

15. The method of claim 1, wherein the CDK inhibitor is a CDK4/6 inhibitor.

16. The method of claim 15, wherein the CDK4/6 inhibitor is palbociclib, or a pharmaceutically acceptable salt thereof.

17. The method of claim 1, wherein the CDK inhibitor is a CDK2/4/6 inhibitor.

18. The method of claim 17, wherein the CDK2/4/6 inhibitor is 6-(difluoromethyl)-8-((1R,2R)-2-hydroxy-2-methylcyclopentyl)-2-(1-(methylsulfonyl)piperidin-4-ylamino)pyrido[2,3-d]pyrimidin-7(8H)-one, or a pharmaceutically acceptable salt thereof.

19. The method of claim 1, wherein the subject is a human.

20. The method of claim 1, wherein the cancer is selected from the group consisting of brain cancer, head/neck cancer (including squamous cell carcinoma of the head and neck (SCCHN)), prostate cancer, ovarian cancer, bladder cancer (including urothelial carcinoma, also known as transitional cell carcinoma (TCC)), lung cancer (including squamous cell carcinoma, small cell lung cancer (SCLC), and non-small cell lung cancer (NSCLC)), breast cancer, bone cancer, colorectal cancer, kidney cancer, liver cancer (including hepatocellular carcinoma (HCC)), stomach cancer, pancreatic cancer, esophageal cancer, cervical cancer, sarcoma, skin cancer (including melanoma and Merkel cell carcinoma (MCC)), multiple myeloma, mesothelioma, malignant rhabdoid tumors, neuroblastoma, diffuse intrinsic pontine glioma (DIPG), carcinoma, lymphoma, diffuse large B-cell lymphoma (DLBCL), primary mediastinal B-cell lymphoma (PMBCL), follicular lymphoma, acute lymphoblastic leukemia (ALL), acute myeloid leukemia (AML), chronic lymphocytic leukemia (CLL), chronic myeloid leukemia (CML), follicular lymphoma, Hodgkin's lymphoma (HL), classical Hodgkin lymphoma (cHL), mantle cell lymphoma (MCL), multiple myeloma (MM), myeloid cell leukemia-1 protein (Mcl-1), myelodysplastic syndrome (MDS), non-Hodgkin's lymphoma (NHL), small lymphocytic lymphoma (SLL), and SWI/SNF-mutant cancer.

21-26. (canceled)
